(19) United States
(12) Patent Application Publication
(10) Pub. No.: US 2024/0390572 A1
(43) Pub. Date: Nov. 28, 2024

(54) SYSTEM OF MEDICAL INDICATORS

(71) Applicant: Star Luminal LLC, San Antonio, TX (US)

(72) Inventors: David J. Friedman, San Antonio, TX (US); Sarah Elizabeth Friedman, San Antonio, TX (US); Randall Schaefer, San Antonio, TX (US); Renae Franz, San Antonio, TX (US); Vivian Allen, San Antonio, TX (US); Cole Melton, San Antonio, TX (US); Chris Allen, San Antonio, TX (US); Asher Friedman, San Antonio, TX (US); David Underwood, San Antonio, TX (US); Laura Humphries, San Antonio, TX (US)

(21) Appl. No.: 18/671,806

(22) Filed: May 22, 2024

Related U.S. Application Data
(60) Provisional application No. 63/468,204, filed on May 22, 2023.
(60) Provisional application No. 63/570,669, filed on Mar. 27, 2024.

(51) Int. Cl. A61M 5/14; A61B 90/94

(52) U.S. Cl. CPC A61M 5/1418; A61B 90/94; A61M 2205/60

(57) ABSTRACT

A system of medical indicators includes a set of medical indicator tags, each of which includes a first plurality of identifying characteristics that are indicative of a category of therapy and a second plurality of identifying characteristics that are indicative of a specific therapy within the category of therapy, wherein the set includes a first indicator tag including the first plurality of identifying characteristics that are indicative of a first category of therapy and the second plurality of identifying characteristics that are indicative of a first specific therapy within the first category of therapy, and a second indicator tag including the first plurality of identifying characteristics that are indicative of the first category of therapy and the second plurality of identifying characteristics that are indicative of a second specific therapy within the first category of therapy.

### CROSS-REFERENCE TO RELATED APPLICATIONS

This is a non-provisional patent application relating to and claiming the benefit of commonly-owned, co-pending U.S. Provisional Patent Application Nos. 63/468,204, filed on May 22, 2023, and entitled “SYSTEM OF MEDICAL INDICATORS HAVING MULTISENSORY, MULTIPURPOSE AND MULTIFUNCTIONAL FEATURES,” and 63/570,669, filed on Mar. 27, 2024, and entitled “SYSTEM OF MEDICAL INDICATORS,” the contents of both of which are incorporated herein by reference in their entirety.

### FIELD OF THE INVENTION

The present invention relates to indicators for labelling and identifying flexible infusion therapy tubing of the type that is commonly referred to as intravenous (“IV”) tubing, and the contents thereof, using sensory stimuli, such as visual and haptic (i.e., tactile; pertaining to the sensation of touch) signatures and other identifiers. Such tubing is typically used to deliver infusion therapy by routes including, but not limited to, intravenous, intra-arterial, epidural, subcutaneous, intramuscular, intrathecal, intraperitoneal, or intramuscular routes of administration in a hospital or other setting in which infusion therapy is administered.

### BACKGROUND

Literature suggests that medical errors constitute the third highest cause of death in the United States. Among medical errors, human medication errors rank first. It has been estimated that errors occur in 53% of all administrations of therapy using infusion therapy, and that 58% of such errors occur due to errors related to line labeling. By reducing human error in the administration of infusion medication/fluid, more than 7,000 lives might be saved each year, and a similar volume of medication-related patient complications may be avoided.

What is needed is a system that reduces medical errors by (1) providing a mechanism(s) to limit medical errors during a medical crisis; (2) identifying a “safe-line” for medical personnel to use in a medical emergency; (3) creating patient, patient family or advocate awareness of therapy; (4) allowing quick, cognitive recognition of medical care at medical shift change; (5) enumerating the number of different medications/fluids in use or that have been administered; and (6) avoiding the inadvertent administration of therapy in an inappropriate type of infusion therapy line (e.g., a spinal line or an arterial line rather than a venous line).

Tubing used for infusion is generic, clear and indistinguishable from other lines in use regardless of the tubing's contents. The tubing is part of an infusion therapy line. This line is also known as an infusion set, IV set, infusion line, or simply “the line”. Infusion therapy fluids are administered through a thin, flexible transparent plastic tube. The infusion therapy tube line connects to the bag of a solution to be delivered. This line is used to infuse, continuously or intermittently, fluids or medication. It includes the following: (1) a spike and drip chamber which attaches to the bag of fluid; (2) backcheck valve which prevents fluid or medication from travelling up the line; (3) access ports, which are used to infuse secondary medications and give push medications; and (4) a roller clamp, which is used to regulate the speed of, or to stop or start, a gravity infusion. The number of lines needed for infusion therapy depends on the medical condition of the patient and how acutely ill the patient is at the time.

### SUMMARY OF THE INVENTION

The exemplary embodiments present a novel method to avoid and reduce the probability of medication errors in patients receiving infusion therapy, such as IV therapy.

The exemplary embodiments provide a mechanism for the rapid and repeated identification of the correct infusion therapy line to use in an emergency situation. In a medical crisis, care is often chaotic and multiple lines may be needed or are already in use. When an emergency medication is needed, safe, fast, and confident delivery of the medication through the correct, existing infusion therapy line(s) is essential. The correct infusion therapy line must carry a fluid which is compatible with all emergency medications and the correct infusion therapy line must be frequently recognized for repeated use, if needed.

The exemplary embodiments provide a “safe-line” or “tag” on an appropriate line that facilitates rapid identification of the most appropriate line to use in the emergency. Such a critical situation routinely arises during aeromedical and ground medical transport, but can likewise happen in a pre-hospital (e.g., ambulance or medical airlift) or hospital environment.

The exemplary embodiments further provide the application of a specific label on the line to denote the medication in use. This provides a unique ability for both the caregivers, the patient (self-care), as well as patient's advocate or family in attendance, to become informed of the medication in use. This also provides critical information regarding the initiation, duration and cessation of the medication for the patient. It confirms patient and family expectations regarding information they have been told about current and future treatment. It provides an additional check on the therapy as well as to avoid errors by caregivers.

The exemplary embodiments provide immediate cognitive awareness through a visual and haptic communication tool in situations such as at shift change or during other patient handovers, in settings such as in a pre-hospital or hospital environment. This also applies with the entry of medical personnel into the patient's room whether they are familiar with the patient, or not. This information is important since changes in patient care may have occurred in the interim since the patient was last seen or examined either by a nurse or physician (e.g., initiation of anticoagulation in a patient expected to go for surgery). Furthermore, there may have been a lack of communication regarding these changes (or information missed because of the complexity of, or deficiency in, the medical record). This immediate cognitive awareness is particularly important in an academic environment where trainee caregivers are in abundance and the risks of medical errors are higher.

The exemplary embodiments provide an identification system that embodies a mechanism for the enumeration of the number of medication(s) in use (e.g., multiple medications of the same class). In so doing this additionally allows for the awareness of either the deterioration or improvement in the patient's condition for both caregiver, patient and family. This would be the case such as when the number of vasopressor (blood pressure) tags are increased or decreased respectively. In some embodiments, the numerical ability of the blood transfusion tag allows for the tabulation of the number of units of the blood product infused as well as confirmation of the patient's blood type.

The exemplary embodiments assist in the prevention of the administration of certain medications into an inappropriate line that may result in catastrophic morbidity or death (e.g., vincristine chemotherapy given intrathecally). Just as it is important for the content of an infusion line to be easily identified by caregivers, so it is of critical importance for an incorrect line to be readily recognizable to avoid an administration error.

In an embodiment, an indicator includes a body having a first end, a second end opposite the first end, a first surface and a second surface opposite the first surface, each of which extends from the first end to the second end, and an aperture extending from the first end to the second end, the aperture being sized and shaped to receive a tube, and at least one of the first and second surfaces including a haptic signature formed thereon.

In an embodiment, the haptic signature is embossed on the at least one of the first and second surfaces. In an embodiment, the haptic signature includes a height in a range of 0.02 inch and 0.08 inch.

In an embodiment, the haptic signature is debossed on the at least one of the first and second surfaces. In an embodiment, the haptic signature includes depth in a range of 0.02 inch to 0.08 inch.

In an embodiment, the haptic signature includes a plurality of indicia elements spaced apart from one another. In an embodiment, a spacing between an adjacent pair of the plurality of indicia elements is in a range of 0.02 inch to 0.08 inch. In an embodiment, the plurality of indicia elements includes a series of elements having similar shapes, each of the series of elements includes a height that varies from the height of another of the series of elements. In an embodiment, each of the heights of the series of elements of the plurality of indicia elements is in a range of 0.005 inch to 0.08 inch.

In an embodiment, the haptic signature includes a textured portion having a grainy texture. In an embodiment, the textured portion includes a plurality of grains having sizes in a range of about 0.005 inch to about 0.010 inch.

In an embodiment, the haptic signature includes a first end, a second end opposite the first end of the haptic signature, and a width that varies along a length extending from the first and second ends of the haptic signature, wherein the width varies within a range of 0.05 inch to 0.5 inch.

In an embodiment, the haptic signature includes an embossed portion and a debossed portion, wherein the embossed portion includes a height in a range of 0.02 inch and 0.08 inch, and debossed portion includes a depth in a range of 0.02 inch to 0.08 inch.

In an embodiment, the haptic signature includes an embossed portion and a debossed portion that is debossed within the embossed portion, wherein the embossed portion includes a height in a range of 0.02 inch and 0.08 inch, and the debossed portion includes a depth in a range of 0.02 inch to 0.08 inch.

In an embodiment, the body includes a first member and a second member connected movably to the first member, wherein each of the first and second members includes an interior surface and a channel formed within the interior surface and extending from a first end thereof to a second end thereof, wherein the first and second members are movable between an open position and closed position, and wherein the channels of each of the first and second members form the aperture when the first and second members are in their closed position.

In an embodiment, the first member of the body includes at least one tab extending from the interior surface thereof, and the second member of the body includes at least one groove formed within the interior surface thereof, and wherein the at least one tab is sized and shaped to engage removably the at least one groove. In an embodiment, the body includes at least one hinge connected to the first and second members.

In an embodiment, the body includes a plurality of removable tabs.

In an embodiment, the tube is an infusion therapy tube and the haptic signature corresponds to a medication delivered by the infusion therapy tube.

In an embodiment, a combination includes at least one infusion therapy tube and a plurality of indicators, each indicator comprising a body having a first end, a second end opposite the first end, a first surface and a second surface opposite the first surface, each of which extends from the first end to the second end, and an aperture extending from the first end to the second end, the aperture being sized and shaped to receive the at least one infusion therapy tube, and at least one of the first and second surfaces including a haptic signature formed thereon, the haptic signature corresponding to a medication delivered by the at least one infusion therapy tube.

In an embodiment, the haptic signature of a first one of the plurality of indicators is different from the haptic signature of a second one of the plurality of indicators.

In an embodiment, the haptic signature of each of the plurality of indicators is different from the haptic signature of others of the plurality of indicators.

In an embodiment, the at least one infusion therapy tube includes a plurality of infusion therapy tubes, and the haptic signature of the first one of the plurality of indicators corresponds to a medication delivered by a first one of the plurality of infusion therapy tubes, and the haptic signature of the second of the plurality of indicators corresponds to another medication delivered by a second one of the plurality of infusion therapy tubes.

In an embodiment, the haptic signature of each of at least two of the plurality of indicators are identical to one another. In an embodiment, the haptic signature of each of at least three of the plurality of indicators are identical to one another.

In an embodiment, a system includes a plurality of indicator tags, each of which includes a body having a first end, a second end opposite the first end, a first surface extending from the first end to the second end, a second surface opposite the first surface and extending from the first end to the second end, and an aperture extending from the first end to the second end and intermediate the first surface and the second surface, the aperture being sized and shaped to receive a corresponding infusion therapy tube, and at least one of the first and second surfaces including a haptic signature formed thereon, wherein the plurality of indicator tags includes at least one tag of a first type and at least one tag of a second type, wherein the haptic signature of the at least one tag of the first type corresponds to a first medication, and wherein the haptic signature of the at least one tag of the second type corresponds to a second medication and is different from the haptic signature of the at least one tag of the first type.

In an embodiment, each of the plurality of indicator tags further comprises a visual indicator, wherein the visual indicator of the at least one indicator tag of the first type is different from the visual indicator of the at least one indicator tag of the second type. In an embodiment, the visual indicator includes a color, and the at least one indicator tag of the first type has a first color and the at least one indicator tag of the second type has a second color that is different from the first color. In an embodiment, the visual indicator includes a text label, and the at least one indicator tag of the first type has a first text label corresponding to the first medication, and wherein the at least one indicator tag of the second type has a second text label corresponding to the second medication.

In an embodiment, the system includes at least two indicator tags of the first type, and each of the at least two indicator tags of the first type is configured to be secured at a different location along its corresponding infusion therapy tube. In an embodiment, a first one of the indicator tags of the first type is configured to be secured to the corresponding infusion therapy tube near a connector to a bag of fluid, a second one of the indicator tags of the first type is configured to be secured to the corresponding infusion therapy tube near an infusion pump insert (if used) or a flow rate adjusting roller clamp, and a third one of the indicator tags of the first type is configured to be secured to the corresponding infusion therapy tube near an access port at a distal end of the corresponding infusion therapy tube.

In an embodiment, the haptic signature of the at least one indicator tag of the first type is an embossed haptic signature that is embossed on the at least one of the first and second surfaces of the at least one indicator tag of the first type. In an embodiment, the embossed haptic signature includes a height in a range of 0.01 inch and 0.08 inch.

In an embodiment, the haptic signature of the at least one indicator tag of the first type is a debossed haptic signature that is debossed on the at least one of the first and second surfaces of the at least one indicator tag of the first type. In an embodiment, the debossed haptic signature includes depth in a range of 0.02 inch to 0.08 inch.

In an embodiment, the haptic signature of the at least one indicator tag of the first type includes a plurality of indicia elements spaced apart from one another. In an embodiment, a spacing between an adjacent pair of the plurality of indicia elements is in a range of 0.02 inch to 0.08 inch. In an embodiment, the plurality of indicia elements includes a series of elements having similar shapes, each of the series of elements includes a height that varies from the height of another of the series of elements. In an embodiment, each of the heights of the series of elements of the plurality of indicia elements is in a range of 0.005 inch to 0.08 inch.

In an embodiment, the haptic signature of the at least one indicator tag of the first type includes a textured portion. In an embodiment, the textured portion includes a plurality of hemispherical dots having heights in a range of from 0.17 mm to 1 mm and a center-to-center spacing between adjacent dots in a range of from 1 mm to 1.5 mm. In an embodiment, the textured portion includes a plurality of hemispherical dots having heights in a range of from 1 mm to 1.2 mm and a center-to-center spacing between adjacent dots in a range of that is from 1.5 mm to 2.5 mm.

In an embodiment, the haptic signature of the at least one indicator tag of the first type includes an embossed portion and the haptic signature of the at least one indicator tag of the second type includes a textured portion.

In an embodiment, the haptic signature of the at least one indicator tag of the first type includes an embossed portion and the haptic signature of the at least one indicator tag of the second type includes a debossed portion.

In an embodiment, the haptic signature of the at least one indicator tag of the first type includes a debossed portion and the haptic signature of the at least one indicator tag of the second type includes a textured portion.

In an embodiment, a system includes at least two infusion therapy tubes, each of which has a proximal end, a distal end opposite the proximal end, and a middle portion intermediate the proximal and distal ends, wherein a first one of the infusion therapy tubes carries a first medicament, and wherein a second one of the infusion therapy tubes carries a second medicament; a plurality of indicator tags, each of which is configured to be attached to one of the at least two infusion therapy tubes, and wherein each of the indicator tags includes a haptic signature formed on at least one of the first and second surfaces and providing multi-sensory input to a user, wherein the plurality of indicator tags includes at least a first type of indicator tag and a second type of indicator tag, wherein the haptic signature and the visual indicator of the first type of indicator tag provide a first type of multi-sensory input indicative of the first medicament, wherein a first one of the first type of indicator tag is attached to the first one of the infusion therapy tubes at the proximal end of the first one of the infusion therapy tubes, wherein a second one of the first type of indicator tag is attached to the first one of the infusion therapy tubes at the middle portion of the first one of the infusion therapy tubes, wherein a third one of the first type of indicator tag is attached to the first one of the infusion therapy tubes at the distal end of the first one of the infusion therapy tubes, wherein the haptic signature and the visual indicator of the second type of indicator tag provide a second type of multi-sensory input different from the first type of multi-sensory input and indicative of the second medicament, wherein a first one of the second type of indicator tag is attached to the second one of the infusion therapy tubes at the proximal end of the second one of the infusion therapy tubes, wherein a second one of the second type of indicator tag is attached to the second one of the infusion therapy tubes at the middle portion of the second one of the infusion therapy tubes, wherein a third one of the second type of indicator tag is attached to the second one of the infusion therapy tubes at the distal end of the second one of the infusion therapy tubes.

In an embodiment, the visual indicator of each of the plurality of indicator tags includes at least one of a shape, a color, a text label, an image, or combinations thereof.

In an embodiment, a system of medical indicators, includes a set of indicator tags, wherein each of the indicator tags includes a body having a first end, a second end opposite the first end, a first surface extending from the first end to the second end, and an aperture extending from the first end to the second end, wherein the aperture is sized and shaped to receive an infusion tube, a first plurality of identifying characteristics that are indicative of a category of therapy, a second plurality of identifying characteristics that are indicative of a specific therapy within the category of therapy, wherein the set of indicator tags includes: a first indicator tag including: the first plurality of identifying characteristics that are indicative of a first category of therapy, and the second plurality of identifying characteristics that are indicative of a first specific therapy within the first category of therapy, a second indicator tag including: the first plurality of identifying characteristics that are indicative of the first category of therapy, and the second plurality of identifying characteristics that are indicative of a second specific therapy within the first category of therapy, a third indicator tag including: the first plurality of identifying characteristics that are indicative of a second category of therapy, and the second plurality of identifying characteristics that are indicative of a first specific therapy within the second category of therapy, and a fourth indicator tag including: the first plurality of identifying characteristics that are indicative of the second category of therapy, and the second plurality of identifying characteristics that are indicative of a second specific therapy within the second category of therapy.

In some embodiments, the first plurality of identifying characteristics includes an indicator tag shape and an indicator tag color.

In some embodiments, the second plurality of identifying characteristics includes an icon and a text label. In some embodiments, the icon is a haptic icon. In some embodiments, each of the indicator tags further comprises a second surface opposite the first surface, wherein the icon and the text label are positioned on the first surface, and wherein each of the indicator tags further includes a writable portion on the second surface.

In some embodiments, the first category of therapy is a first one of high-alert therapy, non-high-alert therapy, or specialty therapy, and the second category of therapy is a second one of high-alert therapy, non-high-alert therapy, or specialty therapy. In some embodiments, the first category of therapy is high-alert therapy, and the first plurality of identifying characteristics that are indicative of the first category of therapy comprise a triangular tag shape and a red tag color. In some embodiments, one of: (a) the second category of therapy is non-high-alert therapy and the first plurality of identifying characteristics that are indicative of the second category of therapy includes a circular tag shape and an orange tag color, or (b) the second category of therapy is specialty therapy and the first plurality of identifying characteristics that are indicative of the second category of therapy includes a square tag shape and a blue tag color. In some embodiments, the first specific therapy within the first category of therapy is one of heparin, insulin, an opioid, oxytocin, total parenteral nutrition, magnesium sulfate, a sedative, or chemotherapy.

In some embodiments, the system also includes a fifth indicator tag comprising identifying characteristics that are indicative of an emergency line. In some embodiments, a system of medical indicators includes a set of indicator tags, wherein each of the indicator tags includes a body having a first end, a second end opposite the first end, a first surface extending from the first end to the second end, and an aperture extending from the first end to the second end, wherein the aperture is sized and shaped to receive an infusion tube, wherein the aperture is configured such that each of the indicator tags can be secured to at least a first size of the infusion tube and a second size of the infusion tube without sliding along the infusion tube or restricting fluid flow within the infusion tube, wherein the first size of the infusion tube has a first outside diameter, wherein the second size of the infusion tube has a second outside diameter, and wherein the second outside diameter is greater than the first outside diameter, and a plurality of identifying characteristics that are indicative of a therapy.

In some embodiments, the first size of the infusion tube is a small adult-size infusion tube, and the second size of the infusion tube is a large adult-size infusion tube.

In some embodiments, the aperture has a generally rectangular cross-section including shorter sides and longer sides. In some embodiments, the aperture has a rectangular cross-section with rounded corners. In some embodiments, the shorter sides have a shorter side length that is about equal to the first outside diameter. In some embodiments, the shorter side length is in a range of from 0.9 times the first outside diameter to 1 times the first outside diameter. In some embodiments, the longer sides have a longer side length that is in a range of from 1.05 times the second outside diameter to 1.3 times the second outside diameter. In some embodiments, the shorter sides have a shorter side length that is in a range of from 0.9 times the first outside diameter to 1 times the first outside diameter. In some embodiments, the first outside diameter is 0.145 inches and the second outside diameter is 0.165 inches. In some embodiments, the shorter sides have a shorter side length that is in a range of from 0.14 inches to 0.15 inches, and the longer sides have a longer side length that is in a range of from 0.175 inches to 0.185 inches.

### BRIEF DESCRIPTION OF THE DRAWINGS

The present invention will be further explained with reference to the attached drawings, wherein like structures are referred to by like numerals throughout the several views. The drawings shown are not necessarily to scale, with emphasis instead generally being placed upon illustrating the principles of the present invention.

FIG. 1 is a top perspective view of an embodiment of an infusion therapy tubing indicator;

FIG. 2 is top plan view thereof, the bottom plan view being a mirror image thereto;

FIG. 3 is a front elevation view thereof, the rear elevational view being a mirror image thereto;

FIG. 4 is a left side elevational view thereof, the right side elevational view being a mirror image thereof;

FIG. 5 is a top perspective view thereof, with the indicator being attached to tubing;

FIG. 6 is a top perspective view thereof, with the indicator including a haptic signature on a surface thereof;

FIG. 7 is a top perspective view of another embodiment of an infusion therapy tubing indicator in a closed position;

FIG. 8 is a top plan view thereof;

FIG. 9 is a bottom plan view thereof;

FIG. 10 is a front elevational view thereof;

FIG. 11 is a rear elevational view thereof;

FIG. 12 is a left side elevational view thereof;

FIG. 13 is a right side elevational view thereof;

FIG. 14 is a top perspective view thereof, with the indicator being attached to tubing;

FIG. 15 is a top perspective view thereof, with the indicator including a haptic signature on a surface thereof;

FIG. 16 is a top perspective view of the infusion therapy tubing indicator of FIG. 7 but in an open position;

FIG. 17 is a top plan view thereof;

FIG. 18 is a bottom plan view thereof;

FIG. 19 is a front elevational view thereof;

FIG. 20 is a rear elevational view thereof;

FIG. 21 is a left side elevational view thereof;

FIG. 22 is a right side elevational view thereof;

FIG. 23 is a bottom plan view of another embodiment of the infusion therapy tubing indicator, with interior fastening elements;

FIG. 24A is a front perspective view of another embodiment of an infusion therapy tubing indicator including an exemplary haptic signature/visual for an antihypertensive;

FIG. 24B is a rear perspective view of the infusion therapy tubing indicator of FIG. 24A;

FIG. 25A is a front perspective view of another embodiment of an infusion therapy tubing indicator including an exemplary haptic signature/visual for an antibiotic;

FIG. 25B is a rear perspective view of the infusion therapy tubing indicator of FIG. 25A;

FIG. 26A is a front perspective view of another embodiment of a tubing indicator including an exemplary haptic signature/visual for an arterial line;

FIG. 26B is a rear perspective view of the tubing indicator of FIG. 26A;

FIG. 27A is a front perspective view of another embodiment of an infusion therapy tubing indicator including an exemplary haptic signature/visual for a venous line;

FIG. 27B is a rear perspective view of the infusion therapy tubing indicator of FIG. 27A;

FIG. 28A is a front perspective view of another embodiment of a tubing indicator including an exemplary haptic signature/visual for spinal administration;

FIG. 28B is a rear perspective view of the tubing indicator of FIG. 28A;

FIG. 29 is a front perspective view of another embodiment of a tubing indicator including an exemplary haptic signature/visual for a port cover (Do Not Use);

FIG. 30 is a front perspective view of another embodiment of a tubing indicator including an exemplary haptic signature/visual for incompatible medications (i.e., to be administered alone);

FIG. 31 is a front perspective view of another embodiment of an infusion therapy tubing indicator including an exemplary haptic signature/visual for an antiarrhythmic;

FIG. 32 is a front perspective view of another embodiment of an infusion therapy tubing indicator including an exemplary haptic signature/visual for an anticoagulant;

FIG. 33 is a front perspective view of another embodiment of an infusion therapy tubing indicator including an exemplary haptic signature/visual for an opioid (e.g., a narcotic);

FIG. 34 is a front perspective view of another embodiment of an infusion therapy tubing indicator including an exemplary haptic signature/visual for blood transfusion;

FIG. 35 is a front perspective view of another embodiment of an infusion therapy tubing indicator including an exemplary haptic signature/visual for a vasopressor;

FIG. 36 is a front perspective view of another embodiment of an infusion therapy tubing indicator including an exemplary haptic signature/visual for a paralytic;

FIG. 37 is a front perspective view of another embodiment of an infusion therapy tubing indicator including an exemplary haptic signature/visual for insulin;

FIG. 38 is a front perspective view of another embodiment of an infusion therapy tubing indicator including an exemplary haptic signature/visual for a sedative (i.e., sedation);

FIG. 39 is a front perspective view of another embodiment of an infusion therapy tubing indicator including an exemplary haptic signature/visual for chemotherapy;

FIG. 40 is a front perspective view of another embodiment of an infusion therapy tubing indicator including an exemplary haptic signature/visual for total parenteral nutrition (TPN);

FIG. 41 is a front perspective view of another embodiment of an infusion therapy tubing indicator including an exemplary haptic signature/visual for pediatric dosing;

FIG. 42 is a front perspective view of another embodiment of an infusion therapy tubing indicator including an exemplary haptic signature/visual for potassium;

FIG. 43 is a front perspective view of another embodiment of an infusion therapy tubing indicator including an exemplary haptic signature/visual for obstetric administration;

FIG. 44 is a photograph of a prototype example of an exemplary infusion therapy tubing indicator;

FIG. 45 is a perspective view of another embodiment of infusion therapy tubing indicators;

FIG. 46 is a perspective view of another embodiment of infusion therapy tubing indicators;

FIG. 47A is a perspective view of an embodiment of a high-alert infusion therapy tubing indicator;

FIG. 47B is a perspective view of an embodiment of a non-high-alert infusion therapy tubing indicator;

FIG. 47C is a perspective view of an embodiment of a specialty infusion therapy tubing indicator;

FIG. 47D is a perspective view of an embodiment of an oral medication indicator tag;

FIG. 48A is a perspective view of an embodiment of a high-alert infusion therapy tubing indicator;

FIG. 48B is a perspective view of an embodiment of a non-high-alert infusion therapy tubing indicator;

FIG. 48C is a perspective view of an embodiment of a specialty infusion therapy tubing indicator;

FIG. 48D is a perspective view of an embodiment of an oral medication indicator tag;

FIG. 49A is a front perspective view of an embodiment of a high-alert infusion therapy tubing indicator;

FIG. 49B is a rear perspective view of the high-alert infusion therapy tubing indicator shown in FIG. 49A;

FIG. 49C is a front perspective view of the high-alert infusion therapy tubing indicator shown in FIG. 49A, the indicator being positioned in an open position;

FIG. 50A is a front view of an embodiment of an infusion therapy tubing indicator including exemplary indicia for heparin;

FIG. 50B is a front view of an embodiment of an infusion therapy tubing indicator including exemplary indicia for insulin;

FIG. 50C is a front view of an embodiment of an infusion therapy tubing indicator including exemplary indicia for oxytocin;

FIG. 50D is a front view of an embodiment of an infusion therapy tubing indicator including exemplary indicia for total parenteral nutrition;

FIG. 50E is a front view of an embodiment of an infusion therapy tubing indicator including exemplary indicia for magnesium sulfate;

FIG. 50F is a front view of an embodiment of an infusion therapy tubing indicator including exemplary indicia for an opioid;

FIG. 50G is a front view of an embodiment of an infusion therapy tubing indicator including exemplary indicia for a sedative;

FIG. 50H is a front view of an embodiment of an infusion therapy tubing indicator including exemplary indicia for a vasopressor;

FIG. 50I is a front view of an embodiment of an infusion therapy tubing indicator including exemplary indicia for chemotherapy;

FIG. 51A is a front view of an embodiment of an infusion therapy tubing indicator including exemplary indicia for a transfusion;

FIG. 51B is a front view of an embodiment of an infusion therapy tubing indicator including exemplary indicia for lipids;

FIG. 52A is a front view of an embodiment of an infusion therapy tubing indicator including exemplary indicia for an antibiotic;

FIG. 52B is a front view of an embodiment of an infusion therapy tubing indicator including exemplary indicia for a biologic;

FIG. 53A is a front view of an embodiment of an infusion therapy tubing indicator including exemplary indicia for a line that is to be used for emergency medication;

FIG. 53B is a front view of an embodiment of an infusion therapy tubing indicator including exemplary indicia for a line that is not to be used;

FIG. 53C is a rear view of an embodiment of an infusion therapy tubing indicator including exemplary indicia for a line that is not to be used;

FIG. 54A is a front view of an embodiment of a set of infusion therapy indicators;

FIG. 54B is a rear view of an embodiment of a set of infusion therapy indicators;

FIG. 54C is a front view of an embodiment of an infusion therapy tubing indicator including exemplary indicia for an antibiotic;

FIG. 54D is a rear view of the infusion therapy tubing indicator shown in FIG. 54C;

FIG. 54E is a front view of an embodiment of an infusion therapy tubing indicator including exemplary indicia for a transfusion;

FIG. 54F is a rear view of the infusion therapy tubing indicator shown in FIG. 54E;

FIG. 54G is a front view of an embodiment of an infusion therapy tubing indicator including exemplary indicia for chemotherapy;

FIG. 54H is a rear view of the infusion therapy tubing indicator shown in FIG. 54G;

FIG. 54I is a front view of an embodiment of an infusion therapy tubing indicator including exemplary indicia for heparin;

FIG. 54J is a rear view of the infusion therapy tubing indicator shown in FIG. 54I;

FIG. 54K is a front view of an embodiment of an infusion therapy tubing indicator including exemplary indicia for an emergency administration line;

FIG. 54L is a rear view of the infusion therapy tubing indicator shown in FIG. 54K;

FIG. 54M is a front view of an embodiment of an infusion therapy tubing indicatir including exemplary indicia for a biologic;

FIG. 55A is a section view of an exemplary infusion therapy tubing indicator as secured to an infusion therapy tube of a first size;

FIG. 55B is a section view of the exemplary infusion therapy tubing indicator shown in FIG. 55A as secured to an infusion therapy tube of a second size that is larger than the first size as shown in FIG. 55A;

FIG. 56A is a first portion of a table showing several examples of haptic signatures, along with information regarding their specific applications, metaphors, haptic and visual signatures;

FIG. 56B is a second portion of the table beginning in FIG. 56A;

FIG. 56C is a third portion of the table beginning in FIG. 56A;

FIG. 56D is a fourth portion of the table beginning in FIG. 56A;

FIG. 56E is a fifth portion of the table beginning in FIG. 56A.

FIG. 57A is a representative image of a clinician working with infusion therapy lines that have been labeled with tape; and

FIG. 57B is a representative image of a clinician working with infusion therapy lines that have been labeled with indicators in accordance with exemplary embodiments described herein.

### DETAILED DESCRIPTION OF THE DRAWINGS

Multiple infusion therapy tubing indicators (sometimes referred to herein as “tags”) are disclosed. Such indicators are affixed to infusion therapy tubing (which is commonly referred to as “intravenous tubing” or “IV tubing”) in a hospital or other location where infusion therapy is administered. Such tubing is commonly used for delivery of infusion therapy via, but is not limited to, intravenous, intraarterial, epidural, subcutaneous, intramuscular, intrathecal, or intramuscular routes of administration. The exemplary indicators function to provide to caregivers and patients more efficient, reliable and accurate identification of medications or other substances being conveyed to the patient via the infusion therapy tubing.

In some embodiments, the infusion therapy tubing indicators include haptic (i.e., tactile; pertaining to the sensation of touch) features. This assists with recognition of the indicator and, thereby, the medication, by feel, as well as in conditions of low light. In some embodiments, the indicators are fabricated to convey multi-sensory communications to a user (e.g., a physician, nurse, or other medical clinician treating a patient) to provide further recognition regarding the medication or other substances contained in the infusion therapy tubing. In some embodiments, the multi-sensory communication may include symbols, print/text, color, shape, luminosity (e.g., generated by a luminescent) and/or touch/tactile stimuli. Specific combinations of multi-sensory communications correspond and alert the caregivers to different classes or types of medication or fluids. This reminds or warns the user of the contents of the infusion therapy tubing, in order to take appropriate action in connection therewith, should this be necessary (e.g., the insulin tag has a grainy feel/texture resembling sugar). Use of the infusion therapy tubing indicators thereby minimizes or prevents medication-related errors that can be catastrophic, and even fatal, to the patient.

In some embodiments, the indicators include a system of infusion therapy line tags with haptic signatures for the use of differentiating medications or fluids through various sensory modalities including visual symbols, luminosity, print/text, color, textures, luminosity and touch. In some embodiments, each distinct haptic signature is located on its own tag that is configured to be placed on one or more locations of the infusion therapy tubing to decrease the likelihood of line mix-ups and errors, particularly in complex clinical situations when multiple infusion therapy lines are in use.

In some embodiments, the indicators are plastic tags that are configured to be secured to an infusion therapy line. In some embodiments, the plastic tags are injection molded. In some embodiments, the tags are clip-on tags (that cannot be removed). In some embodiments, one or more separate clips is/are used to attach the tags to the infusion therapy line. In some embodiments, the clips are removable. In some embodiments, a face of the tag includes a centralized haptic signature that fits the thumb of a user. In some embodiments, an outer ring surrounding the haptic signature includes a label at the top and pull/twist off indicators on each side that numerically indicate the number of similar lines (or class of drug) in use. In some embodiments, a back of the tag includes two snap-on features that allow for easy connection to infusion therapy tubing. In some embodiments, the haptic tags are sized so as to fit the fingers of most males and most females. In some embodiments, the haptic tags are sized so as to fit different finger sizes within a range spanning, at the smallest end, the 5th percentile of female fingers and, at the largest end, the 95th percentile of male fingers. In one embodiment, the haptic tags are approximately 2 inches tall by 1.5 inches wide, which is a size that accounts for the 5th percentile female fingers and the 95th percentile male fingers.

The indicators (e.g. tags) allow medical personnel to easily locate and identify infusion therapy lines through their corresponding haptic signatures and visual cues. Tags closer to the patient allow for quick and easy identification. A user may identify a specific tag through a range of sensory modalities including color, label, luminosity, symbol, or texture. The haptic signature is positioned so that a user's thumb can easily “read” with natural thumb flexion while still being able to see the label and color associated with the tag. In some embodiments, a haptic signature that is sized and shaped to fit a user's thumb provides a semantic cue to induce the user to place the user's thumb over the haptic signature.

In various embodiments, an individual tag includes a color, text, and a specific haptic signature to identify the drug with which it is associated. Haptic signatures include textures, icons, and metaphors that are meaningful and familiar to clinical professionals (e.g. grainy feel/texture resembling sugar for insulin line; prickly feel/texture for narcotic lines; wavy feel/texture for anticoagulants) To differentiate shapes and prevent confusion between tags, haptic signatures may include varying heights, spacing, textures, degrees of roughness, and/or sizes in various embodiments.

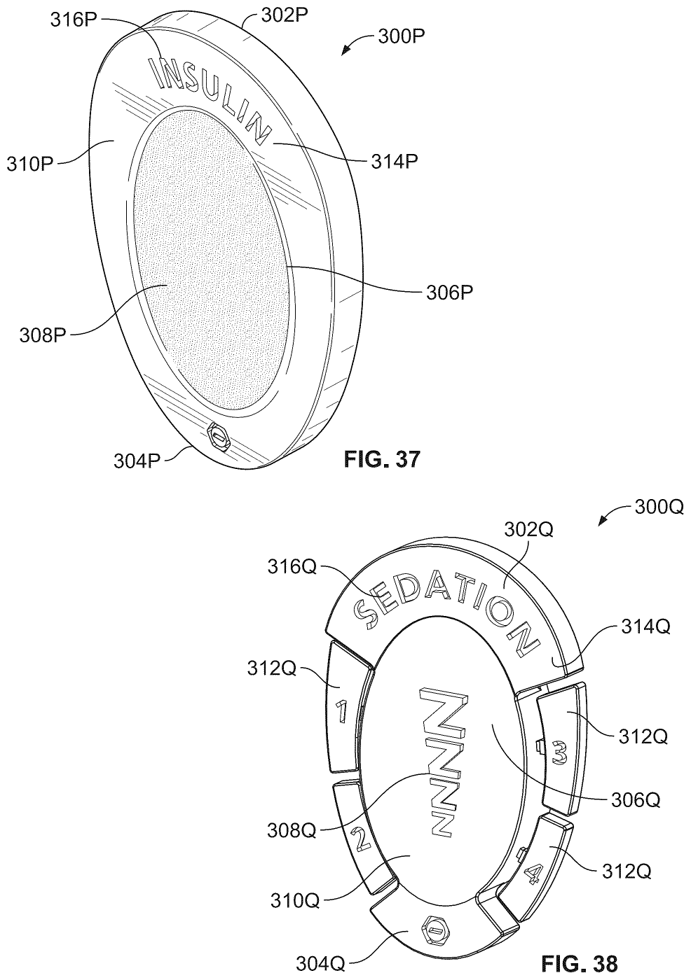

In some embodiments, each tag (except for insulin) includes sequentially numbered pull/twist-off tabs to indicate the number of infusion therapy bags a patient has used in a day. In some embodiments, an insulin tag does not include any pull/twist-off tabs to prevent confusion and overdosing (see FIG. 37). In some embodiments, blood transfusion lines have different tabs (see FIG. 34) that allow medical personnel to indicate the blood type of the patient as well as how many transfusions they have received in a day (e.g., by removing one tab at the time of each transfusion, by removing a quantity of tabs corresponding to the number of transfusions that have been received, etc.).

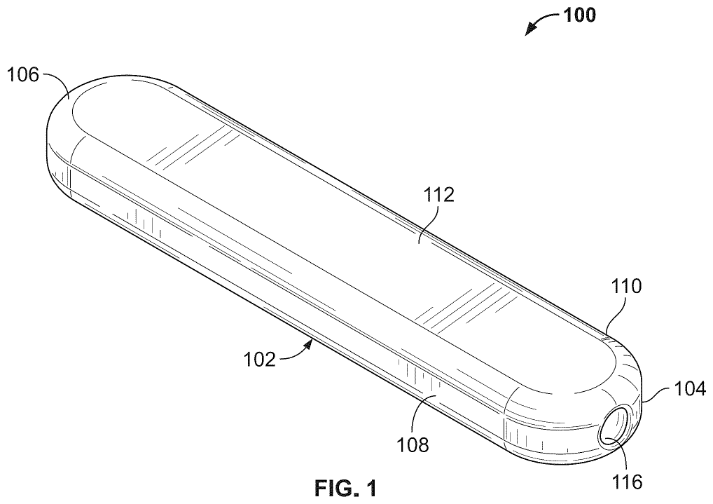

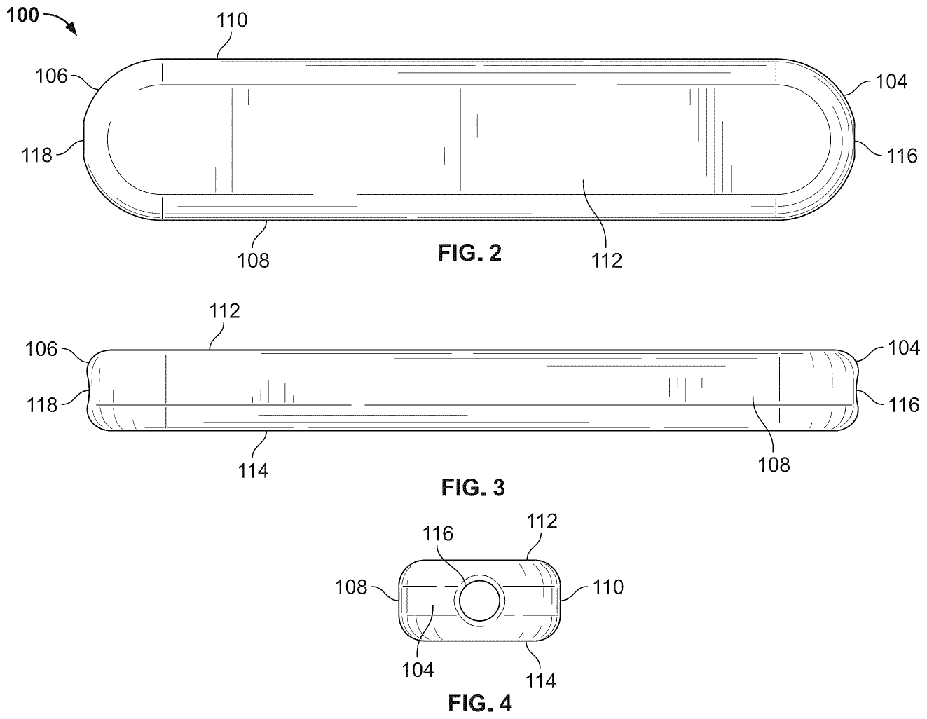

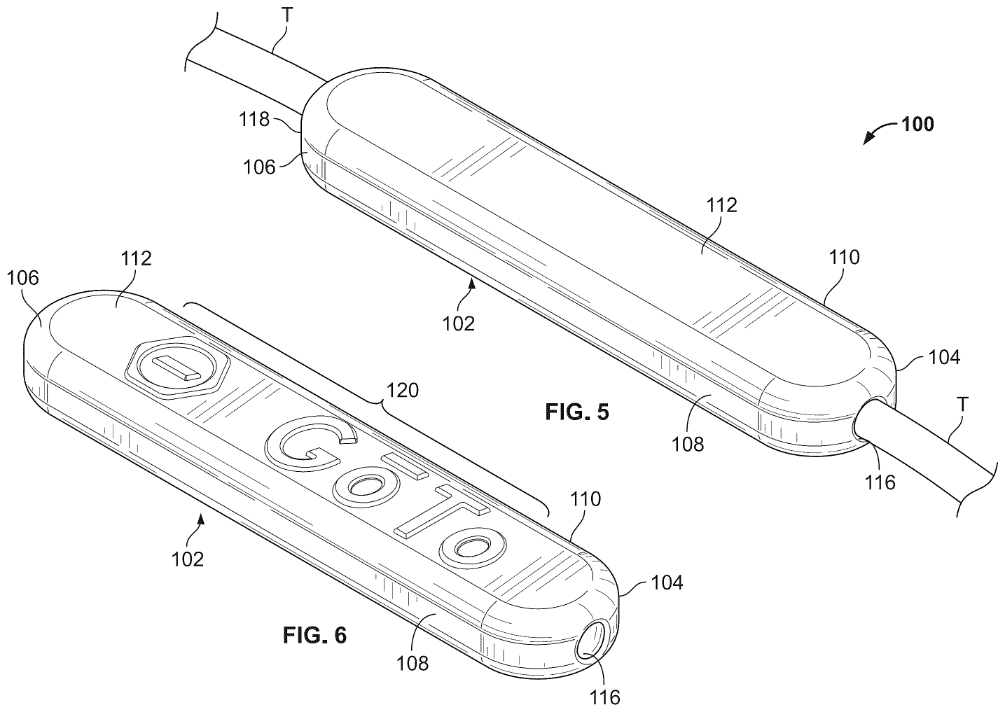

FIGS. 1-6 illustrate a first exemplary embodiment of an infusion therapy tubing indicator 100 according to the present disclosure. The indicator 100 includes a body 102 that is monolithically formed, and has two opposed ends 104, 106. Opposed sides 108, 110 extend between ends 104, 106. Opposed top and bottom surfaces 112, 114 extend between ends 104, 106 and are bordered by sides 108, 110. A central, longitudinal aperture extends through the body 102 from the end 104 to the end 106, such that the end 104 defines an aperture 116 therein, and the end 106 defines an aperture 118 therein. Apertures 116, 118 are dimensioned so as to receive a length of infusion therapy tubing T therethrough. More particularly, the infusion therapy tubing T is inserted through aperture 116, extended along the interior of the indicator body 102, and exits therefrom through aperture 118, as shown in FIG. 5.

At least one of the top or bottom surfaces 112, 114 on the indicator body 102 includes a haptic signature 120. The haptic signature 120 may include images, designs, numbers, letters, punctuation marks, patterns, other indicia, or any combination thereof (collectively “indicia”). The haptic signature 120 is formulated to convey to a user (e.g., a physician, nurse or other medical personnel treating a patient) the nature of the medication or other substance that is contained within the infusion therapy tubing T and being delivered to the patient thereby. The haptic signature 120 is formed on the surface(s) 112, 114 of the indicator body 102 by any means known in the art, such as, for example, decorating, embossing, 3D printing, engraving, or as part of the injection-molding process.

In some embodiments, at least one indicator 100 within a set of indicators includes a haptic signature 120 that is embossed on (i.e., raised from) the corresponding one of the surfaces 112, 114 of the indicator body 102. In some embodiments, such a haptic signature 120 is embossed to a height that is selected to enable a user to identify indicia in the haptic signature 120, and thereby to identify the medicament or other substance that is contained within the tubing T to which the indicator 100 is attached. In some embodiments, the embossing height is in a range of between 0.02 inch and 0.08 inch. In some embodiments, the embossing height is in a range of between 0.02 inch and 0.04 inch. In some embodiments, the embossing height is in a range of between 0.04 inch and 0.06 inch. In some embodiments, the embossing height is in a range of between 0.06 inch and 0.08 inch. In some embodiments, the embossing height is in a range of between 0.02 inch and 0.06 inch. In some embodiments, the embossing height is in a range of between 0.04 inch and 0.08 inch. In some embodiments, the embossing height is in a range of between 0.01 inch and 0.07 inch. In some embodiments, the embossing height is in a range of between 0.01 inch and 0.03 inch. In some embodiments, the embossing height is in a range of between 0.01 inch and 0.04 inch. In some embodiments, the embossing height is in a range of between 0.01 inch and 0.05 inch. In some embodiments, the embossing height is in a range of between 0.01 inch and 0.06 inch. In some embodiments, the embossing height is in a range of between 0.05 inch and 0.07 inch. In some embodiments, the embossing height is in a range of between 0.01 inch and 0.08 inch. In some embodiments, the embossing height is in a range of between 0.03 inch and 0.07 inch. In some embodiments, the embossing height is about 0.01 inch. In some embodiments, the embossing height is about 0.015 inch. In some embodiments, the embossing height is about 0.02 inch. In some embodiments, the embossing height is about 0.03 inch. In some embodiments, the embossing height is about 0.04 inch. In some embodiments, the embossing height is about 0.05 inch. In some embodiments, the embossing height is about 0.06 inch. In some embodiments, the embossing height is about 0.07 inch. In some embodiments, the embossing height is about 0.08 inch. In some embodiments, the embossing height is in a range of between 0.5 and 0.55 mm. In some embodiments, the embossing height is about 0.5 mm. In some embodiments, the embossing height is 0.5 mm. In some embodiments, the embossing height is 0.51 mm. In some embodiments, the embossing height is in a range of between 0.9 mm and 1.1 mm. In some embodiments, the embossing height is about 1 mm. In some embodiments, the embossing height is 1 mm. In some embodiments, the embossing height is in a range of between 1.4 and 1.6 mm. In some embodiments, the embossing height is about 1.5 mm. In some embodiments, the embossing height is 1.5 mm. In some embodiments, the embossing height is in a range of between 1.15 mm and 1.35 mm. In some embodiments, the embossing height is about 1.25 mm. In some embodiments, the embossing height is 1.25 mm. In some embodiments, the embossing height is in a range of between 0.3 mm and 0.4 mm. In some embodiments, the embossing height is in a range of between 0.3 mm and 0.36 mm. In some embodiments, the embossing height is about 0.33 mm. In some embodiments, the embossing height is 0.33 mm. In some embodiments, the embossing height is in a range of between 1.4 mm and 1.5 mm. In some embodiments, the embossing height is in a range of between 1.4 mm and 1.44 mm. In some embodiments, the embossing height is about 1.42 mm. In some embodiments, the embossing height is 1.42 mm.

In some embodiments, at least one indicator 100 within a set of indicators includes a haptic signature 120 that is debossed on (i.e., depressed from) the corresponding one of the surfaces 112, 114 of the indicator body 102. In some embodiments, such a haptic signature 120 is debossed to a depth that is selected so as to enable a user to identify indicia in the haptic signature 120, and thereby to identify the medicament or other substance that is contained within the tubing T to which the indicator 100 is attached. In some embodiments, the debossing depth is in a range of between 0.02 inch and 0.08 inch. In some embodiments, the debossing depth is in a range of between 0.02 inch and 0.04 inch. In some embodiments, the debossing depth is in a range of between 0.04 inch and 0.06 inch. In some embodiments, the debossing depth is in a range of between 0.06 inch and 0.08 inch. In some embodiments, the debossing depth is in a range of between 0.02 inch and 0.06 inch. In some embodiments, the debossing depth is in a range of between 0.04 inch and 0.08 inch. In some embodiments, the debossing depth is in a range of between 0.4 mm and 0.6 mm. In some embodiments, the debossing depth is in a range of between 0.45 mm and 0.55 mm. In some embodiments, the debossing depth is about 0.5 mm. In some embodiments, the debossing depth is 0.5 mm. In some embodiments, the debossing depth is a range of between 0.8 mm and 1.2 mm. In some embodiments, the debossing depth is in a range of between 0.9 mm and 1.1 mm. In some embodiments, the debossing depth is about 1 mm. In some embodiments, the debossing depth is 1 mm.

In some embodiments, at least one indicator 100 within a set of indicators includes a haptic signature 120 that includes a grainy texture formed on (i.e., raised from) the corresponding one of the surfaces 112, 114 of the indicator body 102. In some embodiments, such a haptic signature 120 includes a grainy texture that is selected so as to enable a user to identify indicia in the haptic signature 120, and thereby to identify the medicament or other substance that is contained within the tubing T to which the indicator 100 is attached. In some embodiments, the grainy texture includes individual grains of varying sizes. In some embodiments, the grainy texture has a texture comparable to that of 100 grit sandpaper. In some embodiments, the grainy texture includes grains having sizes that are in a range of between 0.005 inch and 0.010 inch. In some embodiments, the grainy texture includes grains having sizes that are in a range of between 0.005 inch and 0.006 inch. In some embodiments, the grainy texture includes grains having sizes that are in a range of between 0.006 inch and 0.007 inch. In some embodiments, the grainy texture includes grains having sizes that are in a range of between 0.007 inch and 0.008 inch. In some embodiments, the grainy texture includes grains having sizes that are in a range of between 0.008 inch and 0.009 inch. In some embodiments, the grainy texture includes grains having sizes that are in a range of between 0.009 inch and 0.010 inch. In some embodiments, the grainy texture includes grains having sizes that are in a range of between 0.005 inch and 0.007 inch. In some embodiments, the grainy texture includes grains having sizes that are in a range of between 0.006 inch and 0.008 inch. In some embodiments, the grainy texture includes grains having sizes that are in a range of between 0.007 inch and 0.009 inch. In some embodiments, the grainy texture includes grains having sizes that are in a range of between 0.008 inch and 0.010 inch. In some embodiments, the grainy texture includes grains having sizes that are in a range of between 0.005 inch and 0.008 inch. In some embodiments, the grainy texture includes grains having sizes that are in a range of between 0.006 inch and 0.009 inch. In some embodiments, the grainy texture includes grains having sizes that are in a range of between 0.007 inch and 0.010 inch. In some embodiments, the grainy texture includes grains having sizes that are in a range of between 0.005 inch and 0.009 inch. In some embodiments, the grainy texture includes grains having sizes that are in a range of between 0.006 inch and 0.010 inch.

In some embodiments, the grainy texture includes a plurality of generally hemispherical “dots” that are embossed on (i.e., raised from) a corresponding one of the surfaces 112, 114 of the indicator body 102. In some embodiments, each such dot has a diameter and a height from the corresponding one of the surfaces 112, 114 that is half of the diameter. In some embodiments, such a plurality of embossed dots forms a matrix such that each such dot produces a pronounced point load at a corresponding location on the pad of a fingertip that is contacting the matrix of dots, thereby enhancing tactile sensation. In some embodiments, the diameter of each dot is from 0.34 mm to 3 mm, or from 1 mm to 3 mm, or from 1.66 mm to 3 mm, or from 2.33 mm to 3 mm, or from 0.34 mm to 2.33 mm, or from 1 mm to 2.33 mm, or from 1.66 mm to 2.33 mm, or from 0.34 mm to 1.66 mm, or from 0.34 mm to 1 mm, or from 1 mm to 1.66 mm. In some embodiments, a matrix including dots of heights in a range of from 0.17 mm to 1 mm (i.e., having diameters in a range of from 0.34 mm to 2 mm) has a center-to-center spacing between adjacent dots that is from 1 mm to 1.5 mm, or from 1 mm to 1.25 mm, or from 1.25 mm to 1.5 mm. In some embodiments, a matrix including dots of heights in a range of from 1 mm to 1.2 mm (i.e., having diameters in a range of from 2 mm to 3 mm) has a center-to-center spacing between adjacent dots that is from 1.5 mm to 2.5 mm, or from 1.5 mm to 2 mm, or from 2 mm to 2.5 mm.

In some embodiments, at least one indicator 100 within a set of indicators includes a haptic signature 120 that includes a plurality of similar individual indicia elements (e.g., lines, curves, letters, symbols, etc.) that are spaced apart from one another along the corresponding one of the surfaces 112, 114 of the indicator body 102. In some embodiments, each such indicia element is embossed from the corresponding one of the surfaces 112, 114 to a height such as one of the embossing heights discussed above. In some embodiments, each such indicia element is debossed from the corresponding one of the surfaces 112, 114 to a depth such as one of the debossing depths discussed above. In some embodiments, such a haptic signature 120 includes indicia elements that are spaced apart from one another by a spacing distance that is selected so as to enable a user to identify the indicia elements in the haptic signature 120 as similar indicia elements that are spaced apart from one another, and thereby to identify the medicament or other substance that is contained within the tubing T to which the indicator 100 is attached. In some embodiments, the spacing distance is in a range of between 0.02 inch and 0.08 inch. In some embodiments, the spacing distance is in a range of between 0.02 inch and 0.04 inch. In some embodiments, the spacing distance is in a range of between 0.04 inch and 0.06 inch. In some embodiments, the spacing distance is in a range of between 0.06 inch and 0.08 inch. In some embodiments, the spacing distance is in a range of between 0.02 inch and 0.06 inch. In some embodiments, the spacing distance is in a range of between 0.04 inch and 0.08 inch. In some embodiments, the spacing distance is in a range of between 0.06 inch and 0.12 inch. In some embodiments, the spacing distance is in a range of between 0.06 inch and 0.08 inch. In some embodiments, the spacing distance is in a range of between 0.08 inch and 0.10 inch. In some embodiments, the spacing distance is in a range of between 0.10 inch and 0.12 inch. In some embodiments, the spacing distance is in a range of between 0.06 inch and 0.10 inch. In some embodiments, the spacing distance is in a range of between 0.08 inch and 0.12 inch.

In some embodiments, at least one indicator 100 within a set of indicators includes a haptic signature 120 that includes a series of similar individual indicia elements (e.g., lines, curves, letters, symbols, etc.) that are spaced apart from one another along the corresponding one of the surfaces 112, 114 of the indicator body 102 and are embossed from the corresponding one of the surfaces 112, 114 to an embossing height that varies among the indicia elements (e.g., each of the indicia elements has a height that is different from the heights of some or all of the remaining ones within the series of the indicia elements). In some embodiments, the varying embossing heights within such a series of indicia elements are selected so as to enable a user to recognize such indicia elements as indicia elements that are similar to one another but of varying size. In some embodiments, the indicia elements in the series have varying heights that are in a range of between 0.005 inch and 0.08 inch. In some embodiments, the indicia elements in the series have varying heights that are in a range of between 0.5 mm and 1.5 mm. In some embodiments, the indicia elements in the series have varying heights that are in a range of between 3 mm and 6 mm. In some embodiments, such a series of indicia elements includes indicia elements having heights of 6 mm, 5.1 mm, 4 mm, and 3.15 mm.

In some embodiments, at least one indicator 100 within a set of indicators includes a haptic signature 120 that includes an indicia element having a width that varies across a length thereof so as to define an expanding profile. In some embodiments, the indicia element has a width that varies across a range of between 0.05 inch and 0.5 inch. In some embodiments, the indicia element has a width that varies across a range of between 0.05 inch and 0.2 inch. In some embodiments, the indicia element has a width that varies across a range of between 0.2 inch and 0.35 inch. In some embodiments, the indicia element has a width that varies across a range of between 0.35 inch and 0.5 inch. In some embodiments, the indicia element has a width that varies across a range of between 0.05 inch and 0.35 inch. In some embodiments, the indicia element has a width that varies across a range of between 0.2 inch and 0.5 inch.

In some embodiments, at least one indicator 100 within a set of indicators includes a haptic signature 120 that includes an embossed portion and a debossed portion. In some embodiments, at least one indicator 100 within a set of indicators includes a haptic signature 120 that includes an embossed portion having an embossing height that is in one of the ranges noted above and a debossed portion. In some embodiments, at least one indicator 100 within a set of indicators includes a haptic signature 120 that includes an embossed portion and a debossed portion having a debossing depth that is one of the ranges noted above. In some embodiments, at least one indicator 100 within a set of indicators includes a haptic signature 120 that includes an embossed portion that is embossed to a height that is in a range of from 1.4 mm to 1.5 mm, or that is in a range of from 1.4 mm to 1.44 mm, or that is about 1.42 mm, or that is 1.42 mm, and a debossed portion that is debossed to a depth that is in a range of from 0.8 mm to 1.2 mm, or that is in a range of from 0.9 mm to 1.1 mm, or that is about 1 mm, or that is 1 mm. In some embodiments, at least one indicator 100 within a set of indicators includes a haptic signature 120 that includes an embossed portion that is embossed to a height that is in a range of from 0.8 mm to 1.2 mm, or that is in a range of from 0.9 mm to 1.1 mm, or that is about 1 mm, or that is 1 mm, and a debossed portion that is debossed to a depth that is in a range of from 0.3 mm to 0.7 mm, or that is in a range of from 0.4 mm to 0.6 mm, or that is about 0.5 mm, or that is 0.5 mm. In some embodiments, at least one indicator 100 within a set of indicators includes a haptic signature 120 that includes an embossed portion that is embossed to a height that is in a range of from 0.3 mm to 0.7 mm, or that is in a range of from 0.4 mm to 0.6 mm, or that is about 0.5 mm, or that is 0.5 mm, and a debossed portion that is debossed to a depth that is in a range of from 0.3 mm to 0.7 mm, or that is in a range of from 0.4 mm to 0.6 mm, or that is about 0.5 mm, or that is 0.5 mm.

In some embodiments, at least one indicator 100 within a set of indicators includes a haptic signature 120 that includes an embossed portion and a debossed/indented portion within the embossed portion. In some embodiments, at least one indicator 100 within a set of indicators includes a haptic signature 120 that includes an embossed portion having an embossing height that is in one of the ranges noted above and a debossed/indented portion within the embossed portion. In some embodiments, at least one indicator 100 within a set of indicators includes a haptic signature 120 that includes an embossed portion having an embossing height that is in one of the ranges noted above and a debossed/indented portion within the embossed portion having a debossing depth that is one of the ranges noted above. In some embodiments, at least one indicator 100 within a set of indicators includes a haptic signature 120 that includes an embossed portion having an embossing height that is in a range of from 1.7 mm to 2.1 mm, or that is in a range of from 1.8 mm to 2 mm, or that is about 1.9 mm, or that is 1.9 mm, and a debossed/indented portion within the embossed portion having a debossing depth that is in a range of from 1.8 mm to 2.2 mm, or that is in a range of from 1.9 mm to 2.1 mm, or that is about 2 mm, or that is 2 mm. In some embodiments, at least one indicator 100 within a set of indicators includes a haptic signature 120 that includes an embossed portion having an embossing height that is in a range of from 1.13 mm to 1.53 mm, or that is in a range of from 1.23 mm to 1.43 mm, or that is about 1.33 mm, or that is 1.33 mm, and a debossed/indented portion within the embossed portion having a debossing depth that is in a range of from 0.3 mm to 0.7 mm, or that is in a range of from 0.4 mm to 0.6 mm, or that is about 0.5 mm, or that is 0.5 mm.

In some embodiments, at least one indicator 100 within a set of indicators includes a haptic signature 120 that includes a debossed portion and an embossed portion within the debossed portion. In some embodiments, at least one indicator 100 within a set of indicators includes a haptic signature 120 that includes a debossed portion having a debossing depth that is in one of the ranges noted above and an embossed portion within the debossed portion. In some embodiments, at least one indicator 100 within a set of indicators includes a haptic signature 120 that includes a debossed portion having a debossing depth that is in one of the ranges noted above and an embossed portion within the debossed portion having an embossing height that is one of the ranges noted above. In some embodiments, at least one indicator 100 within a set of indicators includes a haptic signature 120 that includes a debossed portion having a debossing depth that is in a range of from 0.8 mm to 1.2 mm, or that is in a range of from 0.9 mm to 1.1 mm, or that is about 1 mm, or that is 1 mm, and an embossed portion within the debossed portion having an embossing height that is in a range of from 0.7 mm to 1.1 mm, or that is in a range of from 0.8 mm to 1 mm, or that is about 0.9 mm, or that is 0.9 mm.

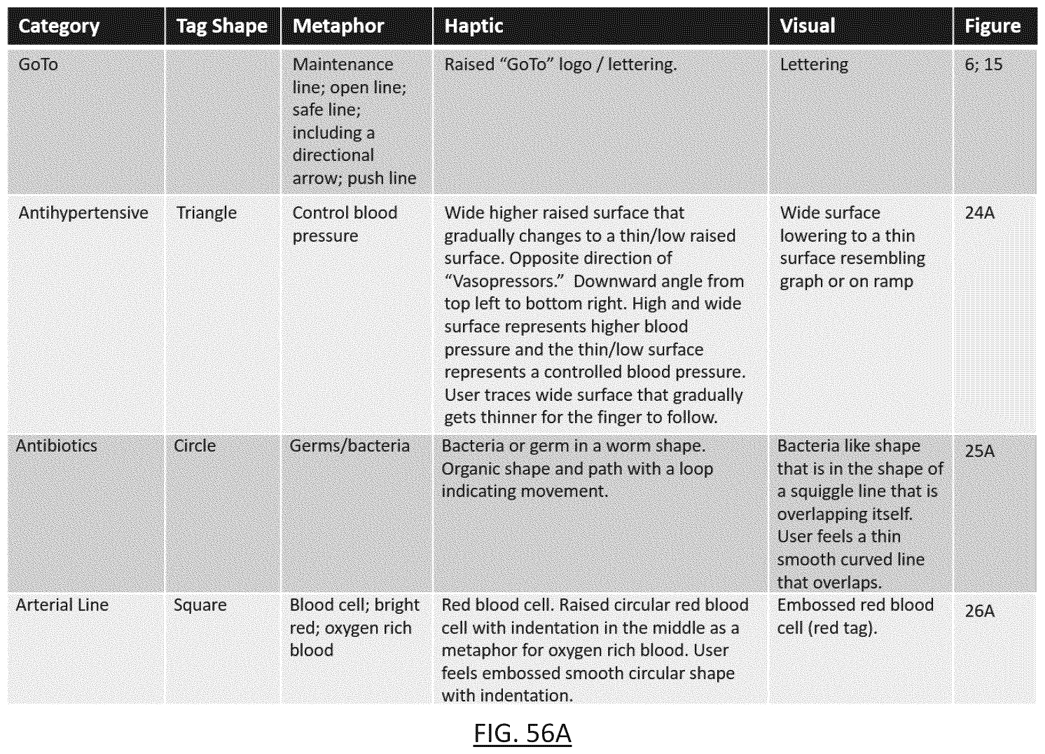

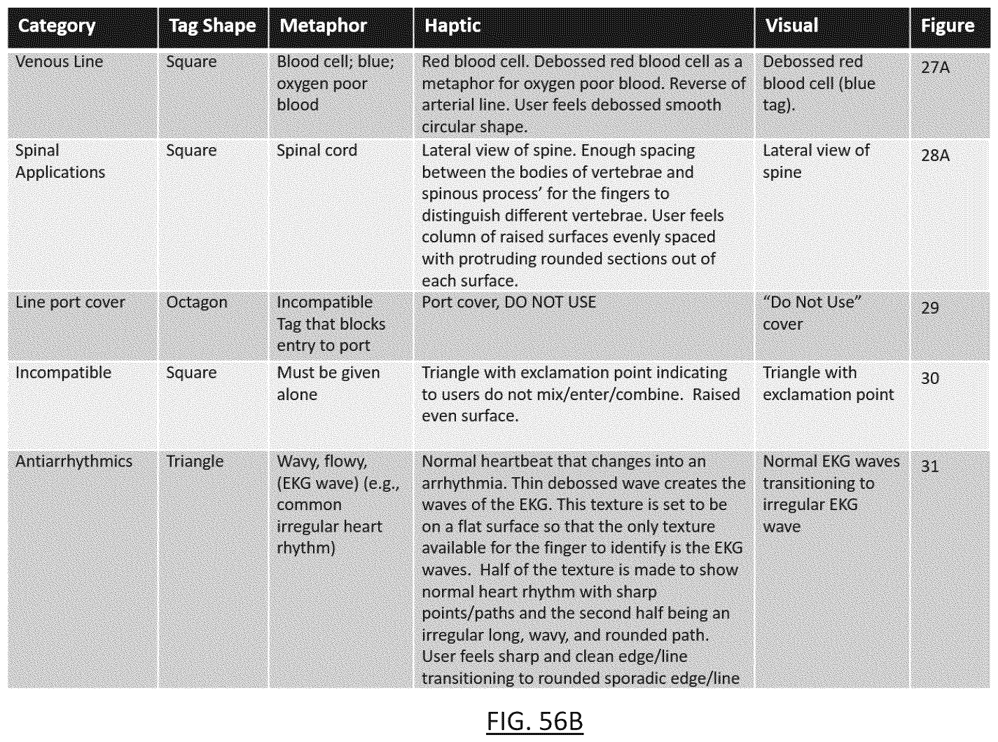

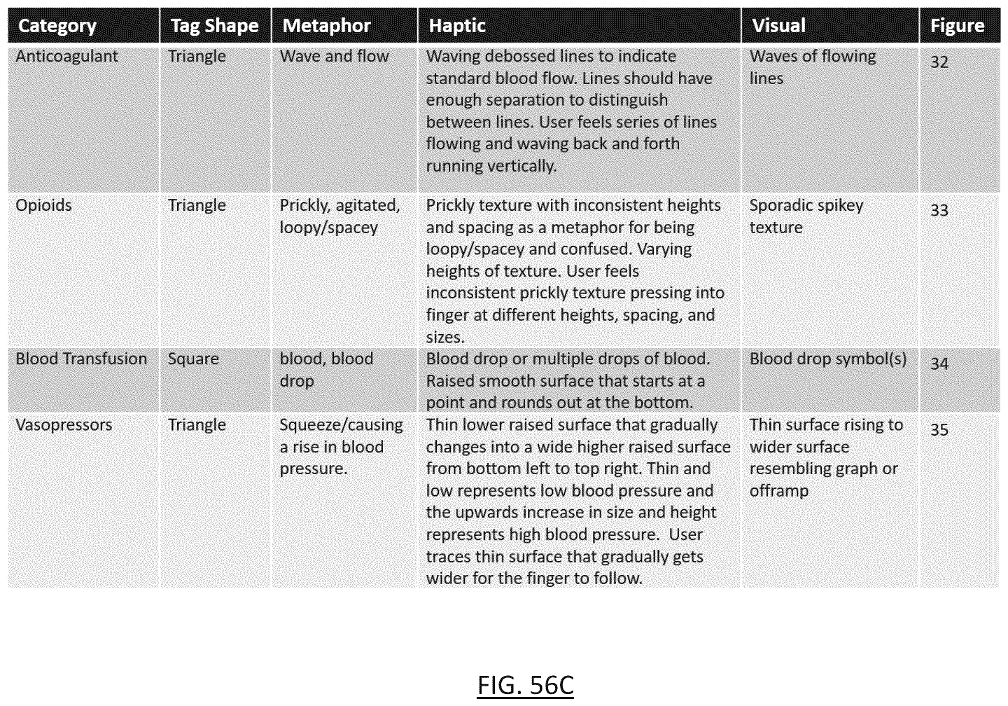

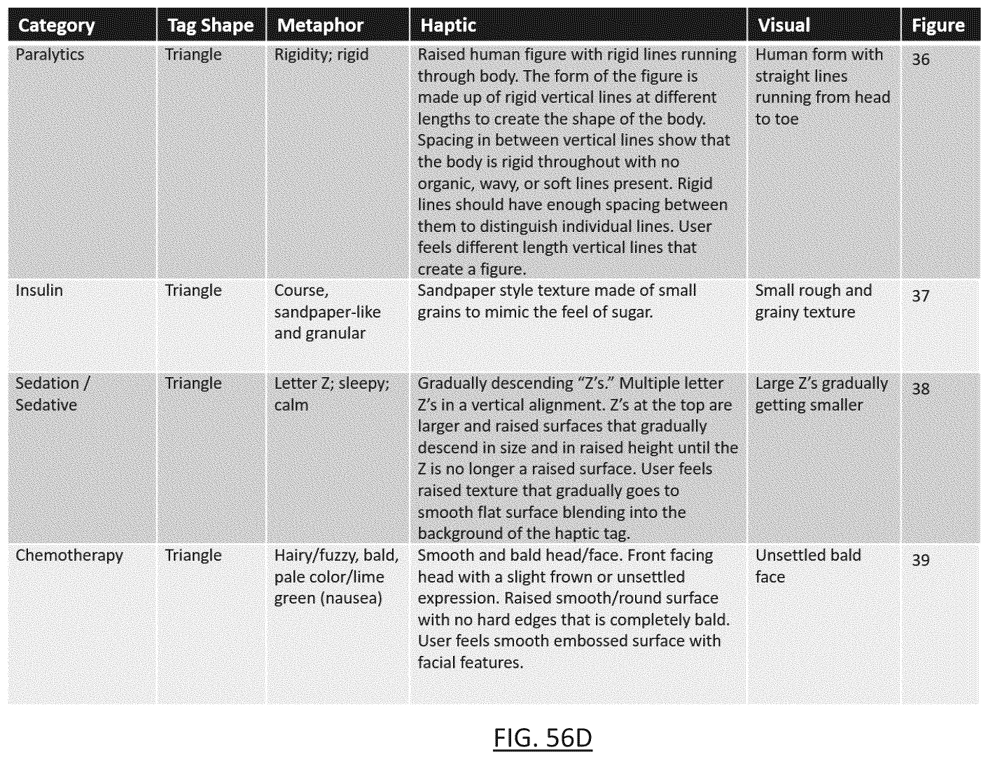

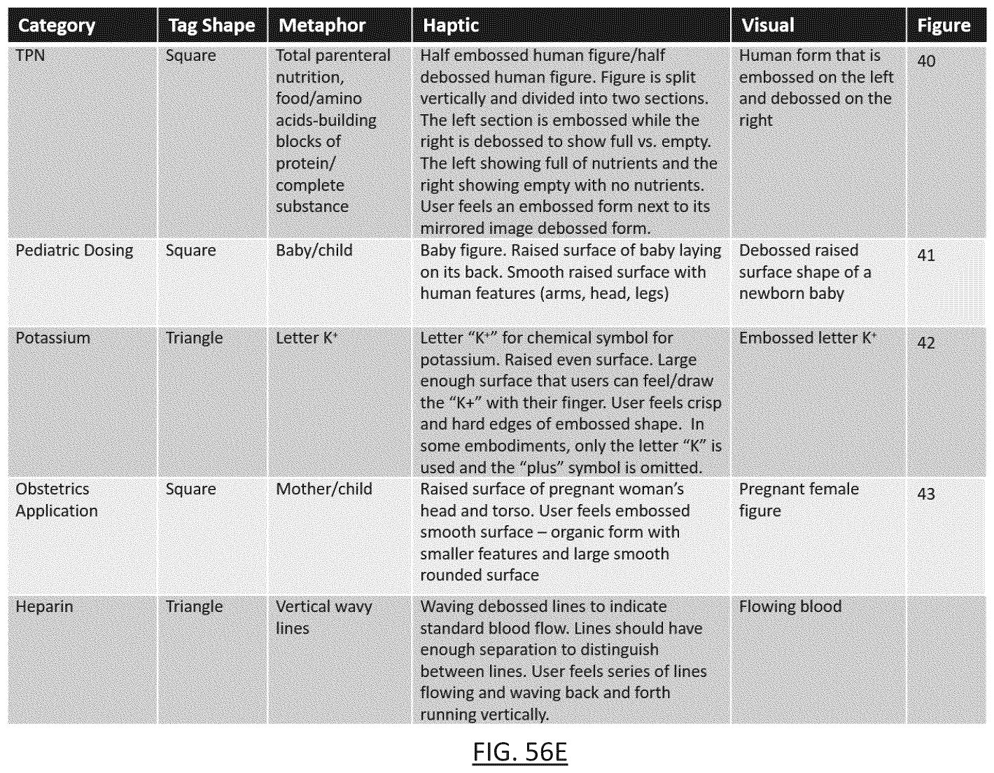

While not intended to be limiting, several examples of haptic signatures are listed in the table shown in FIGS. 56A, 56B, 56C, 56D, and 56E, along with information regarding their specific applications, metaphors, haptic and visual signatures.

As illustrated in the table shown in FIGS. 56A-56E, the haptic signatures may correspond to various classes of medications (e.g., narcotics, anticoagulants, antiarrhythmics and antibiotics), as well as specific types of applications or dosing (e.g., obstetrics and spinal applications, pediatric dosing), functions (e.g., “GoTo” maintenance line (which is further discussed below) and line port cover) or anatomical designations (e.g., arterial or venous lines). These haptic signatures are configured to be easily learned, and remembered, by the caregiver, patient and patient advocate.

In addition to the haptic signatures, each indicator 100 may have a specific color and/or shape that is associated with the various classes of medications, specific types of applications or dosing, functions or anatomical designations. Each indicator 100 may further include a specific luminosity (i.e., glowing in the dark) as a further identifying characteristic. The combination of the identifying characteristics of color, shape, luminosity, text, and haptic signature imbue each indicator 100 with a unique, multi-sensory characteristic, or communication. As discussed above, the specific combinations of multi-sensory communications alert not only the user (but also the patient, family or patient advocate) to the contents of the infusion therapy tubing (e.g., different classes or types of medications or fluids) and enable the user to take appropriate action in connection therewith. By the patient, family, patient advocate being aware of the medication in use, this provides another level of checks and balances to minimize or prevents medication-related errors that can be catastrophic, and even fatal.

In one embodiment, each indicator 100 includes at least three of the aforesaid identifying characteristics. In another embodiment, each indicator 100 includes at least four of the aforesaid identifying characteristics. In another embodiment, each indicator 100 includes at least five of the aforesaid identifying characteristics. In addition to the identifying characteristics discussed above, text (i.e., alphanumeric indicia) also constitutes one identifying characteristic in some embodiments.

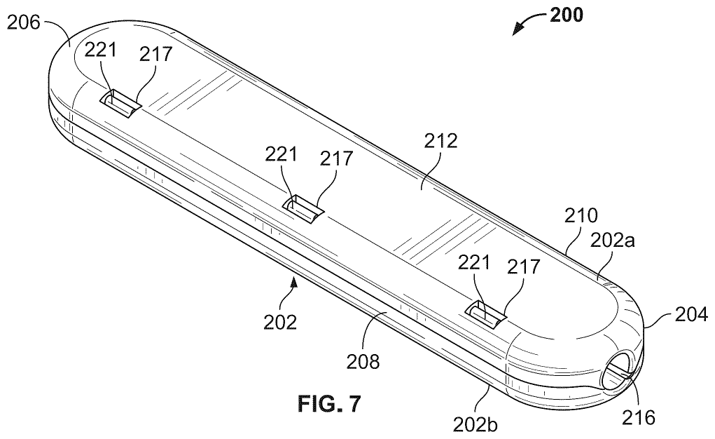

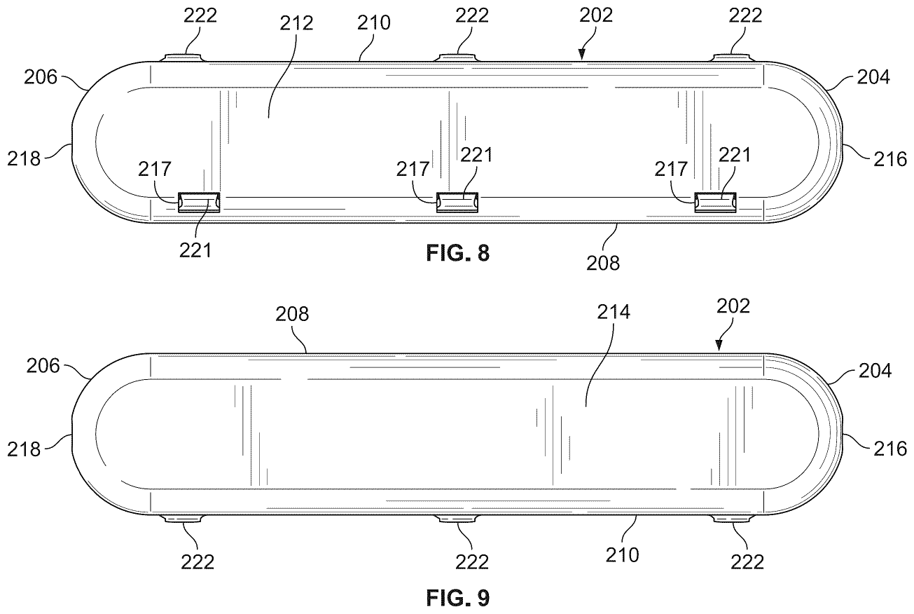

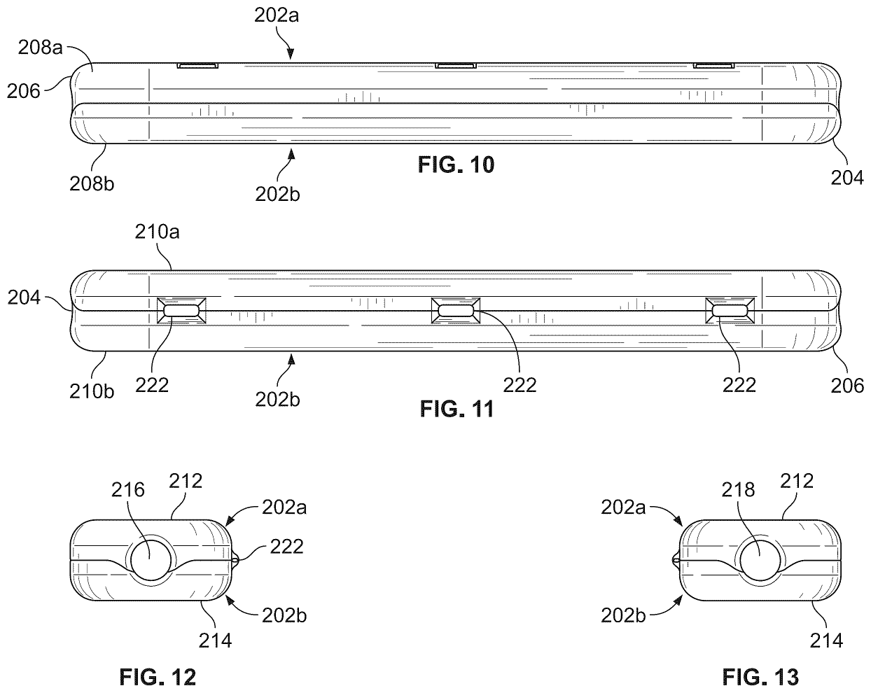

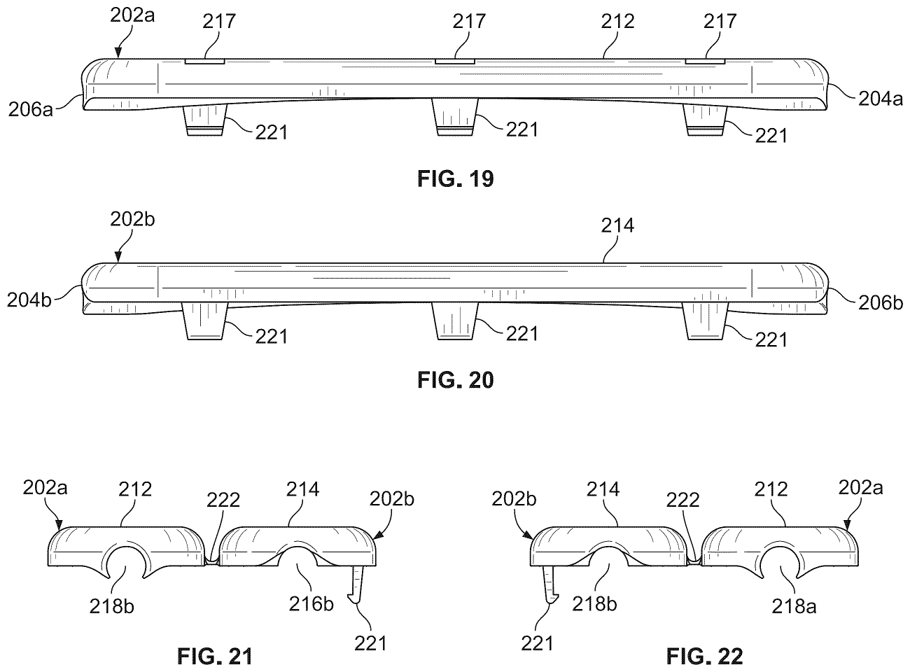

FIGS. 7-22 illustrate a second embodiment of an infusion therapy tubing indicator 200 according to the present disclosure. The indicator 200 includes a body 202 that is formed from two cooperating members 202a and 202b. Each of the members 202a, 202b has two opposed ends 204a, 206a and 204b, 206b, respectively. When the members 202a, 202b are secured together (see FIGS. 7-15), the respective ends 204a, 206a and 204b, 206b cooperate to form opposed ends 204, 206 of the body 202.

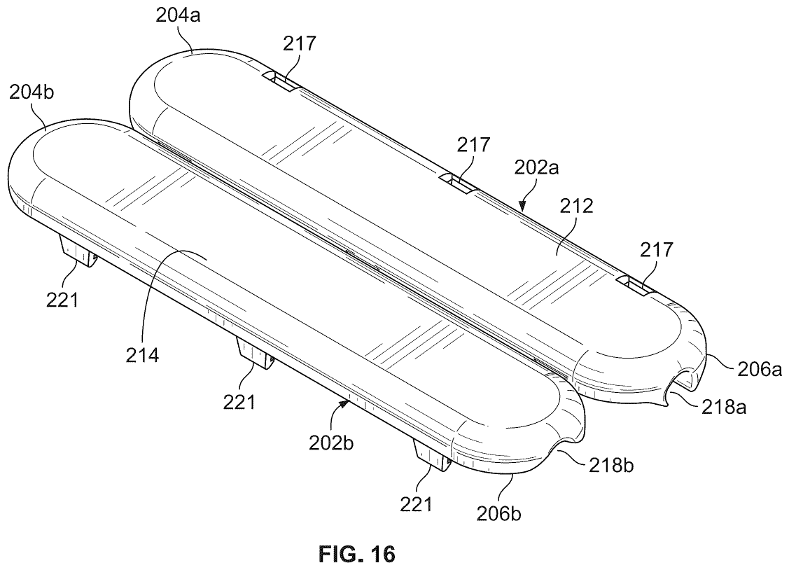

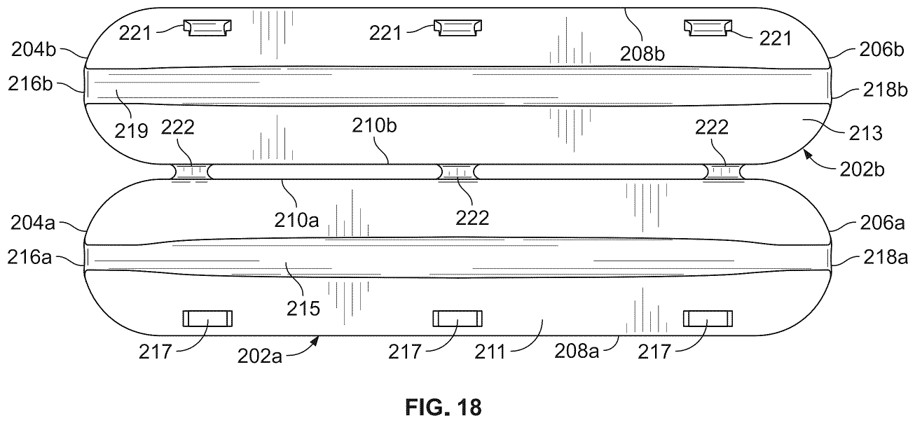

Referring now to FIGS. 16-22, the member 202a has opposed sides 208a, 210a that extend between ends 204a, 206a, and opposed inner and outer surfaces 211, 212 that extend between ends 204a, 206a and are bordered by sides 208a, 210a. The end 204a defines a partial aperture 216a therein and the end 206a defines a partial aperture 218a therein. The inner surface 211 defines a channel 215 that extends between partial apertures 216a, 218a, and is continuous therewith. The member 202a also includes one or more slots 217 formed therein, between the inner and outer surfaces 211, 212. In one exemplary embodiment, there are three slots 217 (see FIGS. 16 and 18). The channel 215 and slots 217 are discussed below.

With continued reference to FIGS. 16-22, the member 202b has a somewhat similar construction to that of the member 202a. The member 202b has opposed sides 208b, 210b that extend between ends 204b, 206b, and opposed inner and outer surfaces 213, 214 that extend between ends 204b, 206b and are bordered by sides 208b, 210b. When the members 202a, 202b are secured together (see FIGS. 7-15), the respective sides 208a, 210a and 208b, 210b cooperate to form opposed sides 208, 210 of the body 202. The end 204b defines a partial aperture 216b therein and the end 206b defines a partial aperture 218b therein. The inner surface 213 defines a channel 219 that extends between partial apertures 216b, 218b, and is continuous therewith. The inner surface 213 also includes one or more tabs 221 extending therefrom. In one exemplary embodiment, there are three tabs 221 (see FIGS. 16 and 18). The channel 219 and tabs 221 are discussed below.

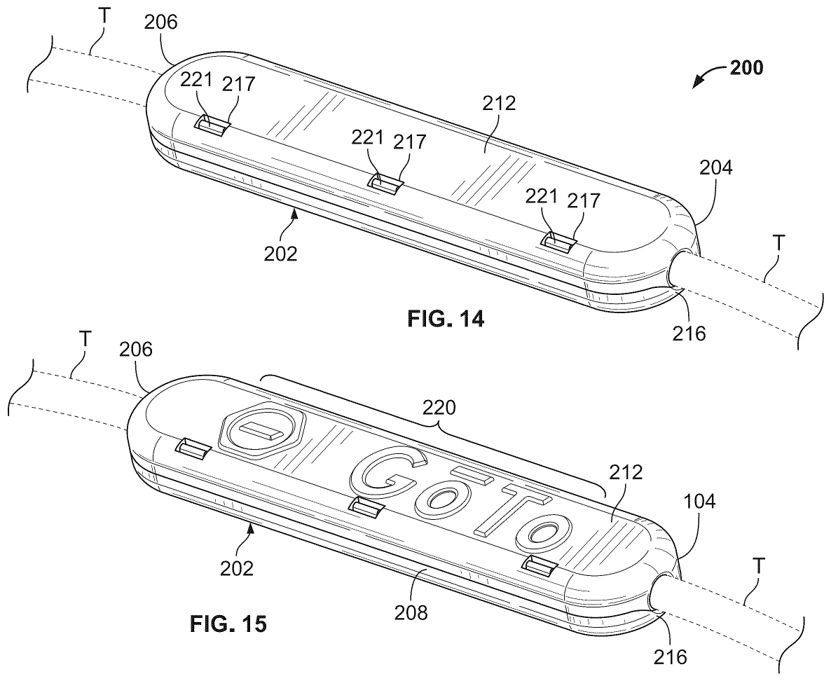

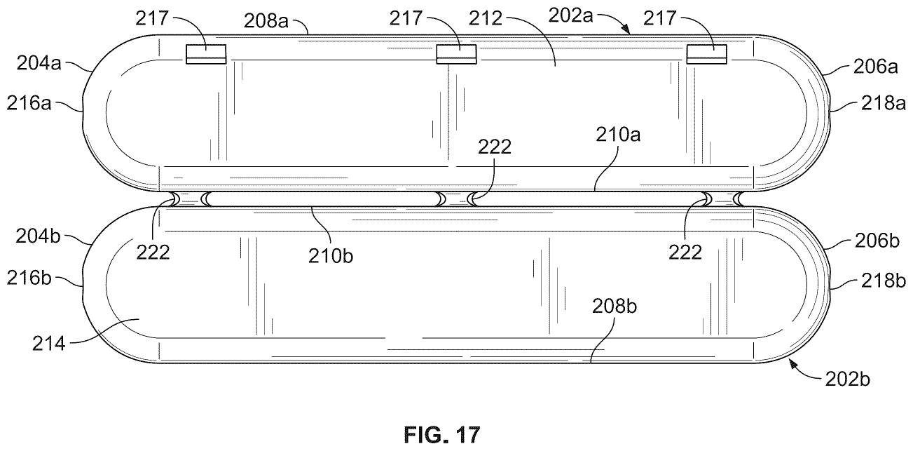

Members 202a and 202b are connected to each other by one or more hinges 222, which extend between the side 210a and 210b. In one exemplary embodiment, there are three hinges 222 (see FIGS. 17, 18, 21 and 22). The hinges 222 enable the indicator body 202 to be moved from an open to a closed position, in which members 202a and 202b fully overlap and engage one another (see FIGS. 7-15). When the body 202 is in its closed position, partial apertures 216a, 216b cooperate to form an aperture 216 at end 204, and partial apertures 218a, 218b cooperate to form an aperture 218 at end 206. The channels 215, 219 also cooperate when the body 202 is in its closed position to form a bore (not shown). The bore and apertures 216, 218 are dimensioned to receive a length of infusion therapy tubing T therethrough. More particularly, and as shown in FIGS. 14 and 15, the infusion therapy tubing T is inserted through aperture 216, extended through the bore within the interior of the indicator body 202, and exits therefrom through aperture 118 (not shown). Members 202a and 202b are secured to each other via insertion of the tabs 221 through their respective slots 217, to maintain the body 202 in its closed position. In some embodiments, the channels 215, 219 are sized and shaped so as to clasp the infusion therapy tubing T and prevent the infusion therapy tubing indicator 200 from sliding along the infusion therapy tubing T.

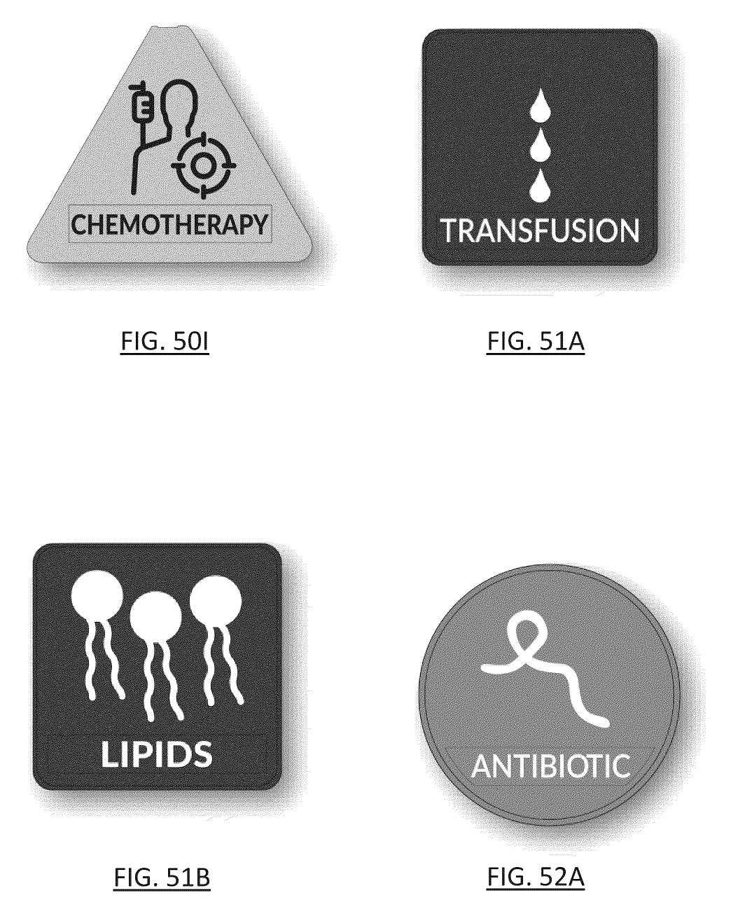

At least one of the outer surfaces 212, 214 of members 202a, 202b includes a haptic signature 220. The haptic signature 220 is similar to that of the haptic signature 120 in the first embodiment, in that it may include images, designs, numbers, letters, punctuation marks, patterns, other indicia, or any combination thereof, and is formulated to convey the nature of the medication or other substance that is contained within the infusion therapy tubing T and being delivered to the patient thereby. The haptic signature 220 is formed on the outer surface(s) 212 and/or 214 by any means known in the art, as discussed in connection with the indicator 100 above. The indicator 200 may have the same or similar haptic signatures as those set out in the table shown in FIGS. 51A-E.

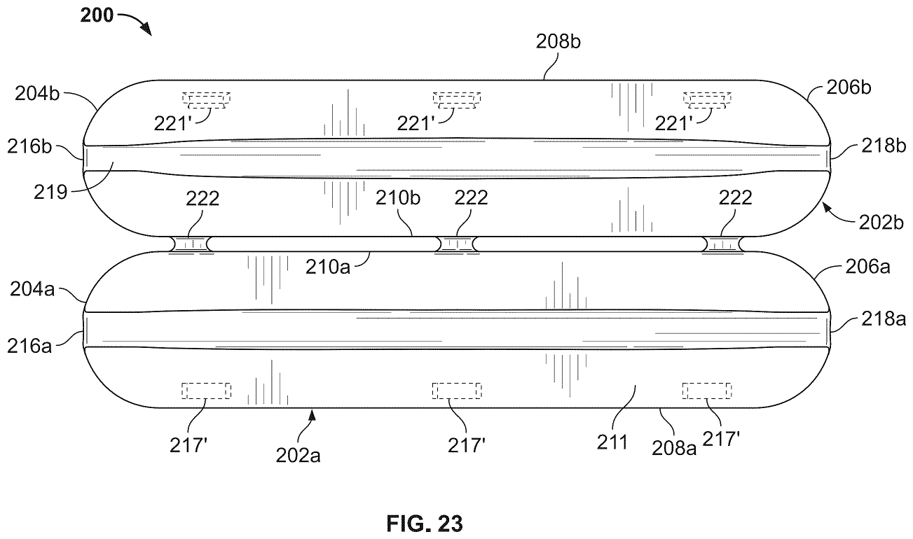

FIG. 23 illustrates another embodiment of the infusion therapy tubing indicator 200, wherein the interior fastening elements (i.e., slots 217′ and tabs 221′) can be of any suitable size and shape.

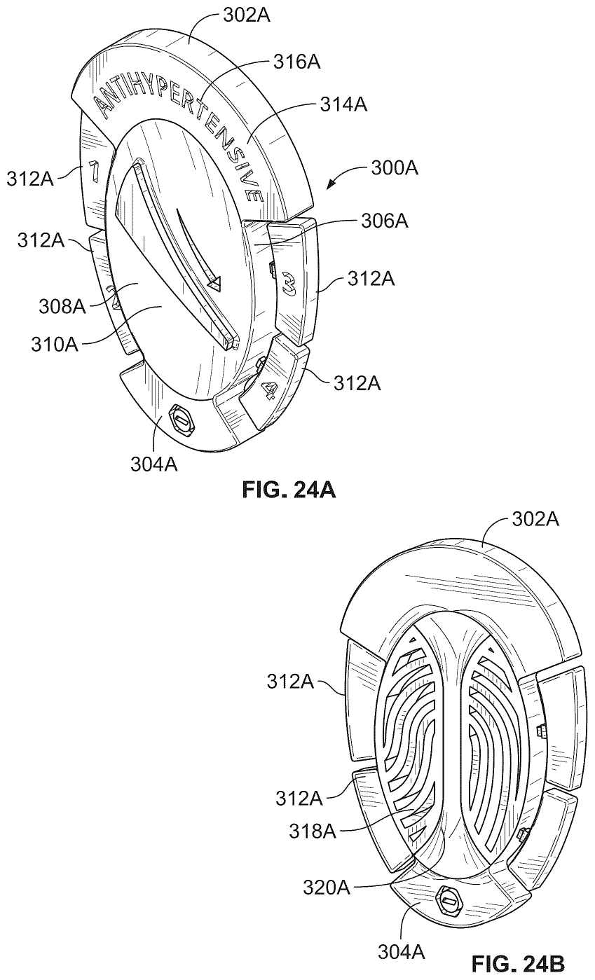

FIGS. 24A and 24B illustrate another embodiment of the infusion therapy tubing indicator 300A, which includes opposed peripheral portions 302A, 304A, a central portion 306A including a haptic signature 308A for an antihypertensive on a front surface 310A thereof, and a plurality of tabs 312A extending from the central portion 306A (as discussed above) between the opposed peripheral portions 302A, 304A. In some embodiments, the tabs 312A are numbered. In an embodiment, the tabs 312A are sequentially numbered pull or twist-off tabs to indicate the number of infusion therapy bags a patient has used during a period of time. The peripheral portion 302A includes a front surface 314A with identifying indicia 316A. FIG. 24B illustrates the rear surface 318A, in which a channel 320A is formed. The channel 320A is dimensioned to securely receive an infusion therapy line/tube therein. In some embodiments, the channel 320 is sized and shaped so as to prevent the infusion therapy tubing indicator 300A from sliding along an infusion therapy line/tube that has been received therein. In some embodiments, the infusion therapy tubing indicator 300a includes a clasp or other similar retention mechanism positioned and configured so as to facilitate retention of an infusion therapy line/tube in the channel 320A. In some embodiments, as will be described in further detail hereinafter, an indicator having the haptic signature 308A and indicia 316A indicative of an antihypertensive has an overall tag shape of a triangle and is colored red, indicating that the antihypertensive belongs to a “High Alert” category.

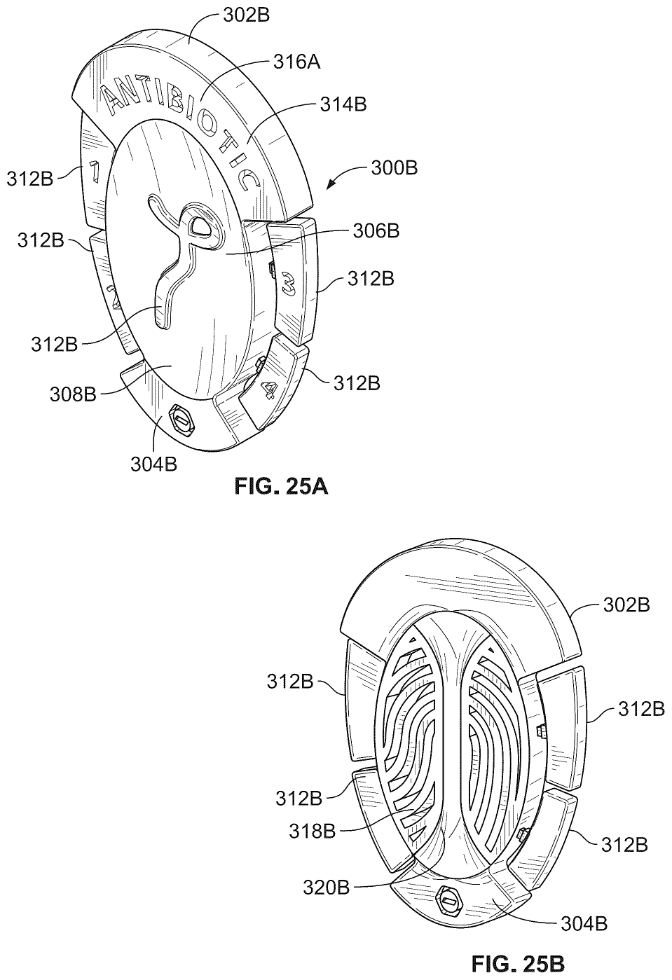

FIGS. 25A and 25B illustrate another embodiment of the infusion therapy tubing indicator 300B, which includes the same structural features as the infusion therapy tubing indicator 300A of FIGS. 24A and 24B (designated herein with a B), except for a haptic signature 308B and identifying indicia 316B for an antibiotic. In some embodiments, as will be described in further detail hereinafter, an indicator having the haptic signature 308B and indicia 316B indicative of an antibiotic has an overall tag shape of a circle and is colored orange, indicating that the antibiotic belongs to a “Non-High Alert” category.

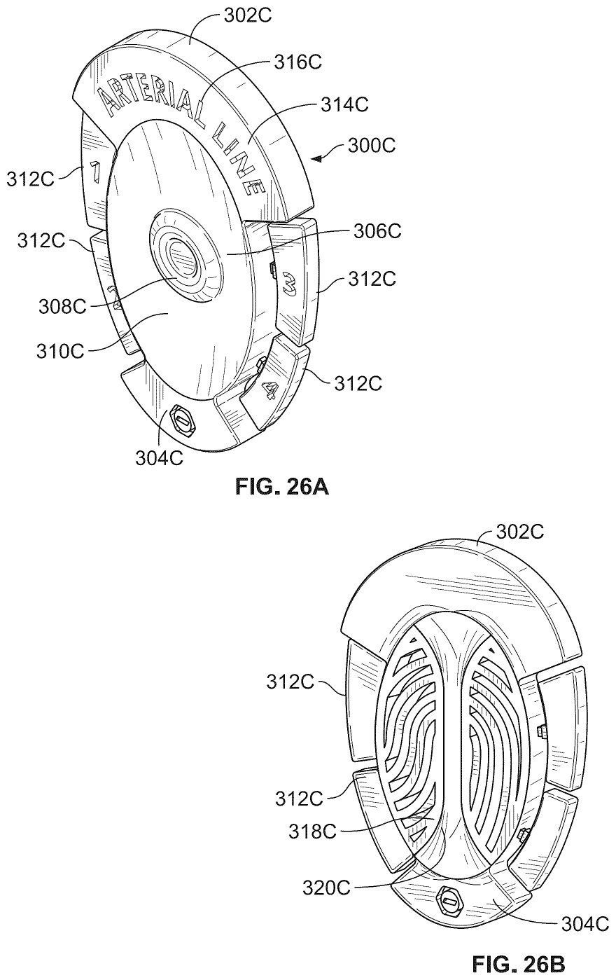

FIGS. 26A and 26B illustrate another embodiment of the tubing indicator 300C, which includes the same structural features as the infusion therapy tubing indicator 300A of FIGS. 24A and 24B (designated herein with a C), except for a haptic signature 308C and identifying indicia 316C for an arterial line. In some embodiments, as will be described in further detail hereinafter, an indicator having the haptic signature 308C and indicia 316C indicative of an arterial line has an overall tag shape of a square and is colored blue, indicating that the arterial line belongs to a “Specialty” category.

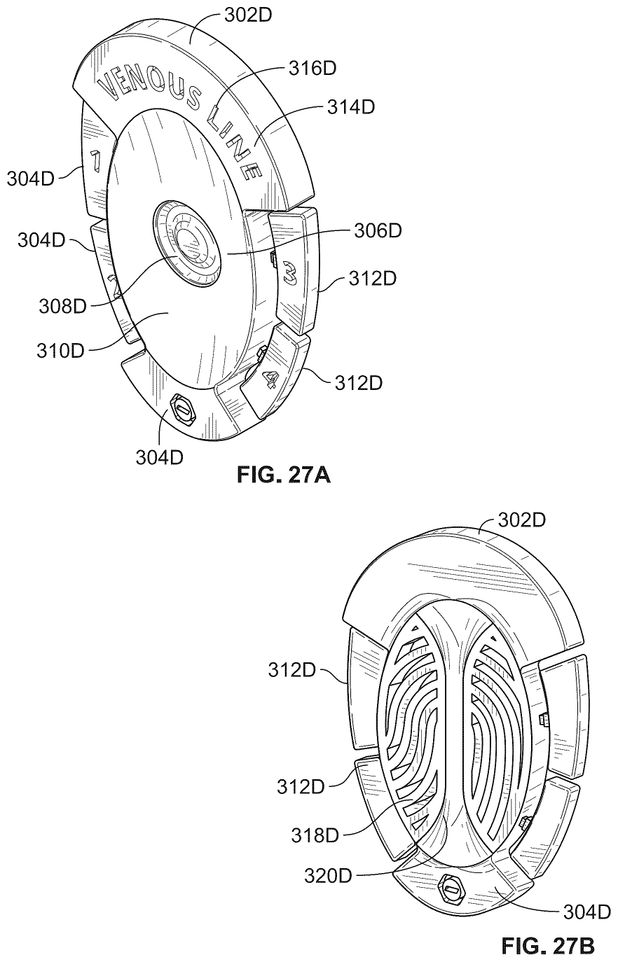

FIGS. 27A and 27B illustrate another embodiment of the tubing indicator 300D, which includes the same structural features as the infusion therapy tubing indicator 300A of FIGS. 24A and 24B (designated herein with a D), except for a haptic signature 308D and identifying indicia 316D for a venous line. In some embodiments, as will be described in further detail hereinafter, an indicator having the haptic signature 308D and indicia 316D indicative of a venous line has an overall tag shape of a square and is colored blue, indicating that the venous line belongs to a “Specialty” category.

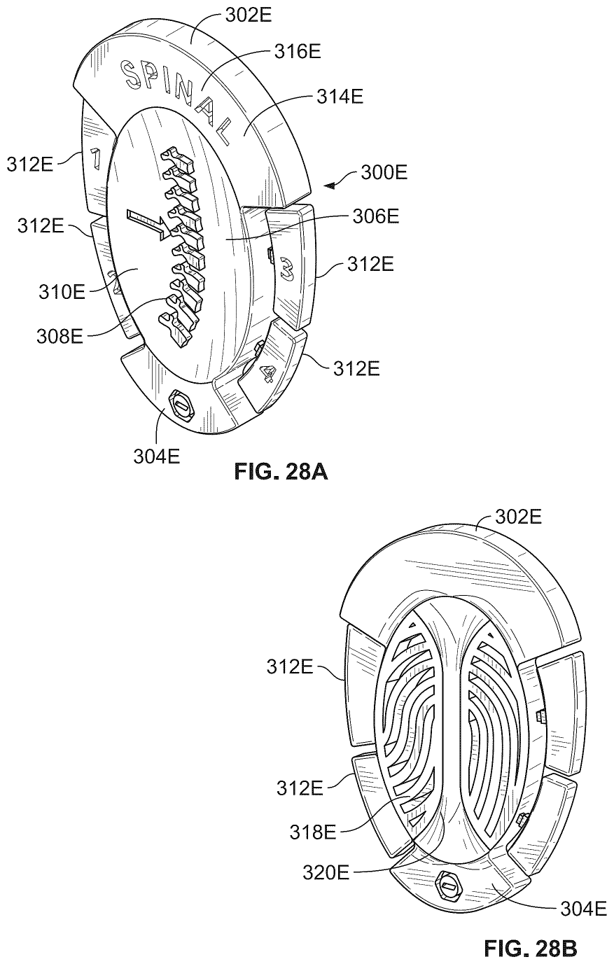

FIGS. 28A and 28B illustrate another embodiment of the infusion therapy tubing indicator 300E, which includes the same structural features as the infusion therapy tubing indicator 300A of FIGS. 24A and 24B (designated herein with an E), except for a haptic signature 308E and identifying indicia 316E for spinal administration (which may also be referred to as “intrathecal administration”). In some embodiments, as will be described in further detail hereinafter, an indicator having the haptic signature 308E and indicia 316E indicative of a spinal line has an overall tag shape of a square and is colored blue, indicating that the spinal line belongs to a “Specialty” category.

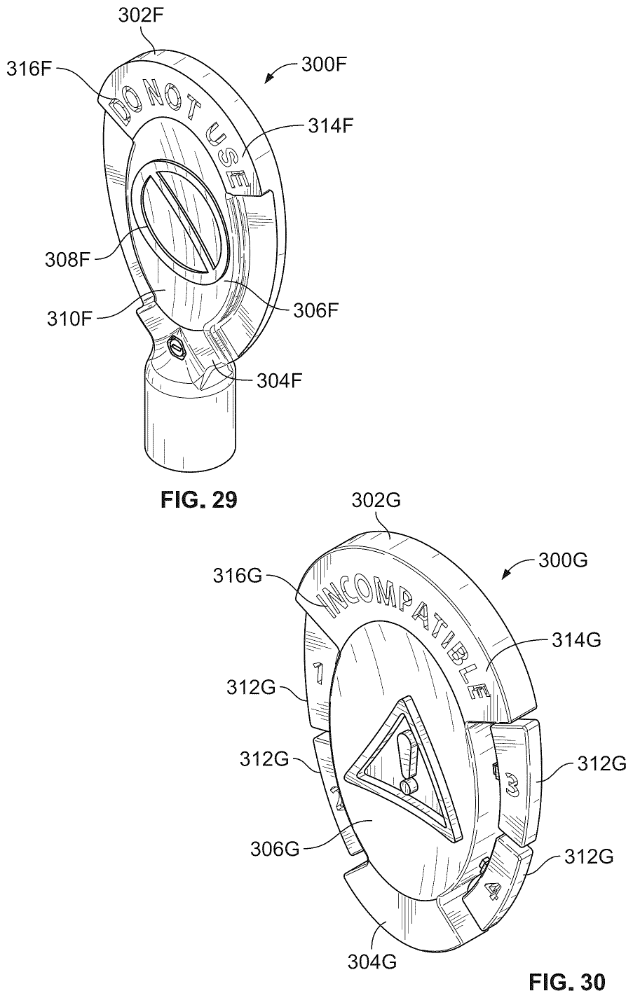

FIGS. 29-43 illustrate further embodiments of the infusion therapy tubing indicator, as discussed below. While not illustrated herein, it is to be understood that the rear surfaces of such embodiments are generally the same as those shown in FIGS. 24B, 25B, 26B, 27B and 28B, including a channel that is dimensioned to securely receive an infusion therapy line/tube therein.

FIG. 29 illustrates another embodiment of the tubing indicator 300F (e.g., for arterial, venous, or spinal lines), which includes most of the same structural features as the infusion therapy tubing indicator 300A of FIGS. 24A and 24B (designated herein with an F), except for a port cover haptic signature 308F and Do Not Use identifying indicia 316F. In an embodiment, the tubing indicator 300F also lacks tabs. In some embodiments, the infusion therapy tubing indicator 300F includes an attachment portion that is sized and shaped to attach to and cover a port that is used to administer medication into an intravenous, arterial, or spinal line, thereby preventing medication from being administered into the intravenous, arterial, or spinal line. In some embodiments, a port cover tubing indicator has a shape that is suggestive of its significance in indicating that medication should not be administered into an intravenous, arterial, or spinal line to which it has been attached. For example, in some embodiments, a port cover tubing indicator has an octagonal “stop sign” shape.

FIG. 30 illustrates another embodiment of the infusion therapy tubing indicator 300G, which includes most of the same structural features as the tubing indicator 300A of FIGS. 24A and 24B (designated herein with a G), except for an incompatible haptic signature 308G and identifying indicia 316G. In some embodiments, as will be described in further detail hereinafter, an indicator having the haptic signature 308G and indicia 316G indicative of an incompatible line has an overall tag shape of a square and is colored blue, indicating that the incompatible line belongs to a “Specialty” category.

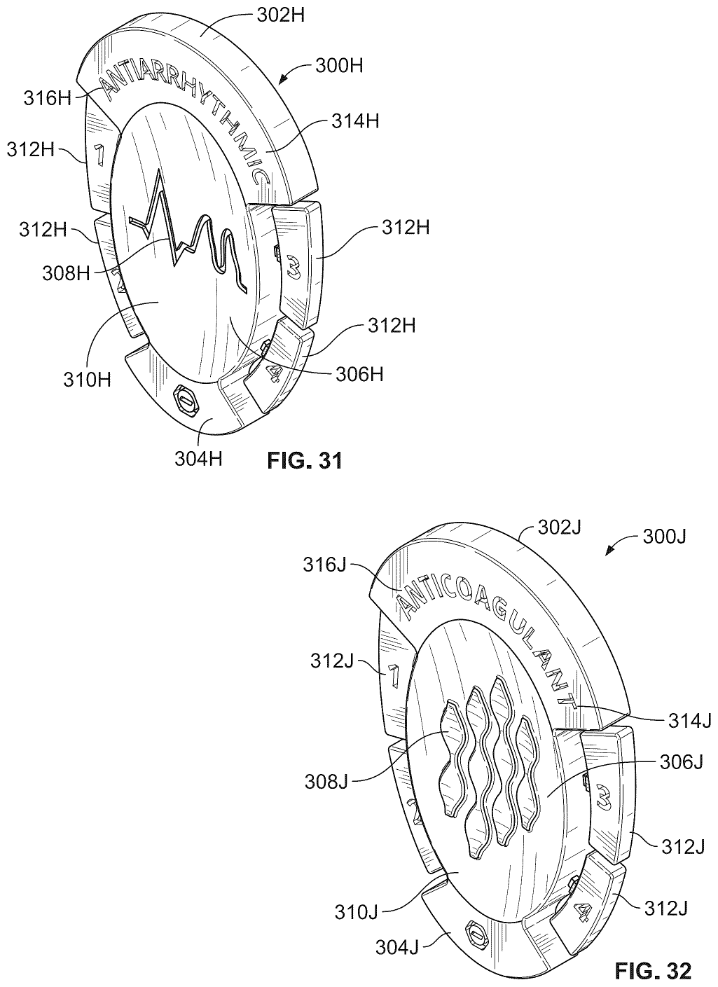

FIG. 31 illustrates another embodiment of the infusion therapy tubing indicator 300H, which includes most of the same structural features as the infusion therapy tubing indicator 300A of FIGS. 24A and 24B (designated herein with a H), except for an antiarrhythmic haptic signature 308H and identifying indicia 316H. In some embodiments, as will be described in further detail hereinafter, an indicator having the haptic signature 308H and indicia 316H indicative of an antiarrhythmic has an overall tag shape of a triangle and is colored red, indicating that the antiarrhythmic belongs to a “High Alert” category.

FIG. 32 illustrates another embodiment of the infusion therapy tubing indicator 300J, which includes most of the same structural features as the infusion therapy tubing indicator 300A of FIGS. 24A and 24B (designated herein with a G), except for an anticoagulant haptic signature 308J and identifying indicia 316J. In some embodiments, as will be described in further detail hereinafter, an indicator having the haptic signature 308J and indicia 316J indicative of an anticoagulant has an overall tag shape of a triangle and is colored red, indicating that the anticoagulant belongs to a “High Alert” category.

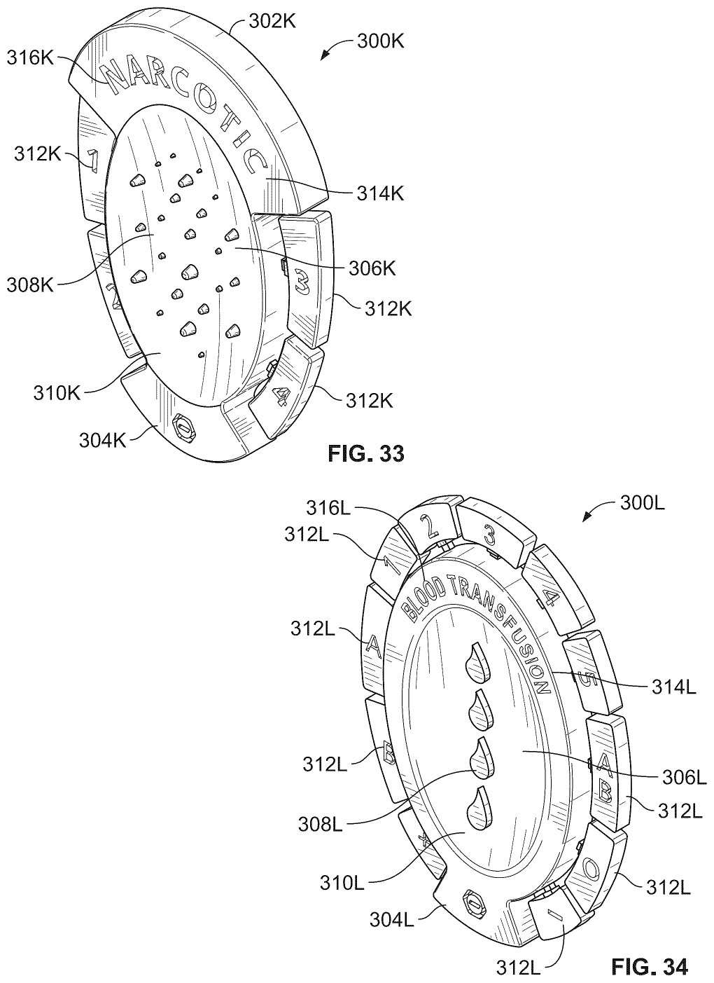

FIG. 33 illustrates another embodiment of the infusion therapy tubing indicator 300K, which includes most of the same structural features as the infusion therapy tubing indicator 300A of FIGS. 24A and 24B (designated herein with a K), except for an opioid (e.g., narcotic) haptic signature 308K and identifying indicia 316K. In some embodiments, as will be described in further detail hereinafter, an indicator having the haptic signature 308K and indicia 316K indicative of an opioid has an overall tag shape of a triangle and is colored red, indicating that the opioid belongs to a “High Alert” category.

FIG. 34 illustrates another embodiment of the infusion therapy tubing indicator 300L, which includes most of the same structural features as the infusion therapy tubing indicator 300A of FIGS. 24A and 24B (designated herein with an L), except for a blood transfusion haptic signature 308L and identifying indicia 316L. In some embodiments in which tubing indicators include tags, the infusion therapy tubing indicator 300L also includes more tabs than other embodiments. In some embodiments, as will be described in further detail hereinafter, an indicator having the haptic signature 308L and indicia 316L indicative of a transfusion has an overall tag shape of a square and is colored blue, indicating that the transfusion belongs to a “Specialty” category.

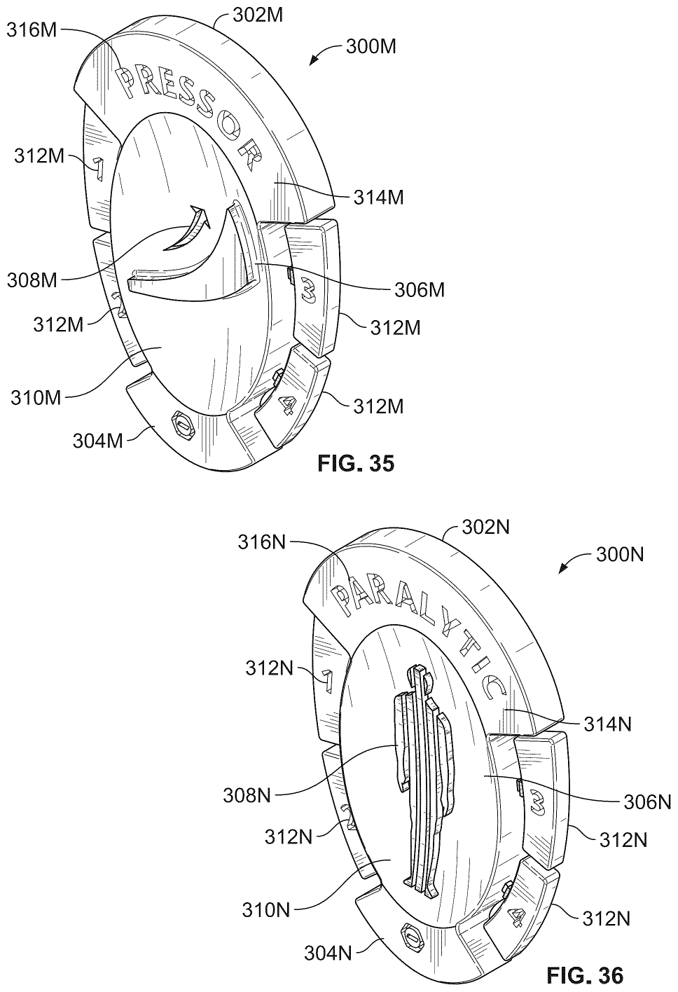

FIG. 35 illustrates another embodiment of the infusion therapy tubing indicator 300M, which includes most of the same structural features as the infusion therapy tubing indicator 300A of FIGS. 24A and 24B (designated herein with an M), except for a vasopressor haptic signature 308M and identifying indicia 316M. In some embodiments, as will be described in further detail hereinafter, an indicator having the haptic signature 308M and indicia 316M indicative of a vasopressor has an overall tag shape of a triangle and is colored red, indicating that the vasopressor belongs to a “High Alert” category.

FIG. 36 illustrates another embodiment of the infusion therapy tubing indicator 300N, which includes most of the same structural features as the infusion therapy tubing indicator 300A of FIGS. 24A and 24B (designated herein with an N), except for a paralytic haptic signature 308N and identifying indicia 316N. In some embodiments, as will be described in further detail hereinafter, an indicator having the haptic signature 308N and indicia 316N indicative of a paralytic has an overall tag shape of a triangle and is colored red, indicating that the paralytic belongs to a “High Alert” category.

FIG. 37 illustrates another embodiment of the infusion therapy tubing indicator 300P, which includes most of the same structural features as the infusion therapy tubing indicator 300A of FIGS. 24A and 24B (designated herein with a P), except for an insulin haptic signature 308P and identifying indicia 316P. In some embodiments in which some indicator tags include tabs, the infusion therapy tubing indicator 300P lacks tabs, as discussed above. In some embodiments, as will be described in further detail hereinafter, an indicator having the haptic signature 308P and indicia 316P indicative of insulin has an overall tag shape of a triangle and is colored red, indicating that the insulin belongs to a “High Alert” category.

FIG. 38 illustrates another embodiment of the infusion therapy tubing indicator 300Q, which includes most of the same structural features as the infusion therapy tubing indicator 300A of FIGS. 24A and 24B (designated herein with a Q), except for a sedative haptic signature 308Q and identifying indicia 316Q. In some embodiments, as will be described in further detail hereinafter, an indicator having the haptic signature 308Q and indicia 316Q indicative of a sedative has an overall tag shape of a triangle and is colored red, indicating that the sedative belongs to a “High Alert” category.

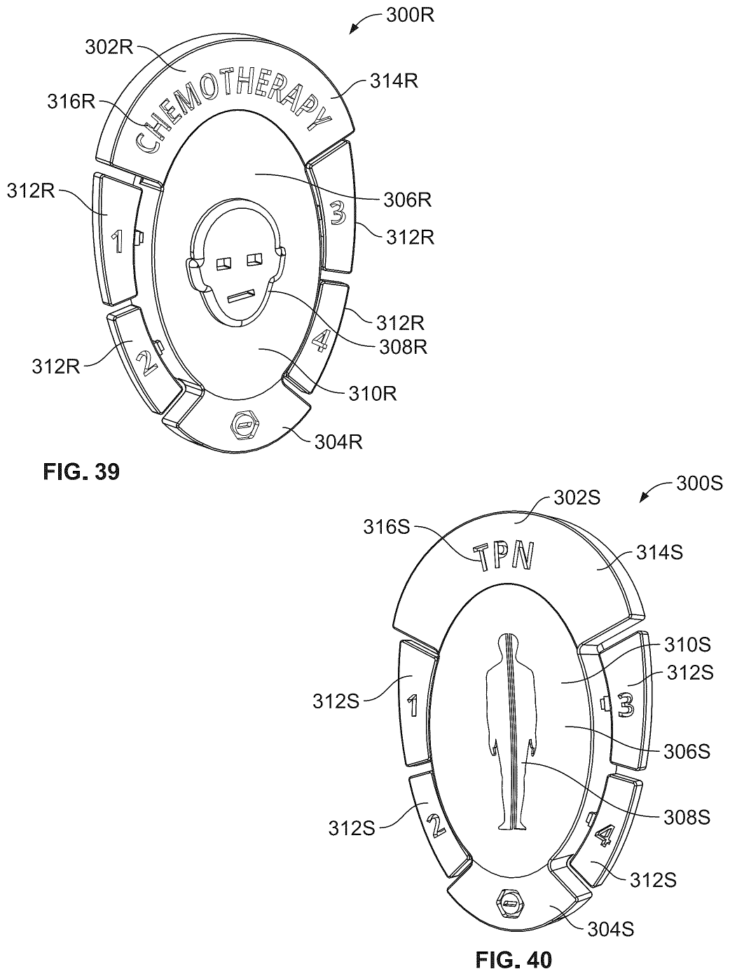

FIG. 39 illustrates another embodiment of the infusion therapy tubing indicator 300R, which includes most of the same structural features as the infusion therapy tubing indicator 300A of FIGS. 24A and 24B (designated herein with an R), except for a chemotherapy haptic signature 308R and identifying indicia 316R. In some embodiments, as will be described in further detail hereinafter, an indicator having the haptic signature 308R and indicia 316R indicative of chemotherapy has an overall tag shape of a triangle and is colored red, indicating that the chemotherapy belongs to a “High Alert” category.

FIG. 40 illustrates another embodiment of the infusion therapy tubing indicator 300S, which includes most of the same structural features as the infusion therapy tubing indicator 300A of FIGS. 24A and 24B (designated herein with a S), except for a total parenteral nutrition (TPN) haptic signature 308S and identifying indicia 316S. In some embodiments, as will be described in further detail hereinafter, an indicator having the haptic signature 308S and indicia 316S indicative of TPN has an overall tag shape of a square and is colored blue, indicating that the TPN belongs to a “Specialty” category.

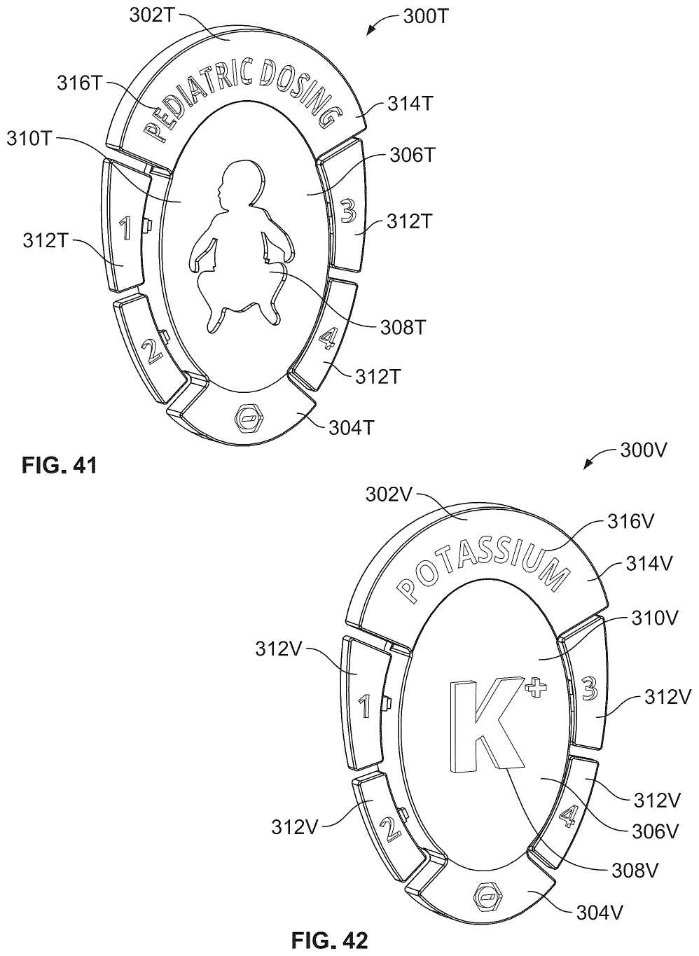

FIG. 41 illustrates another embodiment of the infusion therapy tubing indicator 300T, which includes most of the same structural features as the infusion therapy tubing indicator 300A of FIGS. 24A and 24B (designated herein with a T), except for a pediatric dosing haptic signature 308T and identifying indicia 316T. In some embodiments, as will be described in further detail hereinafter, an indicator having the haptic signature 308T and indicia 316T indicative of pediatric dosing has an overall tag shape of a square and is colored blue, indicating that the pediatric dosing belongs to a “Specialty” category.

FIG. 42 illustrates another embodiment of the infusion therapy tubing indicator 300V, which includes most of the same structural features as the infusion therapy tubing indicator 300A of FIGS. 24A and 24B (designated herein with a VP), except for a K⁺ haptic signature 308V and potassium identifying indicia 316V. In some embodiments, as will be described in further detail hereinafter, an indicator having the haptic signature 308V and indicia 316V indicative of potassium has an overall tag shape of a triangle and is colored red, indicating that the potassium belongs to a “High Alert” category.

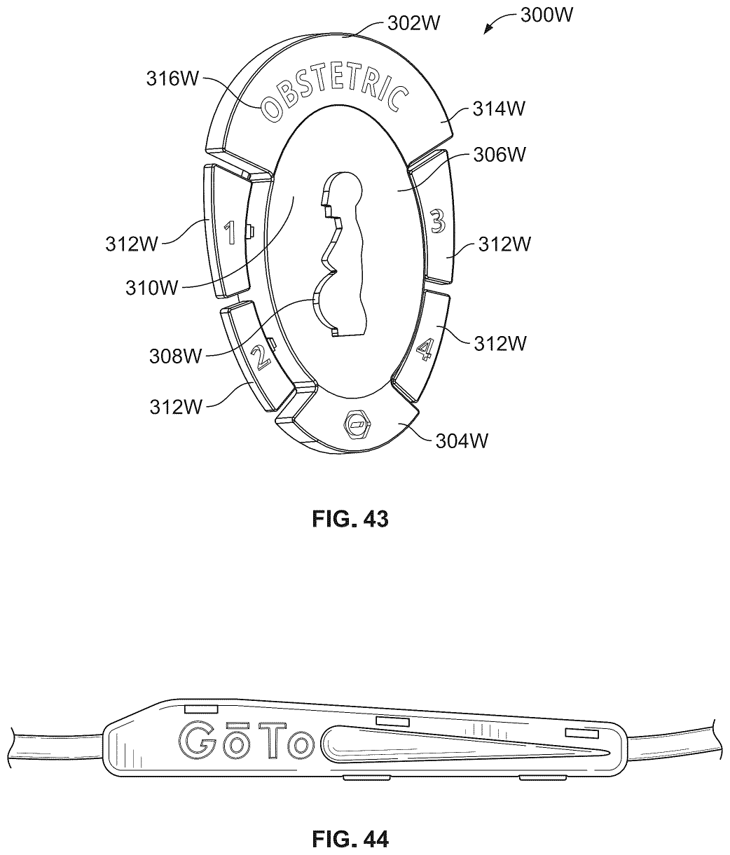

FIG. 43 illustrates another embodiment of the infusion therapy tubing indicator 300W, which includes most of the same structural features as the infusion therapy tubing indicator 300A of FIGS. 24A and 24B (designated herein with a W), except for an obstetric haptic signature 308W and identifying indicia 316W. In some embodiments, as will be described in further detail hereinafter, an indicator having the haptic signature 308W and indicia 316W indicative of obstetric administration has an overall tag shape of a square and is colored blue, indicating that the obstetric administration belongs to a “Specialty” category.

In some embodiments, any of the infusion therapy tubing indicators 300A-300W shown in FIGS. 23-43 may include images, designs, numbers, letters, punctuation marks, colors, shapes, patterns, other indicia, or any combination thereof, and is formulated to convey the nature of the medication or other substance that is contained within infusion therapy tubing to which the respective one of the infusion therapy tubing indicators 300A-300W is attached and being delivered to the patient thereby. In some embodiments, an image is oriented vertically along one of the infusion therapy tubing indicators 300A-300W (e.g., oriented generally along a long axis defined between, for example, the opposed peripheral portions 302A-302W and 304A-304W). In some embodiments, an image is oriented diagonally (e.g., offset from such a long axis).

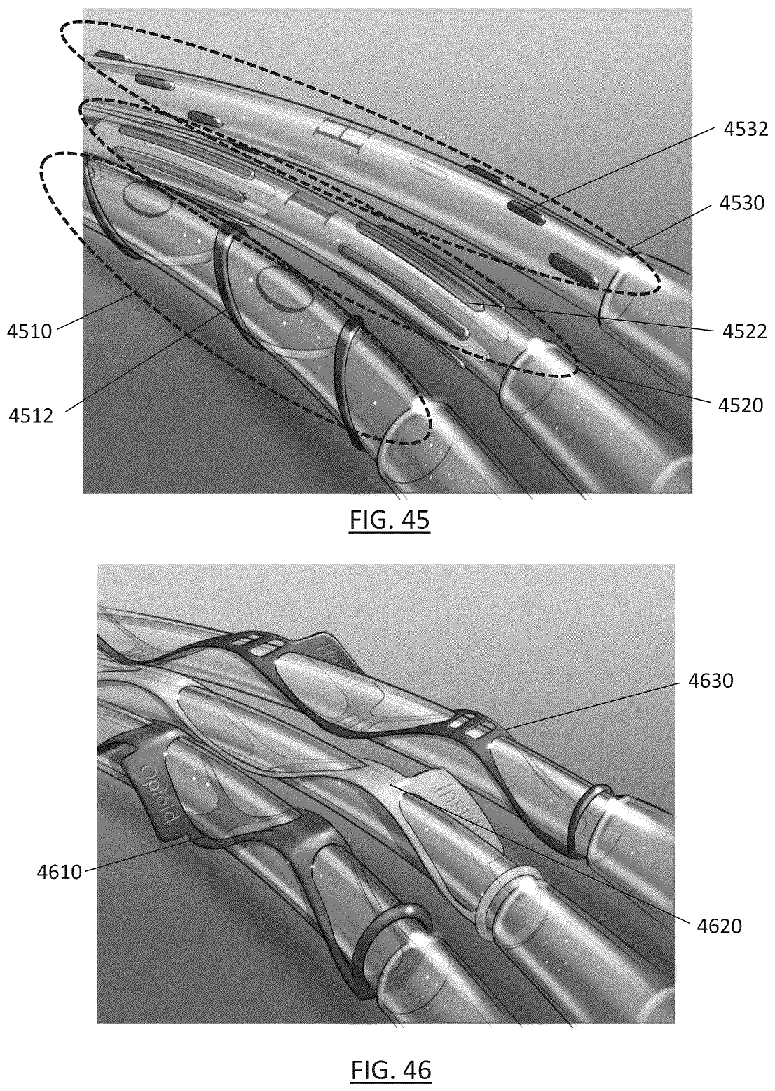

In some embodiments, an infusion therapy tubing indicator includes one or more elongate elements that are positioned along the length of an infusion therapy tube. In some embodiments, such elongate elements are fabricated by extrusion. In some embodiments, an infusion therapy tubing indicator configured to be affixed to an infusion therapy tube includes one or more discrete elongate elements, and the size, shape, and positioning of the one or more elements will vary depending on the medicament to which the infusion therapy tubing indicator corresponds. FIG. 45 illustrates embodiments of such an infusion therapy tubing indicator. A first infusion therapy tubing indicator 4510 includes an elongate element 4512 that extends along an infusion therapy tube in a spiral/helical orientation. A second infusion therapy tubing indicator 4520 includes a plurality of elongate elements 4522 that extend along an infusion therapy tube. In some embodiments, each of the elongate elements 4522 extends substantially longitudinally along the infusion therapy tube. In some embodiments, the elongate elements 4522 are divided into groups of the elongate elements 4522 that are aligned longitudinally along the infusion therapy tube and spaced apart circumferentially around the infusion therapy tube. In some embodiments, the groups of the elongate elements 4522 are spaced apart longitudinally along the infusion therapy tube. A third infusion therapy tubing indicator 4530 includes a plurality of elongate elements 4532 that extend along an infusion therapy tube. In some embodiments, each of the elongate elements 4532 extends longitudinally along the infusion therapy tube. In some embodiments, adjacent ones of the elongate elements 4532 are offset from one another both longitudinally along the infusion therapy tube and circumferentially around the perimeter of the infusion therapy tube, such that the elongate elements 4532 provide a helical arrangement around and along the infusion therapy tube. In some embodiments, the infusion therapy tubing indicators 4510, 4520, 4530 are color-coded to indicate the contents of the infusion therapy tubes to which they are joined. In some embodiments, the infusion therapy tubing indicators 4510, 4520, 4530 are used in connection with text labels to indicate the contents of the infusion therapy tubes to which they are joined.

In some embodiments, an infusion therapy tubing indicator includes a threaded sheath that extends along an infusion therapy tube. FIG. 46 illustrate embodiments 4610, 4620, 4630 of such an infusion therapy tubing indicator. In some embodiments, the each of the infusion therapy tubing indicators 4610, 4620, 4630 travels back and forth from one side of a corresponding infusion therapy tube to another in a “zigzag” shape. In some embodiments, the specific shape of each of the infusion therapy tubing indicators (e.g., spacing between each repetition, contour at each side of the infusion therapy tube, etc.) varies from one of the infusion therapy tubing indicators 4610, 4620, 4630 to another. In some embodiments, the infusion therapy tubing indicators 4610, 4620, 4630 are color-coded to indicate the contents of the infusion therapy tubes to which they are joined. In some embodiments, the infusion therapy tubing indicators 4610, 4620, 4630 include text labels to indicate the contents of the infusion therapy tubes to which they are joined.

Referring again to FIGS. 6 and 15, these illustrate embodiments of a “go-to line”, which is the line where emergency medication is injected. The go-to line (depicted in the figures and represented herein as the “GoTo Line”) must have an access port for the administration of emergency medication and it must satisfy all the requirements for the safe administration of any emergency medication.

In some embodiments, the disclosed system employs identification tags (i.e., indicators 100 and 200) that are distributed along the GoTo Line to clearly designate the appropriate line for administration of emergency or as-needed infusion medication.

Sometimes multiple doses of medication are injected or infused during an emergency at different times during a crisis. On many occasions after stabilizing the patient, the crisis recurs sometime later, at which time the GoTo line and other haptic indicators will still be immediately recognizable, making the line available for use. For example, multiples of a single type of medicament-specific haptic indicator (e.g., the indicator 300A of FIGS. 24A and 24B, the indicator 300B of FIGS. 25A and 25B, etc.) may remain present on a single infusion therapy line, indicating that such infusion therapy line is available for further administration of the same medicament. In some embodiments, in which such haptic indicators include twist-off tabs to indicate the number of bags of an infusion therapy medicament that have been administered to a patient, each of the multiples may have a differently numbered twist-off tab removed therefrom.

When there is an emergency, such as sudden deterioration in the patient's condition, it is necessary to find the GoTo Line quickly and confidently. In some embodiments, a tag identifying the GoTo line is purposefully and substantially different in appearance from other line identification tags to facilitate rapid identification.

In one embodiment, the GoTo Line is identical to a generic infusion line except that it has three special identification tags (e.g., three instances of the indicator 100 or of the indicator 200). The tags, affixed at strategic locations along the tube, accomplish routine critical awareness of the dual role of the line: to identify and facilitate the prompt location of the line in an emergency.

In some embodiments, an exemplary indicator tag includes a first group of indicia and a second group of indicia. In some embodiments, the first group of indicia is formulated to identify an alerting level of the medication or other substance contained in an infusion therapy line to which the exemplary indicator tag is attached. For example, in some embodiments, the first group of indicia is indicative of whether the infusion therapy line carries a high-alert medication, a non-high-alert medication, a specialty medication, or an oral medication.

It is well recognized that medical care has become very sophisticated in terms of medications in all fields, surgical supportive techniques and quality of care. Nevertheless, the increasing complexity of care has not been accompanied by a decrease in medication errors. There are multiple reasons for this lack of improvement. Some of these reasons include the increased complexity of care, less time availability spent by caregivers per patient because of workload, caregiver inexperience, inadequate staffing, overburdened caregivers, stress and exhaustion, distraction, poor communication, the generic nature of infusion lines without clear distinguishing qualities, and the absence of a standard of care for infusion line labeling.

The exemplary embodiments (e.g., including tags having a first group of indicia and a second group of indicia as described herein) address these problems by passively enabling caregivers to become cognitively aware of important clinical situations and the therapy being administered to the patient. In some embodiments, this passive cognitive awareness overrides the complexity of care, workload, inexperience, distractions that may occur, and fills the gaps in communication amongst caregivers. The exemplary embodiments do so in a multitiered fashion. The first tier of awareness (e.g., a “birds-eye” view) occurs upon the caregiver entering the patient's room and without necessarily being at the patient's bedside. The first impression of different, but meaningfully shaped, tags on the infusion line (s) immediately conveys the degree of complexity of drug(s) and care that the patient is receiving.

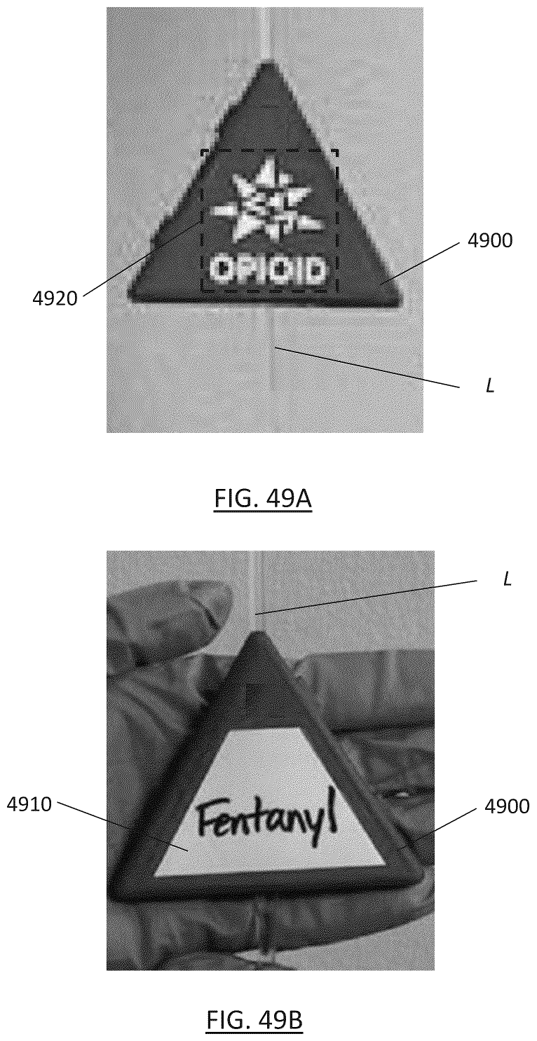

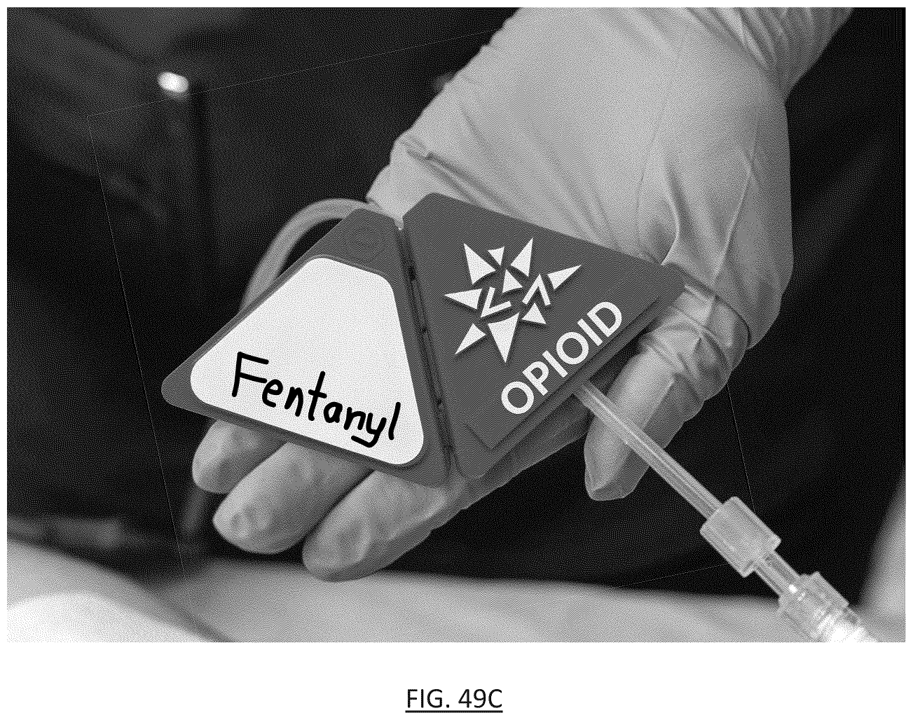

In some embodiments, a labeling tag includes a text label that is indicative of a class of therapy being delivered by the line to which the labeling tag is attached (e.g., the label “opioid” shown in FIGS. 49A-49C). In some embodiments, the labeling tag includes an icon that is indicative of a class of therapy being delivered by the line to which the labeling tag is attached. In some embodiments, the icon includes an image or other metaphoric representation of the therapy. In some embodiments, the icon includes a haptic feature (e.g., a texturing, embossing, debossing, etc.). In some embodiments, the labeling tag includes a shape that is indicative of a category of the category of therapy (e.g., high-alert, non-high-alert, specialty, etc.). For example, in the embodiment shown in FIGS. 49A-49C, the labeling tag is triangular. In some embodiments, the labeling tag includes a color that is indicative of a category of the category of therapy (e.g., high-alert, non-high-alert, specialty, etc.). For example, in the embodiment shown in FIG. 49A-49C, the labeling tag is red. In some embodiments, the labeling tag includes a writing space that is configured to allow a clinician to write information that is indicative of the therapy or other information. For example, in the embodiment shown in FIGS. 49B and 49C, the writing space has been used to write the text “Fentanyl,” which is a type of tag within the “opioid” class of therapy.

In some embodiments, a red triangle-shaped tag is used to indicate a high-alert category, an orange circle-shaped tag is used to indicate a non-high-alert category, a blue square-shaped tag is used to indicate a specialty category, and a purple diamond-shaped, oval-shaped, badge-shaped, or pentagon-shaped tag is used to indicate an oral medication category. In some embodiments, such tags are distinguishable by shape and color. In some embodiments, a second tier of information is provided by several features of the tag itself. For example, in some embodiments, in addition to the shape and color of the tag there will be a haptic feature (e.g., a physical metaphor for the drug being infused) that will inform and confirm to the caregiver the nature of the medication represented by the tag category, simply with touch (e.g., as described above). In some embodiments, the name of the medication or drug category is printed on the tag. In some embodiments, a tag includes an area upon which the caregiver may write to further characterize the medication. In some embodiments, a tag provides enhanced visibility by an illumination quality on the tag (e.g., luminosity, such as “glow-in-the-dark” type features).

In some embodiments, high-alert medications, as identified by accrediting organizations, healthcare systems, and patient safety organizations, are those that bear a heightened risk of causing significant patient harm when they are used in error or even when used as intended (such as chemotherapy medications, opioids, neuromuscular blockers, sedatives, and other types of medications). In some cases, patients may have multiple high alert medications infusing, and such tags help identify and distinguish the various high alert medications that are infusing. High-alert category tags include drugs/medications that have much less room for error on the part of the caregiver. Such drugs/medications, if provided with incorrect dosing, or when administered incorrectly, can cause serious complications. Some of these complications may include cardiac arrest, hemodynamic instability (sudden changes in blood pressure), atrial arrhythmias with the potential for stroke or heart failure, ventricular arrhythmia with the risk of cardiac arrest, respiratory failure, excessive spontaneous or postsurgical bleeding, paralysis with respiratory failure, but not necessarily with loss of consciousness. Other potential problems could include, but are not limited to, bone marrow failure, serious declines in blood sugar with associated confusion or, and respiratory suppression from narcotic use.

In some embodiments, non-high-alert tags are indicative of situations such as antibiotic therapy (e.g., antibacterial, antiviral and antifungal) or administration of antiemetics. In some embodiments, non-high-alert tags apply to the use of oral, subcutaneous, intramuscular chemotherapy or biologic medications that are in use. In some embodiments, the administration of these medications/fluids may not place the patient at immediate risk, but they nevertheless have an integral role in the management of the patient. In some embodiments, the presence or absence of these medications needs to be noted depending upon a particular clinical situation such as when the patient needs to receive antibiotics rapidly. For example, in some embodiments, the presence of the non-high alert antibiotic tag would reveal to any caregiver who enters the room, that antibiotic therapy had been initiated. Conversely, the absence of the same tag would rapidly inform the caregiver of the oversight and to initiate antibiotics immediately for a patient requiring such treatment.

In some embodiments, a specialty tag denotes obstetric care, pediatric dosing, blood product transfusions (e.g., red cells, platelets, plasma factors, whole blood), intravenous nutrition, or spinal administration.

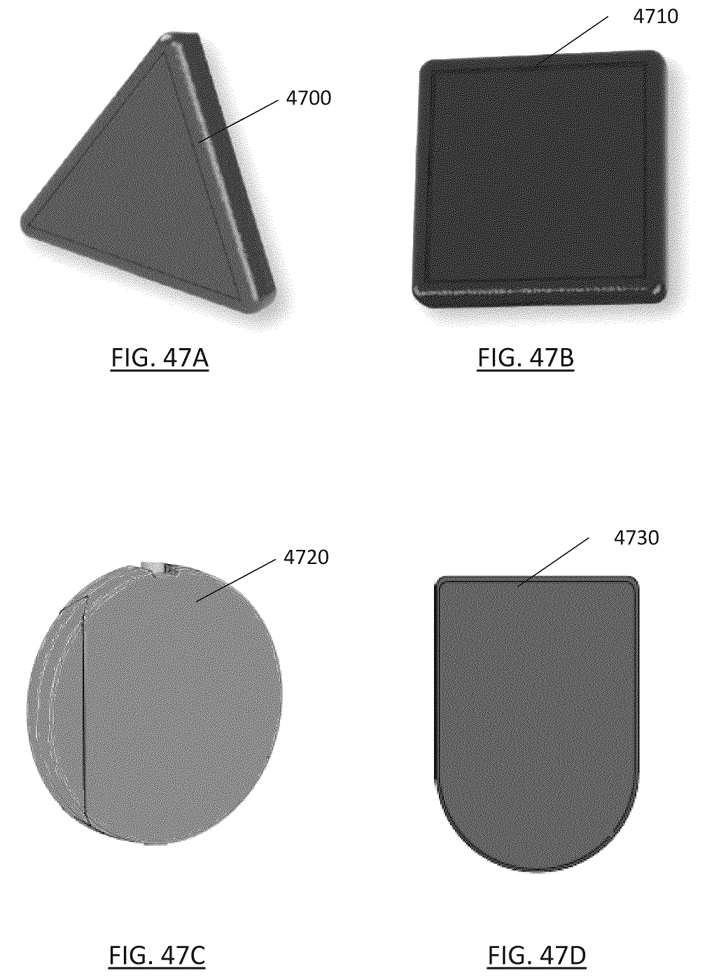

In some embodiments, the first group of indicia includes at least (1) indicator tag color and (2) indicator tag shape. For example, in some embodiments, an indicator tag of the high alert category has a generally triangular shape (e.g., triangular with angled or rounded corners) and a red color. FIG. 47A shows an exemplary high alert indicator tag 4700 having a generally triangular shape and a red color. In some embodiments, the generally triangular shape includes a flattened contour at at least one corner in order to avoid crimping of an infusion therapy line passing through the indicator tag near such an at least one corner. In some embodiments, an indicator tag of the specialty category has a generally rectangular or square shape (e.g., rectangular with angled or rounded corners, or square with angled or rounded corners) and a blue color. FIG. 47B shows an exemplary specialty indicator tag 4710 having a generally square shape and a blue color. In some embodiments, an indicator tag of the non-high alert category has a generally round shape (e.g., oval or circular) and an orange color. FIG. 47C shows an exemplary non-high alert indicator tag 4720 having a generally oval shape and an orange color. In some embodiments, an indicator tag of an oral medication category has a diamond shape, an oval shape, a badge shape, or a generally pentagonal shape, and a purple color. FIG. 47D shows an exemplary oral medication indicator tag 4730 having a badge shape and a purple color.

In some embodiments, the second group of indicia is formulated to identify to a user the nature of the medication or other substance contained in an infusion therapy line to which the exemplary indicator tag is attached. For example, in some embodiments, the second group of indicia is indicative of one of the medication types identified in the table shown in FIGS. 51A-E. In some embodiments, the second group of indicia includes at least (1) a haptic signature including a metaphor (e.g., the haptic signatures described above with reference to FIGS. 24A-43) that is suggestive of the nature of the medication or other substance contained in the infusion therapy and (2) a text label.

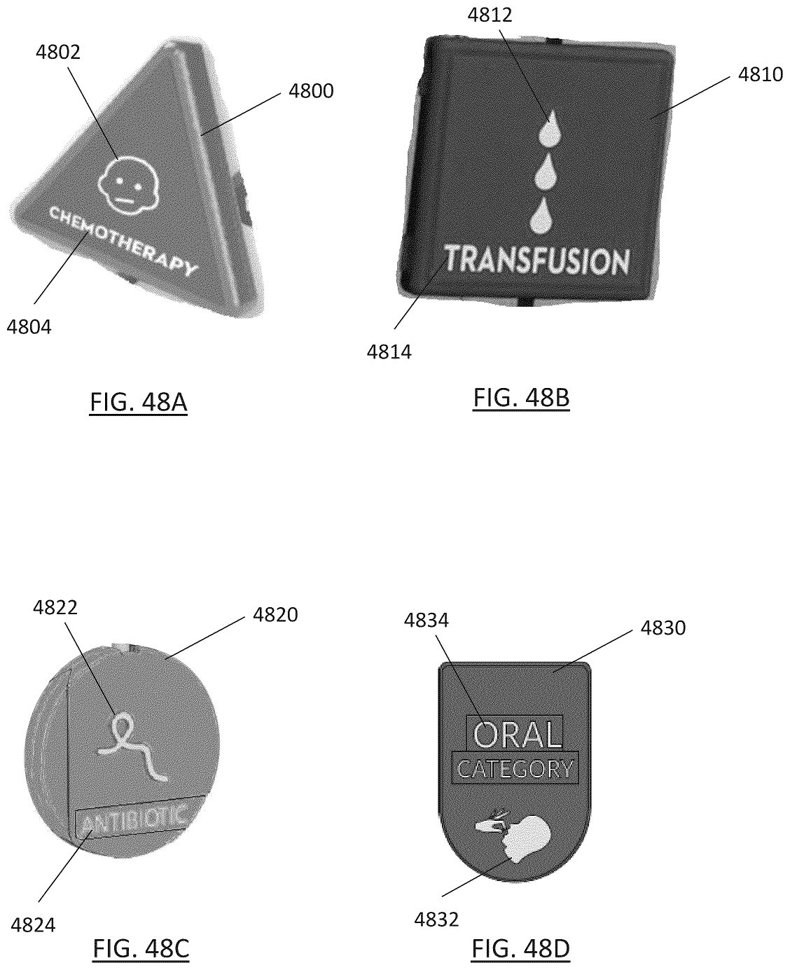

FIG. 48A shows an exemplary high alert indicator tag 4800 (e.g., having a first group of indicia including a color and shape similar to those of the tag 4700 described above with reference to FIG. 47A) having a second group of indicia including a haptic signature 4802 and a text label 4804 indicating that the tag 4800 is attached to an infusion therapy line carrying a chemotherapy medication. In other embodiments, a high alert indicator tag may include a second group of indicia that are indicative of other types of contents of an infusion therapy line such as opioids, antihypertensives, antiarrhythmics, anticoagulants, heparin, vasopressors, paralytics, insulin, sedatives, chemotherapy, potassium, and neuromuscular blockers.

FIG. 48B shows an exemplary specialty indicator tag 4810 (e.g., having a first group of indicia including a color and shape similar to those of the tag 4710 described above with reference to FIG. 47B) having a second group of indicia including a haptic signature 4812 and a text label 4814 indicating that the tag 4810 is attached to an infusion therapy line carrying a transfusion. In other embodiments, a specialty indicator tag may include a second group of indicia that are indicative of other types of contents or characteristics of an infusion therapy line such as obstetric care, pediatric dosing, blood product transfusions (e.g., red blood cells, platelets, plasma factors, whole blood), intravenous nutrition (e.g., total parenteral nutrition), or spinal administration.

FIG. 48C shows an exemplary non-high-alert indicator tag 4820 (e.g., having a first group of indicia including a color and shape similar to those of the tag 4720 described above with reference to FIG. 47C) having a second group of indicia including a haptic signature 4822 and a text label 4824 indicating that the tag 4820 is attached to an infusion therapy line carrying an antibiotic medication. In other embodiments, a non-high-alert alert indicator tag may include a second group of indicia that are indicative of other types of contents of an infusion therapy line such as antiemetics or other medications that do not fall into the high-alert category.

FIG. 48D shows an exemplary oral medication indicator tag 4830 (e.g., having a first group of indicia including a color and a shape similar to those of the tag of 4730 described above with reference to FIG. 47D) having a second group of indicia including a haptic signature 4832 and a text label 4834 indicating that the patient is receiving medication by oral administration. In some embodiments, rather than being configured to be attached to an infusion therapy tube, an oral medication indicator tag is configured to be positioned in another visible location near a patient, such as on an infusion therapy pole (e.g., at or near the top of an infusion therapy pole near the location where bags of infusion therapy fluid hang) or at a visible location on a patient's bed.

In some embodiments, an oral medication indicator tag configured to be indicative of (e.g., includes a haptic indicator and/or a text label indicative of) an oral biologic agents having antineoplastic and anticoagulant activity. Such medications are, in some situations, provided in lieu of infusion medications (e.g., intravenous infusion medications) in oncology, cardiology, and other fields, as they may be more precisely tailored to a specific malignancy (e.g., acute and chronic leukemia, solid tumor, etc.) and may therefore be more effective in reducing the growth of a malignancy than infusion therapy. However, the administration of such medications is not apparent to a caregiver unless the caregiver reads a patient's chart; there is no infusion therapy line to alert a caregiver as to the administration, let alone the nature, of such medications. It is desirable for a caregiver to be aware that a patient has been administered such medications in order to avoid possible drug interactions even with routine medications. Thus, in some embodiments, an oral medication indicator tag alerts a caregiver to the fact that a patient has been administered such a medication.

In some embodiments, in addition to the first and second groups of indicia described above, an exemplary indicator tag also includes a writable portion. FIGS. 49A, 49B, and 49C show a front view, a rear view, and an opened view showing both the front and rear, respectively, of an exemplary tag 4900 including a writable portion 4910 as well as a first group of indicia (e.g., as formed by the overall shape and color of the tag 4900 as described above with reference to FIGS. 47A-C) and a second group of indicia 4920, shown attached to an infusion therapy line L. In some embodiments, the second group of indicia 4920 is positioned on a first side of the exemplary indicator tag 4900 (e.g., a “front” side”) and the writable portion 4910 is positioned on an opposing second side of the exemplary indicator tag 4900 (e.g., a “rear” side”). In some embodiments, the writable portion 4910 is formed by material of the indicator tag 4900. In some embodiments, the writable portion 4910 is formed by a further material that is fixed to the tag (e.g., adhered by an adhesive, overmolded, pad-printed, decorated, press-fit, etc.). In some embodiments, such as shown in FIG. 49B, the writable portion 4910 has a color that differs from a color of the tag 4900. In such embodiments, the color difference provides a visual cue to the user indicating the purpose of the writable portion 4910. In some embodiments, the writable portion 4910 is a white portion fixed to an indicator tag 4900 that is not white (e.g., is red, blue, yellow, orange, green, or purple, as described above). In some embodiments, the writable portion 4910 may be used to carry information concerning the contents of the infusion therapy line to which the tag 4900 is affixed that is at a higher degree of detail or specificity than the first and second groups of indicia described above. For example, in some embodiments, the second group of indicia 4920 as shown in FIG. 49A may indicate that an infusion therapy line carries an opioid, and the writable portion 4910 may be used to identify the specific opioid carried by the infusion therapy line (e.g., fentanyl, morphine, methadone, etc.).

Thus, in some embodiments, an exemplary indicator tag provides a user with different levels of information concerning the contents of the infusion therapy line to which the indicator tag is attached. For example, referring to the exemplary tag 4900 shown in FIGS. 49A and 49B, the general shape and color of the indicator tag 4900 (e.g., the first group of indicia described above) allows a user to ascertain, from only a brief glance and/or from a distance (e.g., from across a room), and potentially also in low-visibility situations, that the contents of a infusion therapy line to which the indicator tag 4900 attached are a high-alert medicament. Thus, in some embodiments, immediate cognitive awareness of the presence and shape of the different type of tags attached to infusion therapy lines will instantly inform a caregiver or other individual of the significance of the therapy being administered. This is regardless of the specific medication being infused (e.g., this provides a first tier of information, bird's eye view or general awareness, of the scope of the care). In some embodiments, such awareness may give pause to the caregiver as to the level of caution needed in interacting with the patient (for example, upon seeing a triangular shaped “high alert” tag) of the possibility of potential serious drug interactions and potential risks associated with any procedure that is planned.

Continuing to refer to the exemplary tag 4900 shown in FIGS. 49A and 49B, the second group of indicia 4920 (e.g., a metaphoric haptic indicator and text label) allow a user to ascertain, with further inspection, that the contents of the infusion therapy line are an opioid medication. In some embodiments, the uniqueness of the differently shaped tags will lead the caregiver to inspect the tag and to read any additional information written upon the tag (e.g., to obtain a second tier of information). In some embodiments, such a second tier of information will be provided by the tags by virtue of their unique haptic signatures, colors, legible text, recognizable images, an/or luminescent qualities, as described above. In some embodiments, this will allow a caregiver to become more cognitively aware of the infusion line he/she needs to use and the potential contents of the line. In some embodiments, this information will also provide further insight as to the possibility of potential serious drug interactions and potential risks associated with any procedure that is planned (as when an anticoagulant tag is hanging on the line). Because of the minimal effort involved, this second tier of information is easily acquired and should, inevitably, lead to more informed care and, thereby, to better and safer patient care.

Continuing to refer to the exemplary tag 4900 shown in FIGS. 49A and 49B, the writable portion 4910 allows a user to ascertain, upon still closer inspection, that the infusion therapy line to which the tag 4900 is attached carries fentanyl. In some embodiments, this written material represents a “third tier” of information that provides a clinician with further insight as to the granular details of specific medications that are being administered to a patient.

Additionally, in some embodiments, the exemplary tags described herein (e.g., the exemplary tags 4810, 4820, 4830, and 4840 shown in FIGS. 48A-D) provide a “fourth tier” of information to clinicians who see a given patient on a regular basis. For example, in some embodiments, depending on the clinical progress of a given patient, additional tags of various types may either be added to the infusion therapy lines for the given patient (indicating worsening health) or may be removed from the infusion therapy lines for the given patient (indicating improving health).

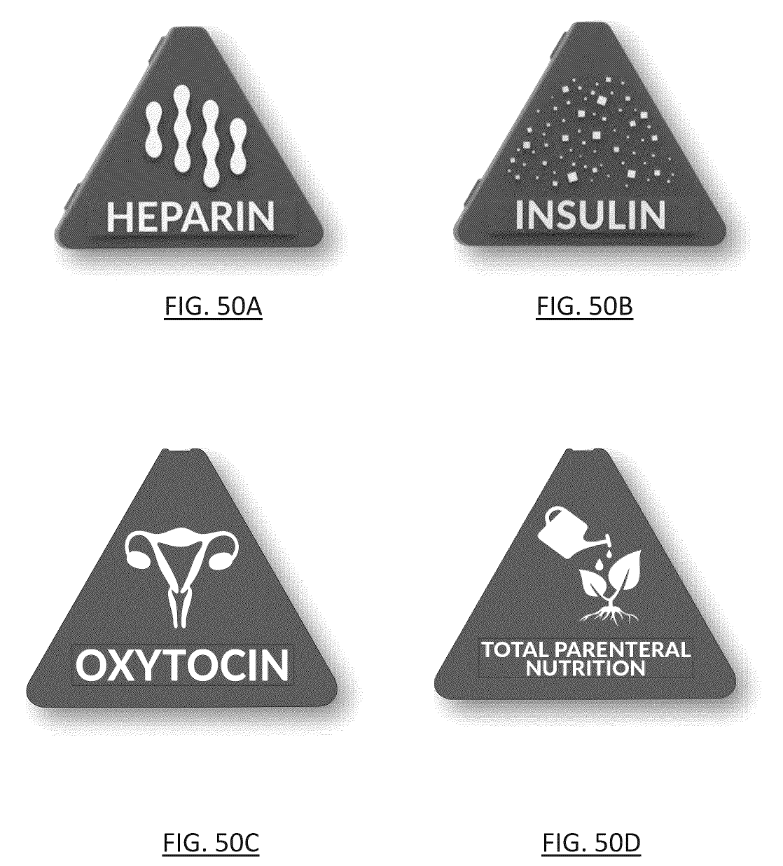

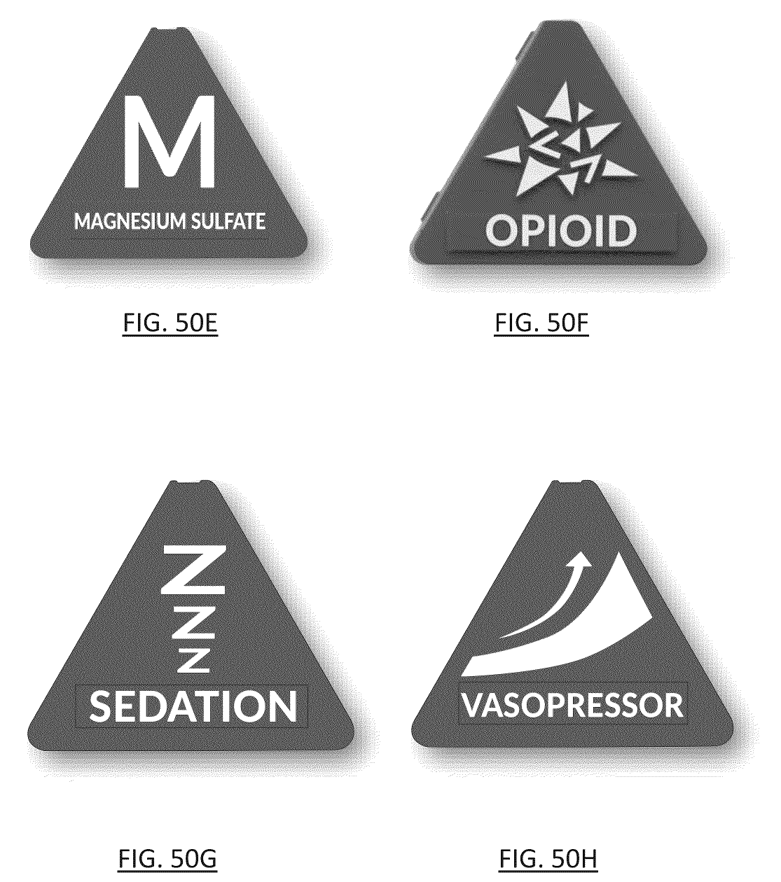

FIGS. 50A-50I show exemplary embodiments of tags indicating infusion lines that are utilized to deliver high-alert therapy. As used herein, “high-alert” refers to the categorization provided by the Institute for Safe Medication Practices List of High Alert Medications In Acute Care Settings. The tags shown in FIGS. 50A-50I include a specific color (e.g., red, yellow, or blue) and a specific shape (e.g., triangle) that are indicative of high-alert therapy.

Continuing to refer to FIGS. 50A-50I, FIG. 50A shows a tag indicative of heparin. FIG. 50B shows a tag indicative of insulin. FIG. 50C shows a tag indicative of oxytocin. FIG. 50D shows a tag indicative of total parenteral nutrition. FIG. 50E shows a tag indicative of magnesium sulfate. FIG. 50F shows a tag indicative of an opioid. FIG. 50G shows a tag indicative of a sedative. FIG. 50H shows a tag indicative of a vasopressor. FIG. 50I shows a tag indicative of chemotherapy. In the embodiment shown in FIG. 50I, the tag indicative of chemotherapy has a different color (e.g., yellow) than the color (e.g., red) used for tags indicative of other types of high-alert therapy as discussed above.

FIGS. 51A and 51B show tags indicating infusion lines that are utilized to deliver specialty therapy. The tags shown in FIGS. 51A and 51B include a specific color (e.g., blue) and a specific shape (e.g., square) that are indicative of specialty therapy. As used herein, “specialty” refers to categories of therapy including contents or characteristics of an infusion therapy line such as obstetric care, pediatric dosing, blood product transfusions (e.g., red blood cells, platelets, plasma factors, whole blood), intravenous nutrition (e.g., total parenteral nutrition), or spinal administration. FIG. 51A shows a tag indicating a transfusion. FIG. 51B shows a tag indicating lipids.

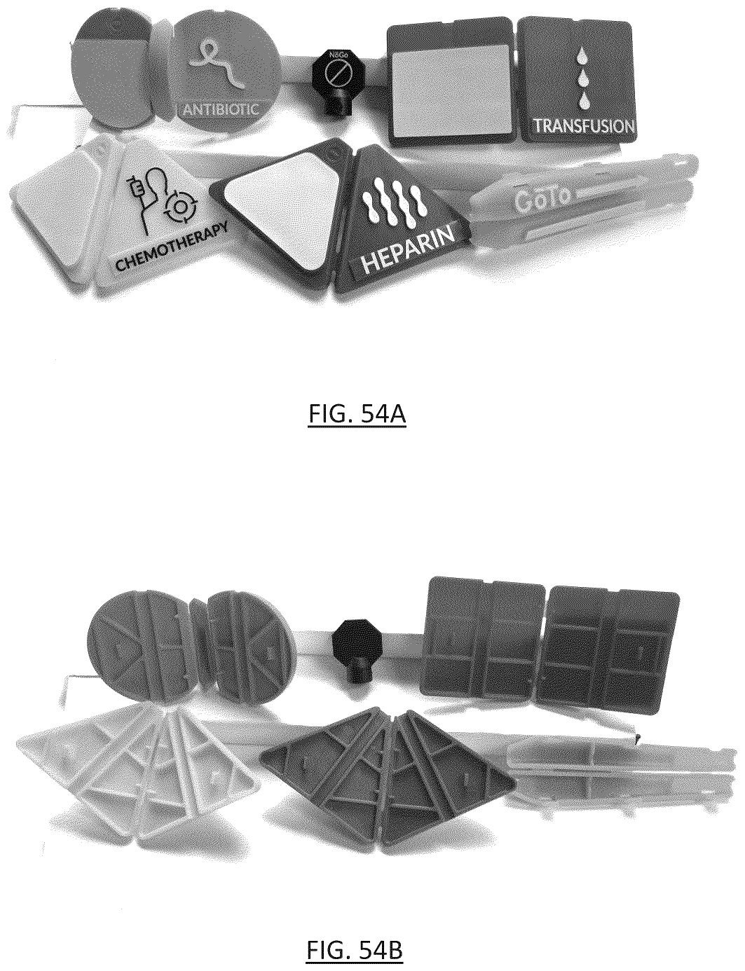

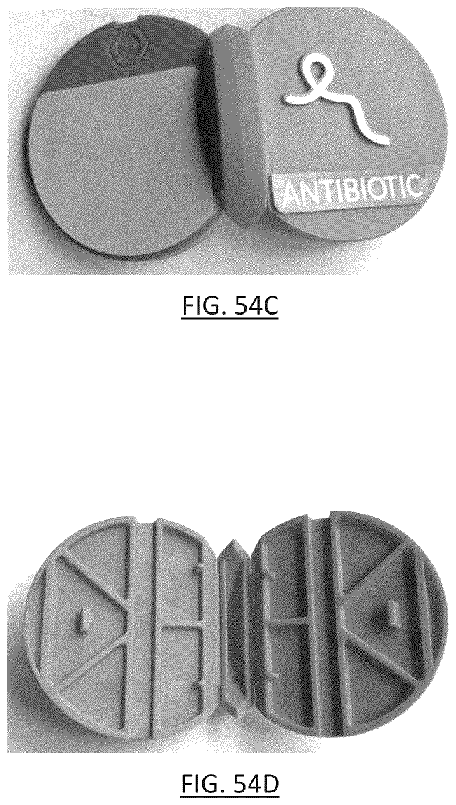

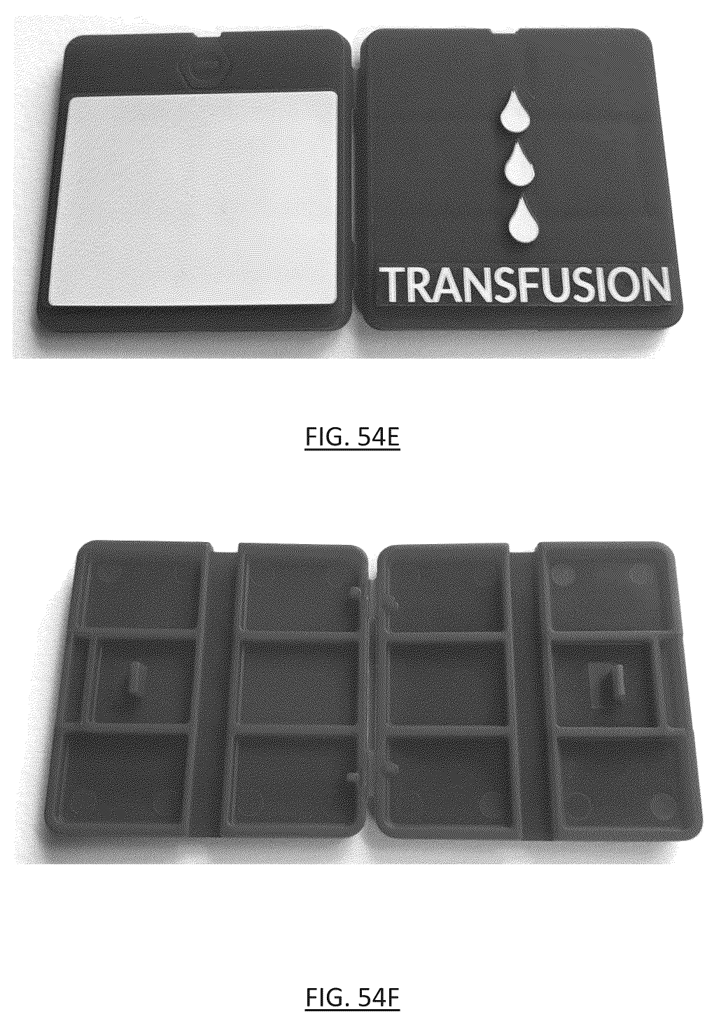

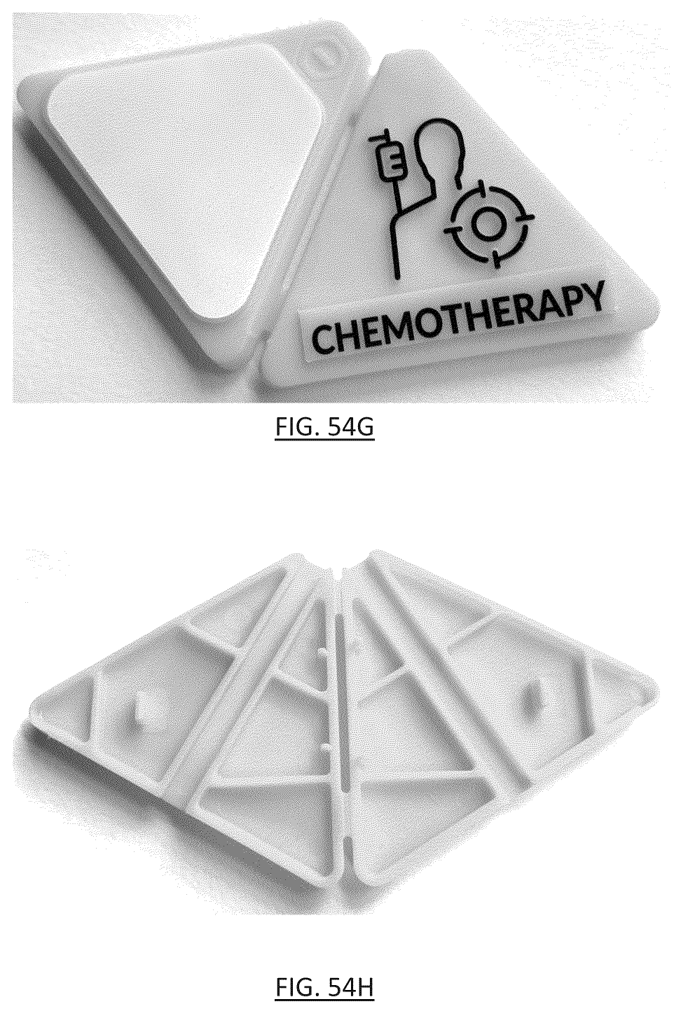

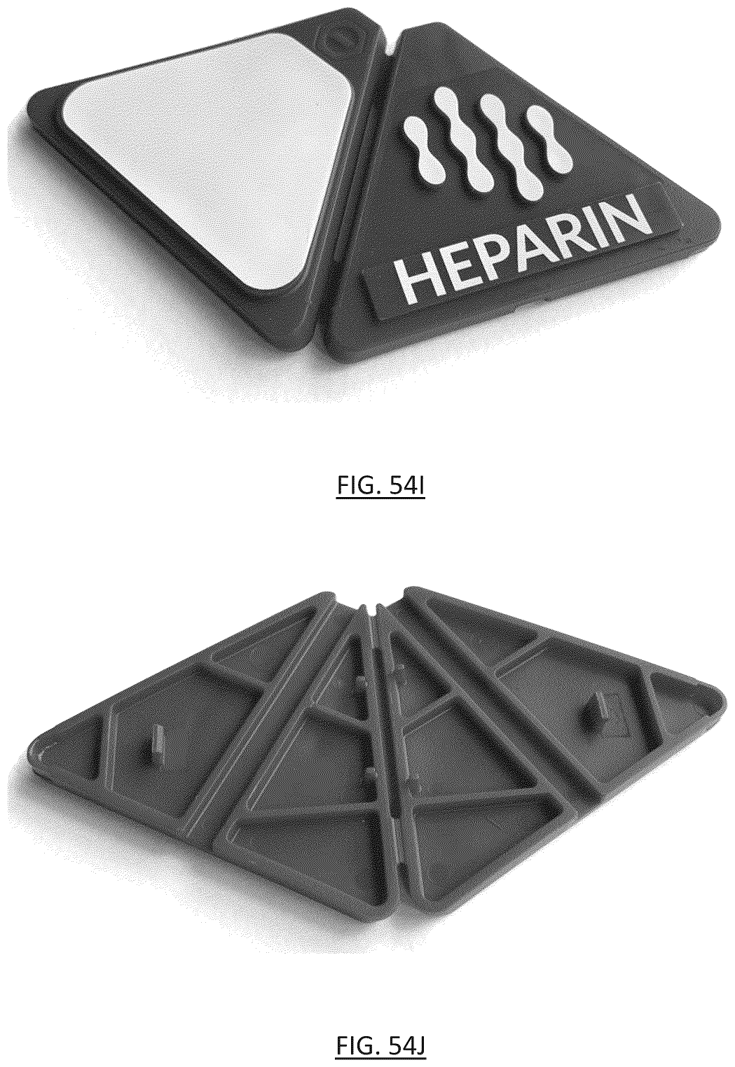

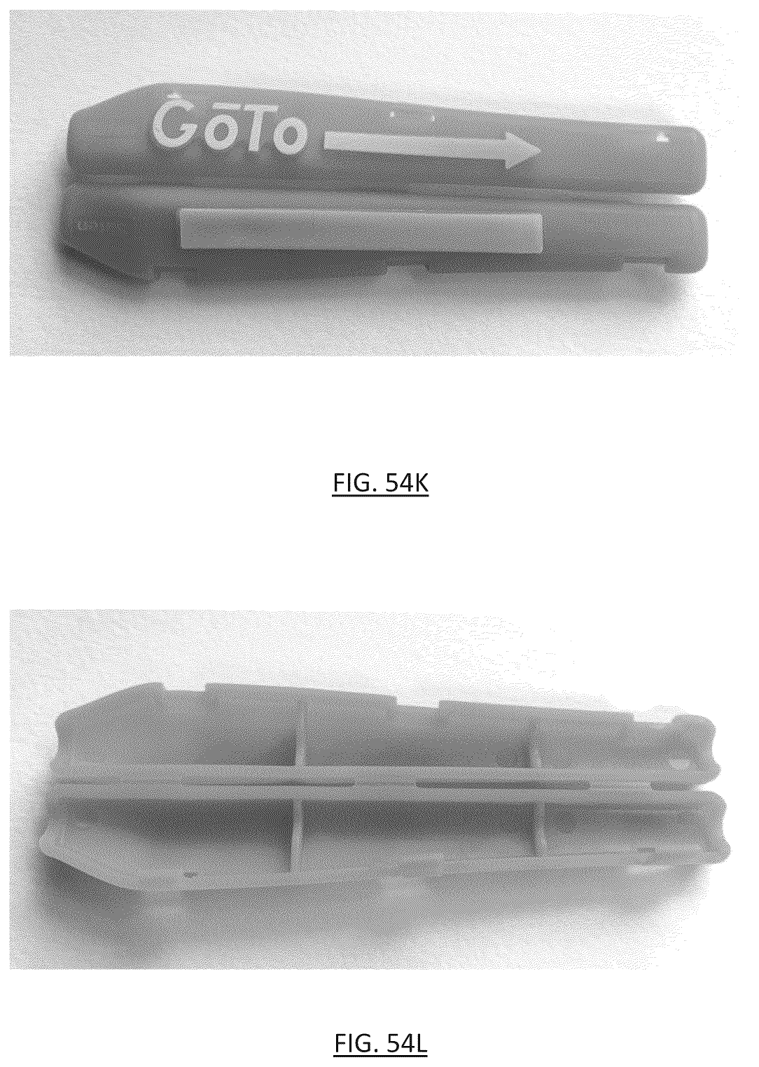

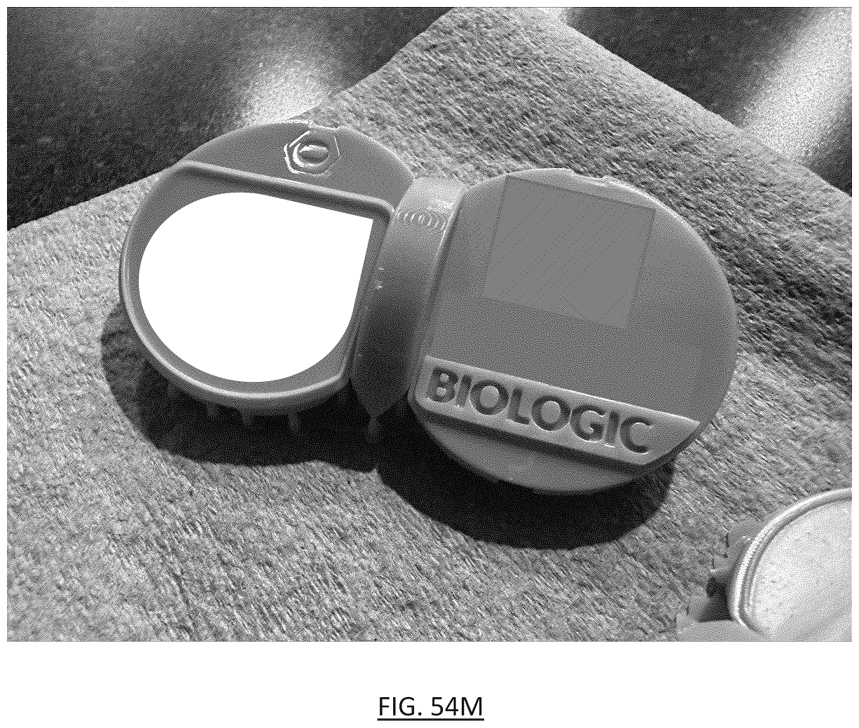

FIGS. 52A, and 52B show tags indicating infusion lines that are utilized to deliver non-high-alert therapy. The tags shown in FIGS. 52A and 52B include a specific color (e.g., orange) and a specific shape (e.g., circle) that are indicative of non-high-alert therapy. As used herein, “non-high-alert” refers to classes of medications that are not considered “high alert” as defined above. FIG. 52A shows a tag indicating antibiotics. FIG. 52B shows a tag indicating biologics. In some embodiments, such as discussed hereinafter with reference to FIG. 54M, a tag indicating biologics is colored dark green rather than orange.

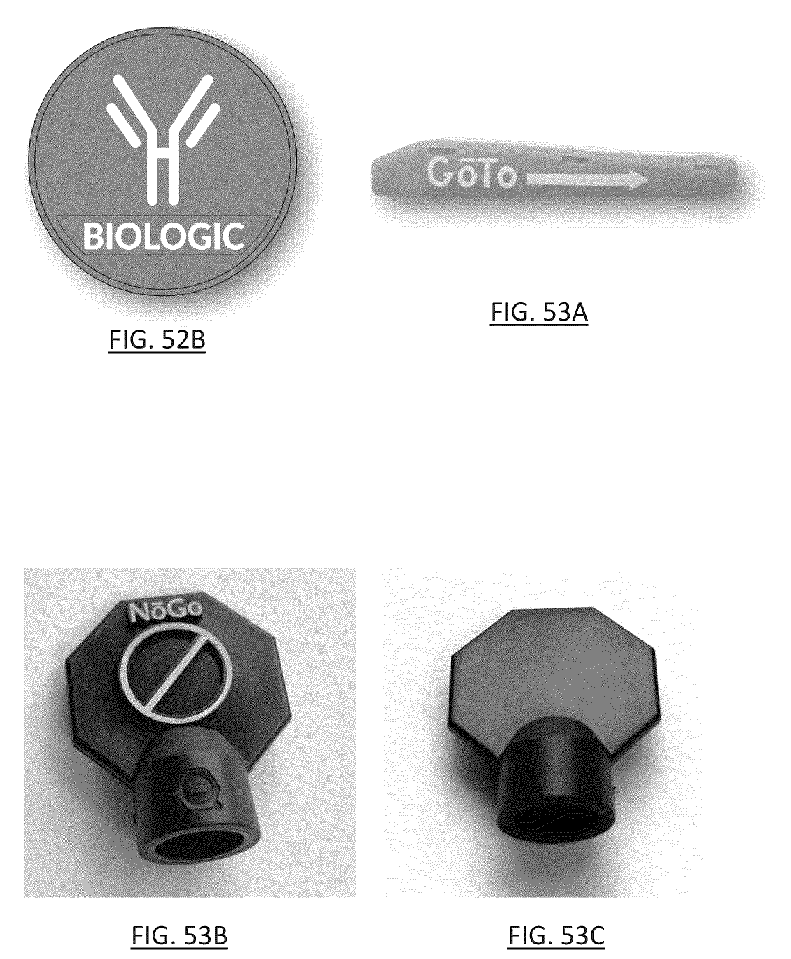

FIGS. 53A-53C show tags indicating specific line applications. FIG. 53A shows a tag indicating an infusion line that is to be used for the administration of emergency medication. The tag shown in FIG. 53A includes a specific color (e.g., bright green) and a specific shape (e.g., an arrow shape) that are indicative of administration of emergency medication. FIGS. 53B and 53C a front view and a rear view, respectively, of a port cover tag configured to cover a port and indicating that the port is not to be used for needleless therapy administration. The tag shown in FIGS. 53B and 53C includes a specific color (e.g., black) and a specific shape (e.g., an octagonal “stop sign” shape) that are indicative of a port that is not to be used.

In some embodiments, various tags as described herein are provided as part of a set including multiple types of tags having different meanings from one another. FIGS. 54A and 54B show an exemplary tag set including various tags taken from among those shown in FIGS. 50A-53C as described above. In some embodiments, tags include front and rear portions that can be separated from one another (e.g., opened) and joined back together (e.g., closed) so as to affix to an infusion therapy line. FIG. 54A shows the fronts of exemplary tags when opened as described above. FIG. 54B shows the backs of exemplary tags when opened as described above. FIGS. 54C and 54D show an opened front view and an opened rear view, respectively, of a prototype non-high-alert tag indicative of an antibiotic such as that described above with reference to FIG. 52A. FIGS. 54E and 54F show an opened front view and an opened rear view, respectively, of a prototype specialty tag indicative of a transfusion such as that described above with reference to FIG. 51A. FIGS. 54G and 54H show an opened front view and an opened rear view, respectively, of a prototype high-alert tag indicative of chemotherapy such as that described above with reference to FIG. 50I. FIGS. 54I and 54J show an opened front view and an opened rear view, respectively, of a prototype high-alert tag indicative of heparin such as that described above with reference to FIG. 50A. FIGS. 54K and 54L show an opened front view and an opened rear view, respectively, of a prototype specialty tag indicative of an emergency infusion line such as that described above with reference to FIG. 53A. FIG. 54M shows an opened front view of a prototype non-high-alert tag indicative of a biologic such as that described with reference to FIG. 3D. In the embodiment shown in FIG. 54M, the color green is indicative of a non-high-alert therapy (e.g., a biologic).

In one embodiment, the locations of the tags are: (1) near the attachment spike or connector to the bag of fluid; (2) near the peristaltic pump insert or the flow rate adjusting roller clamp, and (3) near the access port at the distal end of tube, closest to the needle/catheter connection to the patient.

In one embodiment, the locations of the tags also indicate the direction of fluid flow. In one embodiment, the tags include an embossed arrow indicating the direction of fluid flow.

In another embodiment, the tags are attached to the line such that: (1) the tags will not move along the plastic tube; (2) the tags will not interfere with the use of the line to deliver fluids at specified rate and to keep the vein open; (3) the tags will not interfere with any other function involving patient care, and (4) the tags are visible.

In some embodiments, indicator tags as described herein are made so as to be able to be secured to different sizes of infusion therapy tubes without sliding along the tubes and without restricting fluid flow along the tubes. In some embodiments, indicator tags as described herein include an internal channel that is sized and shaped to accommodate infusion therapy tubes therein. In some embodiments, indicator tags as described herein include a channel that is sized and shaped to accommodate infusion tags having outside diameters in a range of from 0.145 inches (e.g., a common small adult-size infusion therapy tube for adult dosing) to 0.165 inches (e.g., a common large adult-size infusion therapy tube for adult dosing).

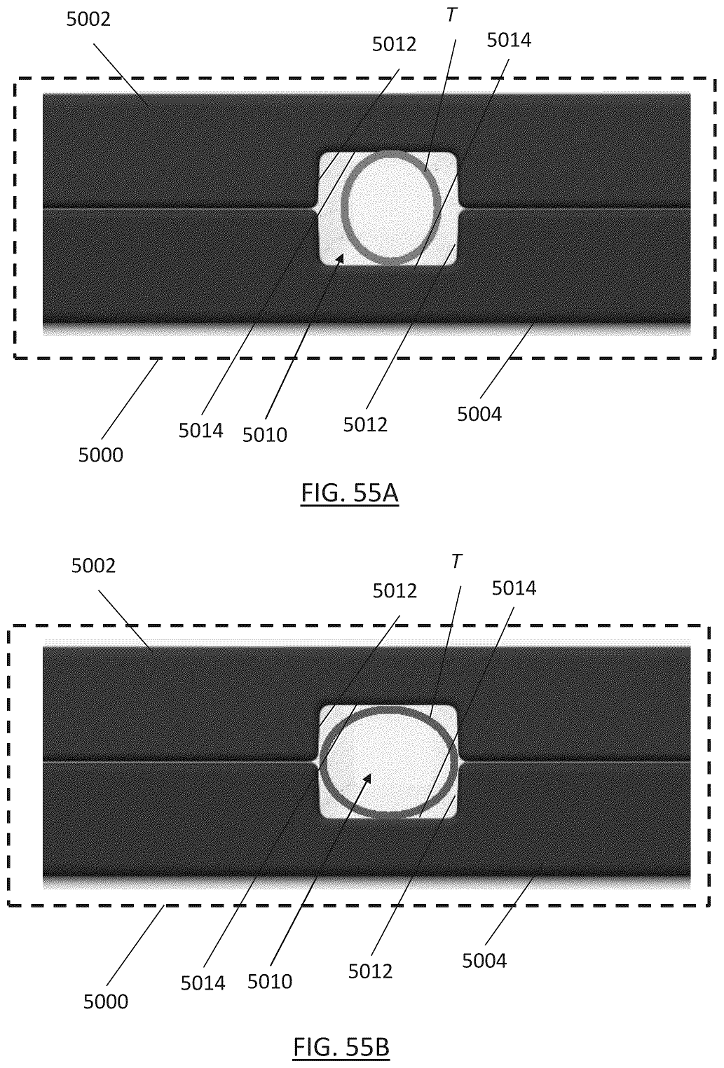

FIG. 55A shows a cross-sectional view of an indicator tag 5000. In some embodiments, the indicator tag 5000 includes a first portion 5002 and a second portion 5004 that form the indicator tag 5000. In some embodiments, the first portion 5002 and the second portion 5004 are joined to one another by a hinge (e.g., a living hinge). In some embodiments, a channel 5010 is formed by the joining of the first portion 5002 and the second portion 5004 to form the indicator tag 5000. In some embodiments, the channel 5010 defines a path that is configured to receive an infusion therapy tube. In some embodiments, the channel 5010 has a generally rectangular cross-section defined by shorter sides 5012 and longer sides 5014 as shown in FIG. 55A. In some embodiments, the channel 5010 has a rectangular cross-section. In some embodiments, the channel 5010 has a rectangular cross-section with rounded corners.

In some embodiments, the channel 5010 has a generally rectangular cross-section, and the dimensions of the generally rectangular cross-section are sized so as to cause the indicator tag 5000 to clamp to an infusion therapy tube positioned within the channel 5010 in a manner such that the indicator tag 5000 remains in place along the infusion therapy tube and does not constrain fluid flow along the infusion therapy tube. In some embodiments, the shorter sides 5012 of the generally rectangular cross-section have a side length that is about equal to a smallest outside diameter infusion therapy tube to which the indicator tag 5000 is configured to attach. For example, in embodiments in which the indicator tag 5000 is configured to attach to an infusion therapy tube having a smallest outside diameter of 0.145 inches, the shorter sides 5012 have a side length that is in a range of from 0.135 inches to 0.145 inches. In such embodiments, such as shown in FIG. 55A, the longer sides 5014 firmly hold to an infusion therapy tube T and do not substantially compress or otherwise deform the infusion therapy tube T.

In some embodiments, the longer sides 5014 of the generally rectangular cross-section of the channel 5010 have a side length that is about 10% greater than a largest outside diameter infusion therapy tube to which the indicator tag 5000 is configured to attach. For example, in embodiments in which the indicator tag 5000 is configured to attach to an infusion therapy tube having a largest outside diameter of 0.165 inches, the longer sides 5014 have a side length that is about 0.18 inches, e.g., between 0.175 inches and 0.185 inches. In such embodiments, such as shown in FIG. 55B, an infusion therapy tube T is compressed between the longer sides 5014, and flexes outward toward the shorter sides 5012 in the space provided by the longer sides 5014 being longer than the outside diameter of the infusion therapy tube T.

In some embodiments in which the indicator tag 5000 is configured to attach to an infusion therapy tube having a smallest outside diameter of 0.145 inches, the channel 5010 has shorter sides 5012 having a length that is in a range of from 0.1 inches to 0.145 inches, or is in a range of from 0.11 inches to 0.145 inches, or is in a range of from 0.12 inches to 0.145 inches, or is in a range of from 0.125 inches to 0.145 inches, or is in a range of from 0.13 inches to 0.145 inches, or is in a range of from 0.135 inches to 0.145 inches, or is in a range of from 0.14 inches to 0.145 inches, or is in a range of from 0.14 inches to 0.15 inches, or is in a range of from 0.144 inches to 0.146 inches, or is about 0.145 inches, or is 0.145 inches.

In some embodiments in which the indicator tag 5000 is configured to attach to an infusion therapy tube having a smallest outside diameter of X, the channel 5010 has shorter sides 5012 having a length that is in a range of from 0.9*X to X, or is in a range of from 0.92*X to X, or is in a range of from 0.94*X to X, or is in a range of from 0.96*X to X, or is in a range of from 0.97*X to X, or is in a range of from 0.98*X to X, or is in a range of from 0.99*X to X, or is in a range of from 0.98*X to 1.02*X, or is in a range of from 0.99*X to 1.01*X, or is about equal to X, or is equal to X.

In some embodiments in which the indicator tag 5000 is configured to attach to an infusion therapy tube having a largest outside diameter of 0.165 inches, the channel 5010 has longer sides 5014 having a length that is in a range of from 0.17 inches to 0.19 inches, or is in a range of from 0.175 inches to 0.185 inches, or is in a range of from 0.16 inches to 0.18 inches, or is in a range of from 0.165 inches to 0.18 inches, or is in a range of from 0.17 inches to 0.18 inches, or is in a range of from 0.175 inches to 0.18 inches, or is in a range of from 0.18 inches to 0.2 inches, or is in a range of from 0.18 inches to 0.195 inches, or is in a range of from 0.18 inches to 0.19 inches, or is in a range of from 0.18 inches to 0.185 inches, or is equal to about 0.18 inches, or is equal to 0.18 inches.

In some embodiments in which the indicator tag 5000 is configured to attach to an infusion therapy tube having a largest outside diameter of Y, the channel 5010 has longer sides 5014 having a length that is in a range of from 1.05*Y to 1.3*Y, or is in a range of from 1.1*Y to 1.3*Y, or is in a range of from 1.15*Y to 1.3*Y, or is in a range of from 1.2*Y to 1.3*Y, or is in a range of from 1.25*Y to 1.3*Y, or is in a range of from 1.05*Y to 1.25*Y, or is in a range of from 1.1*Y to 1.25*Y, or is in a range of from 1.15*Y to 1.25*Y, or is in a range of from 1.2*Y to 1.25*Y, or is in a range of from 1.05*Y to 1.2*Y, or is in a range of from 1.1*Y to 1.2*Y, or is in a range of from 1.15*Y to 1.2*Y, or is in a range of from 1.05*Y to 1.15*Y, or is in a range of from 1.1*Y to 1.15*Y, or is in a range of from 1.05*Y to 1.1*Y, or is about equal to 1.09*Y, or is equal to 1.09*Y, or is about equal to 1.1*Y, or is equal to 1.1*Y.

In some embodiments in which the indicator tag 5000 is configured to attach to an infusion therapy tube having a smallest outside diameter of X and a largest outside diameter of Y, the channel 5010 has longer sides 5014 having a length that is in a range of from 1.05*Y to 1.3*Y and has shorter sides 5012 having a length that is in a range of from 0.9*X to X, or has longer sides 5014 having a length that is in a range of from 1.05*Y to 1.15*Y and has shorter sides 5012 having a length that is in a range of from 0.9*X to X, or has longer sides 5014 having a length that is in a range of from 1.05*Y to 1.3*Y and has shorter sides 5012 having a length that is in a range of from 0.95*X to 1.05*X.

In some embodiments, each of the tags provides multisensory inputs (e.g., tactile inputs and visual inputs), including multiple types of visual inputs (e.g., color, legible text, recognizable image, etc.), to a clinician. In some embodiments, such multisensory inputs enable enhanced and quickened recognition of a tag, and corresponding enhanced and quickened recognition of the content of a given infusion therapy line, as compared to prior techniques such as labels written on medical tape. For example, in some embodiments, a tag including a tactile input provides enhanced recognition in low-light situations or situations where a label is positioned in a location that is difficult to view.

In another embodiment, exemplary tags are packaged in a kit including at least two of the exemplary tags. In some embodiments, a kit includes at least one tag corresponding to each of at least two different medicaments (e.g., at least one of the indicator 300A shown in FIGS. 24A and 24B, at least one of the indicator 300B shown in FIGS. 25A and 25B, at least one of the indicator 300C shown in FIGS. 26A and 26B, etc.). In some embodiments, a kit includes at least one GoTo Line tag such as the indicator 200 shown in FIGS. 14-23. In some embodiments, a kit includes at least one port cover tag such as the indicator 300F shown in FIG. 29. In some embodiments a kit includes tags as described above, along with instructions for installation and use of such tags in accordance with the exemplary embodiments described herein. In some embodiments, a kit includes at least one tag corresponding to each of a set of different medicaments that are the most common medicaments delivered via infusion therapy (e.g., intravenously). In some embodiments, a kit includes three tags for each of at least two different medicaments, wherein the three tags for each specific medicament are provided for use (A) near the attachment spike or connector to the bag of fluid containing the specific medicament; (B) near the peristaltic pump insert or the flow rate adjusting roller clamp, and (C) near the access port at the distal end of tube, closest to the needle/catheter connection to the patient. In some embodiments, a kit includes instructions for use of the at least two exemplary tags included in such a kit.

In some embodiments, a kit includes at least a first tag including a haptic signature that includes an embossed image and a second tag including a haptic signature that includes a debossed image. In some embodiments, a kit includes at least a first tag including a haptic signature that includes an embossed image and a second tag including a haptic signature that includes a textured portion. In some embodiments, a kit includes at least a first tag including a haptic signature that includes a textured portion and a second tag including a haptic signature that includes a debossed image. In some embodiments, a kit includes at least a first tag including a haptic signature that includes an embossed image and a second tag including a haptic signature that includes a debossed portion within an embossed image. In some embodiments, a kit includes at least a first tag including a haptic signature that includes an embossed image and a second tag including a haptic signature that includes an embossed portion within a debossed image. In some embodiments, a kit includes at least a first tag including a haptic signature that includes a debossed image and a second tag including a haptic signature that includes a debossed portion within an embossed image. In some embodiments, a kit includes at least a first tag including a haptic signature that includes a debossed image and a second tag including a haptic signature that includes an embossed portion within a debossed image. In some embodiments, a kit includes at least a first tag including a haptic signature that includes a textured portion and a second tag including a haptic signature that includes a debossed portion within an embossed image. In some embodiments, a kit includes at least a first tag including a haptic signature that includes a textured portion and a second tag including a haptic signature that includes an embossed portion within a debossed image. In some embodiments, a kit includes at least a first tag including a haptic signature that includes an embossed image, a second tag including a haptic signature that includes a debossed image, and a third tag including a haptic signature that includes a textured portion.

For the GoTo Line to be safe, it must only carry an acceptable fluid, such as normal saline, lactated Ringers, etc., which will not adversely affect administration of emergency medication. For the GoTo Line to be effective, the flow must be sufficient to keep the vein open. In an embodiment, permanent, clear, unambiguous markings of the GoTo Line keeps the GoTo Line readily recognizable and thereby suitable and safe for subsequent use; and enables confident and timely location of the GoTo Line when it is required in an emergency.

The proper function of the entire system relies on situation-specific design characteristics. In one embodiment, these include (1) high visibility; (2) distinctive shapes; (3) colors which assist in locating the line against drapes, equipment, bedding, etc.; (4) Low light visibility (luminescence); (5) text that provides an additional corroboration of the medicament in the infusion therapy line; (6) texture that has association with the contents of the medicament in the infusion therapy line; (7) location of tags on the infusion therapy line set (A) near the attachment spike or connector to the bag of fluid; (B) near the peristaltic pump insert or the flow rate adjusting roller clamp, and (C) near the access port at the distal end of tube, closest to the needle/catheter connection to the patient. In such an embodiment, a system of haptic tags is suitable for blind operation under high-stress use scenarios, with augments such as color and labeling providing secondary cues to provide safety redundancy.

Being able to act expeditiously with accuracy and confidence is helpful to the medical professional both in crisis situations, as well as during the performance of routine daily tasks. In a crisis, quick and accurate action to administer proper medications to the right location can save a patient's life. During the performance of daily routine tasks, a multi-sensory system that communicates a plurality of confirmatory messages to the care-giver's brain increases the likelihood that mistakes will be detected and avoided that could otherwise result from the lull of repetitive tasks under seemingly “normal” or routine circumstances. Multi-sensory input tends to compensate for variations in the degree of reliance upon any particular sense among care-giving personnel. For example, color may elicit a stronger recognition response in one person than another. If only color is used as a differentiator, a person not particularly attuned to color may not perform as well over the course of repetitive activities. However, when color, tag shape, patterns, and textures associated with a particular medication are simultaneously deployed, that same individual may be much more likely to avoid an error even during the dullest or most tiresome circumstances, especially when other tags provide contrast and contradiction as yet another confirmation-by-contrast sensory input.

Also, it is believed that receiving a multitude of confirmatory and distinguishing signals allows the brain to quickly achieve a sense of confidence in identification. This alleviates the need to laboriously investigate, ponder and assess; having achieved a sense of confidence from multiple-sensory inputs frees up “bandwidth” to proceed with other tasks without confusion and undue delay. Thus, routine tasks can be accomplished with speed, efficiency and accuracy, all while increasing the likelihood of “catching” and avoiding potential errors due to fatigue or monotony.

The exemplary embodiments comprise a multi-sensory feedback system to allow medical professionals to expeditiously assess, distinguish and confirm the current status of multiple medication inputs for a patient. Thus, the combination of lines and tags can communicate a holistic picture of current medication status, while the inputs from an individual line provide quick certainty as to its content.

By deploying the exemplary system throughout a care-giving environment and/or user group, such as a hospital network, military or veteran medical service, or nationally if standardized, a uniform system is provided to train staff to act expeditiously with greater confidence and accuracy in the administration of infusion therapy medicaments. As staff become familiar with the colors, tag shapes, textures, patterns, and text used to indicate particular families of medications, their speed of recognition and ability to act correctly, promptly, and with confidence increases. Thus, the multi-sensory feedback system provides an improved method of managing medication administration safely and accurately.

The particular colors, shapes, patterns and/or textures deployed as infusion therapy tags can be selected from a wide range of possibilities. Those disclosed in the figures and described herein are merely illustrative of the possibilities. Once selected, it is preferred that such tags be uniformly deployed in a given facility to ensure consistency and maximize the speed and accuracy of recognition. The tags placed along any individual infusion therapy tube ideally employ a particular color, texture, pattern or haptic signature and text that differs from and contrasts with the same characteristics of the tags placed along other infusion therapy lines in use. Both the affirmative communication achieved by the tags on an individual line, as well as the contrasts communicated from neighboring lines, add redundancy to the sensory communications that inform the healthcare worker of the content and status of the lines. By deploying such a system, and using it as a tool to instruct attending healthcare professionals, one can achieve an augmented degree of safety and reliability compared to traditional labeling systems.

### Example

Study Description: To evaluate the effectiveness of the exemplary embodiments in improving time to administration and accuracy of emergent bolus drug administration, a pilot study was designed using a simulated scenario replicating the conditions that typically exist in a medical transport or ICU setting (low lighting, motion, cramped quarters, complex multiple infusion therapy access sites, life sustaining infusion regimens).

Methods: A prospective, randomized crossover study was performed in the simulation lab at the Centre for Emergency Health Sciences in Spring Branch, Texas. Study participants received a 4-minute video orientation on a tag system in accordance with the exemplary embodiments prior to beginning the study. The following scenario was then presented: The “patient” (a mannequin instrumented with a triple lumen central venous catheter, four peripheral IV catheters, a double channel IV pump, an endotracheal tube, a chest tube, a nasogastric tube to suction, and a Foley catheter) requires a STAT dose of IV midazolam to treat status epilepticus. The appropriate line to administer midazolam contains normal saline and is labeled using (in the case of the control) a standard, tape labeled IV-line or (in the case of the experiment) a tag in accordance with the exemplary embodiments. A photograph of the experimental tag in accordance with the exemplary embodiments is shown as FIG. 44. The experimental tag in accordance with the exemplary embodiments included a haptic signature that is embossed at an embossing height of 0.015 inch. The time to administration was measured by two independent observers from the time the participant entered the simulator to when the midazolam syringe content was pushed into the infusion line.

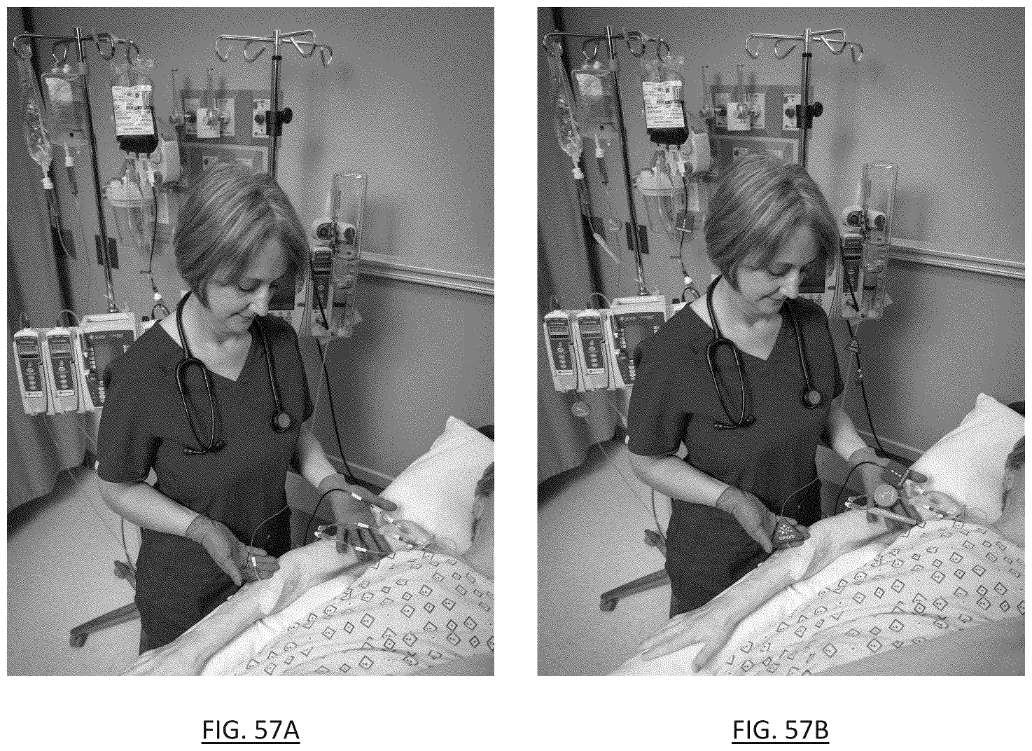

Each participant served as their own control by performing the simulation twice, in random order: once with the tape labeled infusion therapy line and once with tag in accordance with the exemplary embodiments. FIG. 57A shows a representative image of a clinician working with infusion lines that have been labeled with tape. FIG. 57B shows a representative image of a clinician working with infusion therapy lines that have been labeled in accordance with exemplary embodiments described herein. The simulator environment was appropriately noisy, with low lighting and a confined work space, as may be experienced in an ICU or during medical transport. To minimize learning, the appropriate infusion line was rotated systematically between the four IV sites, and ten (10) minutes separated the two simulations. Each encounter was videotaped, and an exit questionnaire captured participants' subjective impressions of the tag in accordance with the exemplary embodiments.

Subjects were dichotomized by arm and descriptive statistics were used to compare demographics between groups. Student T-tests were used to compare mean time to medication delivery, and differences between nurses, paramedics and physicians. Order bias (i.e., based on whether the tag in accordance with the exemplary embodiments was first or second in sequence) was also assessed using T-tests.

Results: For all subjects combined, the mean (+/−SD) time to administration of midazolam labeled with a tag in accordance with the exemplary embodiments (22.79±14.04 seconds) was significantly faster than the mean (+/−SD) time to administration of midazolam labeled with tape as a control (37.08±14.49 seconds; p=0.0001). Among paramedics and nurses, administration time was significantly faster statistically, whereas for physicians there was a faster administration (as indicated in Table 1 below with p>0.05). The difference was not attributable to whether the subject received the tag in accordance with the exemplary embodiments during the first or second simulation. One participant chose the incorrect line during their control/tape labeled simulation (i.e., a medication error). In the exit survey, the participants overwhelmingly concluded that the tag in accordance with the exemplary embodiments improved confidence and ease in identifying the appropriate line and that its use will improve patient safety.

TABLE 1

| | | | | | | Exemplary | Exemplary |
|---|---|---|---|---|---|---|---|
| | GoTo | | | | | Tag faster # | Tag |
| Practitioner | Tag | | Control | | P | practitioners | % faster |
| (n) | (sec) | | (sec) | | Value | (%) | (sec) |
| MD (4) | 26.8 | (11.9) | 35.5 | (16.5) | 0.17 | 3 (75%) | 14.6% |
| Medic (10) | 23.9 | (14.3) | 38.4 | (10.6) | <0.05 | 9 (90%) | 37.3% |
| Nurse (10) | 20.1 | (15.4) | 36.4 | (18.1) | <0.05 | 9 (90%) | 44.7% |
| Total (24) | 22.8 | (14.0) | 37.1 | (14.5) | <0.05 | 21 (88%) | 36.6% |

Conclusion: The pilot study results suggest the tag in accordance with the exemplary embodiments allows STAT drug administration to occur on average 36.6% faster than the tape labeled line.

As will be understood by those familiar with the art, the invention may be embodied in other specific forms without departing from the spirit or essential characteristics thereof. Likewise, the particular naming and division of the modules, features, attributes, methodologies, managers and other aspects are not mandatory or significant, and the mechanisms that implement the invention or its features may have different names, divisions and/or formats.

It should be emphasized that the above-described embodiments of the present disclosure are merely possible examples of implementations that are set forth for a clear understanding of the principles of the disclosure. Many variations and modifications may be made to the above-described embodiment(s) without departing substantially from the spirit and principles of the disclosure. It can be appreciated that several of the above-disclosed and other features and functions, or alternatives thereof, may be desirably combined into many other different systems or applications. All such modifications and variations are intended to be included herein within the scope of this disclosure.

What is claimed is:

1. A system of medical indicators, comprising:
a set of indicator tags,
wherein each of the indicator tags comprises:
a body having a first end, a second end opposite the first end, a first surface extending from the first end to the second end, and an aperture extending from the first end to the second end,
wherein the aperture is sized and shaped to receive an infusion tube,
a first plurality of identifying characteristics that are indicative of a category of therapy,
a second plurality of identifying characteristics that are indicative of a specific therapy within the category of therapy,
wherein the set of indicator tags comprises:
a first indicator tag comprising:
the first plurality of identifying characteristics that are indicative of a first category of therapy, and
the second plurality of identifying characteristics that are indicative of a first specific therapy within the first category of therapy,
a second indicator tag comprising:
the first plurality of identifying characteristics that are indicative of the first category of therapy, and
the second plurality of identifying characteristics that are indicative of a second specific therapy within the first category of therapy,
a third indicator tag comprising:
the first plurality of identifying characteristics that are indicative of a second category of therapy, and
the second plurality of identifying characteristics that are indicative of a first specific therapy within the second category of therapy, and
a fourth indicator tag comprising:
the first plurality of identifying characteristics that are indicative of the second category of therapy, and
the second plurality of identifying characteristics that are indicative of a second specific therapy within the second category of therapy.

2. The system of claim 1, wherein the first plurality of identifying characteristics comprises an indicator tag shape and an indicator tag color.

3. The system of claim 1, wherein the second plurality of identifying characteristics comprises an icon and a text label.

4. The system of claim 3, wherein the icon is a haptic icon.

5. The system of claim 3,
wherein each of the indicator tags further comprises a second surface opposite the first surface,
wherein the icon and the text label are positioned on the first surface, and
wherein each of the indicator tags further comprises a writable portion on the second surface.

6. The system of claim 1, wherein the first category of therapy is a first one of high-alert therapy, non-high-alert therapy, or specialty therapy, and wherein the second category of therapy is a second one of high-alert therapy, non-high-alert therapy, or specialty therapy.

7. The system of claim 6, wherein the first category of therapy is high-alert therapy, and wherein the first plurality of identifying characteristics that are indicative of the first category of therapy comprise a triangular tag shape and a red tag color.

8. The system of claim 7, wherein one of:
(a) the second category of therapy is non-high-alert therapy and the first plurality of identifying characteristics that are indicative of the second category of therapy comprise a circular tag shape and an orange tag color, or
(b) the second category of therapy is specialty therapy and the first plurality of identifying characteristics that are indicative of the second category of therapy comprise a square tag shape and a blue tag color.

9. The system of claim 7, wherein the first specific therapy within the first category of therapy is one of heparin, insulin, an opioid, oxytocin, total parenteral nutrition, magnesium sulfate, a sedative, or chemotherapy.

10. The system of claim 1, further comprising:
a fifth indicator tag comprising identifying characteristics that are indicative of an emergency line.

11. A system of medical indicators, comprising:
a set of indicator tags,
wherein each of the indicator tags comprises:
a body having a first end, a second end opposite the first end, a first surface extending from the first end to the second end, and an aperture extending from the first end to the second end,
wherein the aperture is sized and shaped to receive an infusion tube,
wherein the aperture is configured such that each of the indicator tags can be secured to at least a first size of the infusion tube and a second size of the infusion tube without sliding along the infusion tube or restricting fluid flow within the infusion tube,
wherein the first size of the infusion tube has a first outside diameter,
wherein the second size of the infusion tube has a second outside diameter, and
wherein the second outside diameter is greater than the first outside diameter, and
a plurality of identifying characteristics that are indicative of a therapy.

12. The system of claim 11, wherein the first size of the infusion tube is a small adult-size infusion tube, and wherein the second size of the infusion tube is a large adult-size infusion tube.

13. The system of claim 11, wherein the aperture has a generally rectangular cross-section comprising shorter sides and longer sides.

14. The system of claim 13, wherein the aperture has a rectangular cross-section with rounded corners.

15. The system of claim 13, wherein the shorter sides have a shorter side length that is about equal to the first outside diameter.

16. The system of claim 15, wherein the shorter side length is in a range of from 0.9 times the first outside diameter to 1 times the first outside diameter.

17. The system of claim 13, wherein the longer sides have a longer side length that is in a range of from 1.05 times the second outside diameter to 1.3 times the second outside diameter.

18. The system of claim 17, wherein the shorter sides have a shorter side length that is in a range of from 0.9 times the first outside diameter to 1 times the first outside diameter.

19. The system of claim 13, wherein the first outside diameter is 0.145 inches and the second outside diameter is 0.165 inches.

20. The system of claim 13,
wherein the shorter sides have a shorter side length that is in a range of from 0.14 inches to 0.15 inches, and
wherein the longer sides have a longer side length that is in a range of from 0.175 inches to 0.185 inches.
